SEQUENCE LISTING (12 sequences)

SEQ ID NO: 1 - protein, 9 aa, Artificial Sequence
  MC-38-derived neoantigen - Adpgk
ASMTNMELM

SEQ ID NO: 2 - protein, 9 aa, Artificial Sequence
  MC-38-derived neoantigen - Reps1
AQLANDVVL

SEQ ID NO: 3 - protein, 9 aa, Artificial Sequence
  MC-38-derived neoantigen - Irgq
AALLNSAVL

SEQ ID NO: 4 - protein, 9 aa, Artificial Sequence
  MC-38-derived neoantigen - Cpne1
SSPYSLHYL

SEQ ID NO: 5 - protein, 9 aa, Artificial Sequence
  MC-38-derived neoantigen - Aaltf
MAPIDHTTM

SEQ ID NO: 6 - protein, 9 aa, Artificial Sequence
  B16.F10-derived neoantigen - Obsl1
LCPGNKYEM

SEQ ID NO: 7 - protein, 10 aa, Artificial Sequence
  B16.F10-derived neoantigen - Snx5
AAFQKNLIEM

SEQ ID NO: 8 - protein, 9 aa, Artificial Sequence
  B16.F10-derived neoantigen - Pbk
AAVILRDAL

SEQ ID NO: 9 - protein, 8 aa, Artificial Sequence
  B16.F10-derived neoantigen - Atp11a
QSLGFTYL

SEQ ID NO: 10 - protein, 15 aa, Artificial Sequence
  B16.F10-derived neoantigen - Eef2
VKAYLPVNESFAFTA

SEQ ID NO: 11 - protein, 8 aa, Artificial Sequence
  Maraba N52-59 peptide
RGYVYQGL

SEQ ID NO: 12 - protein, 9 aa, Artificial Sequence
  FMT N301-309 peptide
AVVLMFAQC

(19) United States
(12) Patent Application Publication
(10) Pub. No.: US 2022/0160800 A1
(43) Pub. Date: May 26, 2022

(54) METHODS FOR INDUCING AN IMMUNE RESPONSE AGAINST NEOANTIGENS

(71) Applicants: TURNSTONE BIOLOGICS INC., Ottawa (CA); CHILDREN'S HOSPITAL OF EASTERN ONTARIO RESEARCH INSTITUTE INC., Ottawa (CA)

(72) Inventors: David Stojdl, Ottawa (CA); Justyna KMIECIK, Ottawa (CA); Michael F. BURGESS, Chester, NJ (US)

(73) Assignees: TURNSTONE BIOLOGICS INC., Ottawa, ON (CA); CHILDREN'S HOSPITAL OF EASTERN ONTARIO RESEARCH INSTITUTE INC., Ottawa, ON (CA)

(21) Appl. No.: 17/440,671

(22) PCT Filed: Mar. 19, 2020
(86) PCT No.: PCT/CA2020/050366

§ 371 Date: Sep. 17, 2021

Related U.S. Application Data
(60) Provisional application No. 62/821,397, filed on Mar. 20, 2019.
(60) Provisional application No. 62/892,532, filed on Aug. 27, 2019.

(51) Int. Cl. A61K 35/761; A61K 35/766; A61K 35/768; A61P 35/00; A61P 37/04

(52) U.S. Cl. CPC A61K 35/761; A61K 35/766; C07K 7/06; A61P 35/00; A61P 37/04; A61K 35/768

(57) ABSTRACT

In one aspect, provided herein is a heterologous boost method for inducing an immune response to at least one neoantigen, the method comprising administering to a subject a first boost and subsequently administering to the subject a second boost, wherein the first boost comprises a first oncolytic virus comprising a genome that expresses, in the subject, a first peptide, or the first boost comprises a first oncolytic virus and a second peptide, wherein the second boost comprises a second oncolytic virus comprising a genome that expresses, in the subject, a third peptide, or the second boost comprises a second oncolytic virus and a fourth peptide, wherein the first peptide, the second peptide, the third peptide, and the fourth peptide are each capable of inducing an immune response to at least one neoantigen, and wherein the second oncolytic virus is immunologically distinct from the first oncolytic virus. The subject may have pre-existing immunity to the at least one neoantigen. The subject may have been administered a priming composition before receiving the first boost, wherein the priming composition is capable of inducing an immune response to the at least one neoantigen.

This application claims the benefit of U.S. Provisional Application No. 62/821,397, filed Mar. 20, 2019 and U.S. Provisional Application No. 62/892,532, filed Aug. 27, 2019 each of which is incorporated by reference herein in its entirety.

This application incorporates by reference a Sequence Listing submitted with this application as a text file in ASCII format entitled “14596-002-228_ST25.txt” created on Mar. 19, 2020 and having a size of 2,906 bytes.

In one aspect, provided herein is a heterologous boost method for inducing an immune response to at least one neoantigen, the method comprising administering to a subject a first boost and subsequently administering to the subject a second boost, wherein the first boost comprises a first oncolytic virus comprising a genome that expresses, in the subject, a first peptide, or the first boost comprises a first oncolytic virus and a second peptide, wherein the second boost comprises a second oncolytic virus comprising a genome that expresses, in the subject, a third peptide, or the second boost comprises a second oncolytic virus and a fourth peptide, wherein the first peptide, the second peptide, the third peptide, and the fourth peptide are each capable of inducing an immune response to at least one neoantigen, and wherein the second oncolytic virus is immunologically distinct from the first oncolytic virus. The subject may have pre-existing immunity to the at least one neoantigen. The subject may have been administered a priming composition before receiving the first boost, wherein the priming composition is capable of inducing an immune response to the at least one neoantigen.

### BACKGROUND

An oncolytic prime:boost strategy based on a single tumour antigen target can achieve robust protection in the prophylactic setting. Yet in the therapeutic setting, tumour-bearing animal model systems demonstrate rapid tumour regression following oncolytic immunotherapy but often fail to achieve long-term cures, with tumours recurring following treatment. Several additional in vivo studies have shown similar outcomes following immunotherapeutic approaches based on a single antigen target. This effect can be the result of antigen loss in response to therapeutic pressure (Rommelfanger et al., Cancer Res. 2012; 72(18):4753-4764; Khong et al., J Immunother. 2004; 27(3):184-190; Mackensen et al., J Clin Oncol. 2006; 24(31):5060-5069; Yee C et al., Proc Natl Acad Sci USA. 2002; 99(25):16168-16173). However, antigen-targeted T cell therapies can still fail to generate durable cures in 80-90% of animals even when tumours continue to robustly express the targeted antigen, and relapsed tumours can regain responsiveness to antigen-targeted therapies following tumour re-transplantation into naive animals (Straetemans et al., Mol Ther. 2015; 23(2):396-406), suggesting a role for immunosuppressive mechanisms in addition to bona fide antigen loss. Since immunotherapies targeted towards more than one tumour antigen typically achieve longer-term control (Rommelfanger et al., Cancer Res. 2012; 72(18):4753-4764; Anurathapan et al., Mol Ther. 2014; 22(3):623-633; Hegde et al., Mol Ther. 2013; 21(11):2087-2101), there is clear therapeutic value in exploring large-scale tumour antigen library targets.

Neoepitopes are peptide epitopes that arise from the genetic aberrations within the tumour. These mutations convert self epitopes that would otherwise be tolerated by T cells in the periphery into immunogenic foreign epitopes capable of engaging circulating T cells. Importantly, this means that neoantigen-specific CD8+ T cells often show exquisite specificity for mutant (non-self) over wild-type (self) proteins (Nielsen et al., Clin Cancer Res. 2016; 22(9):2226-2236).

Tumours are genetically complex tissues that present with extreme levels of inter- and intra-patient heterogeneity. Multiple clones ranging from 2 to >20 (depending on the cancer indication) can be identified within a single tumour (Andor et al., Nat Med. 2016; 22(1):105-113; Ling et al., Proc Natl Acad Sci USA. 2015; 112(47):E6496-6505). Multi-sample whole exome sequencing analysis demonstrates that a single tumour mass has an extremely high genetic diversity, with more than 1,000,000 mutations in coding regions (Ling et al., Proc Natl Acad Sci USA. 2015; 112 (47):E6496-6505). Between 8-78% of neoantigens are located in specific subclonal populations (McGranahan N, Furness AJ, Rosenthal R, et al. Clonal neoantigens elicit T cell immunoreactivity and sensitivity to immune checkpoint blockade. Science. 2016; 351(6280):1463-1469).

In human patients, increased neoantigen load is associated with elevated frequencies of CD8+ T cells at the tumour site (Brown et al., Genome research. 2014; 24(5):743-750), and tumour neoantigen burden correlates with overall survival following checkpoint blockade (McGranahan N, Furness A J, Rosenthal R, et al. Clonal neoantigens elicit T cell immunoreactivity and sensitivity to immune checkpoint blockade. Science. 2016; 351(6280):1463-1469; Brown et al., Genome research. 2014; 24(5):743-750; Strickland et al., Oncotarget. 2016; 7(12):13587-13598; Rizvi et al., Science. 2015; 348(6230):124-128; Giannakis et al., Genomic Correlates of Immune-Cell Infiltrates in Colorectal Carcinoma. Cell Rep. 2016; 15(4):857-865). Thus, there is clear therapeutic value in targeting neoantigens in the oncolytic vaccine setting.

### SUMMARY

In one aspect, provided herein is a method of inducing an immune response to at least one neoantigen in a subject, the method comprising: (a) administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen; and (b) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the second oncolytic virus is immunologically distinct from the first oncolytic virus, and wherein the third and fourth compositions are administered concurrently or sequentially to the subject. In a specific embodiment, the subject has pre-existing immunity to the at least one neoantigen. In another embodiment, the subject has previously been administered a dose of a priming composition that is capable of inducing an immune response to the at least one neoantigen. In one embodiment, the step (b) is performed 7 to 21 days after step (a). In another embodiment, step (b) is performed 2 weeks to 3 months after step (a). In some embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In some embodiments, the third oncolytic virus and the fourth oncolytic virus are immunologically distinct from the first oncolytic virus.

In certain embodiments, the first protein or fragment thereof is capable of inducing an immune response to two or more different neoantigens. In some embodiments, the first protein comprises at least one epitope of each of the two or more neoantigens. In certain embodiments, the first protein encoded by the first transgene includes at least one proteasomal cleavage site. In some embodiments, the first protein encoded by the first transgene is a fusion protein.

In certain embodiments, the first peptide composition is capable of inducing an immune response to two or more different neoantigens. In a specific embodiment, the first peptide composition comprises two peptides, wherein one of the peptides comprises at least one epitope of one of the neoantigens, and the other peptide comprises at least one epitope of the other neoantigen.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In certain embodiments, the amino acid sequence of the second peptide composition is different from the amino acid sequence of first protein, the first peptide composition, or both. In other embodiments, the amino acid sequence of the second peptide composition is identical to the amino acid sequence of first protein, the first peptide composition, or both. In some embodiments, the amino acid sequence of the second peptide composition includes at least one epitope found in the first peptide composition.

In another aspect, provided herein is a method of inducing an immune response to at least one neoantigen in a subject with pre-existing immunity to the at least one neoantigen, or a subject who has previously been administered a dose of a priming composition that is capable of inducing an immune response to the at least one neoantigen, the method comprising: (a) administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and (b) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus. In a specific embodiment, the subject has pre-existing immunity to the at least one neoantigen. In another embodiment, the subject has previously been administered a dose of a priming composition that is capable of inducing an immune response to the at least one neoantigen. In one embodiment, the step (b) is performed 7 to 21 days after step (a). In another embodiment, step (b) is performed 2 weeks to 3 months after step (a). In some embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In some embodiments, the third oncolytic virus and the fourth oncolytic virus are immunologically distinct from the first oncolytic virus.

In certain embodiments, the first protein or fragment thereof is capable of inducing an immune response to two or more different neoantigens. In some embodiments, the first protein comprises at least one epitope of each of the two or more neoantigens. In certain embodiments, the first protein encoded by the first transgene includes at least one proteasomal cleavage site. In some embodiments, the first protein encoded by the first transgene is a fusion protein.

In certain embodiments, the first peptide composition is capable of inducing an immune response to two or more different neoantigens. In a specific embodiment, the first peptide composition comprises two peptides, wherein one of the peptides comprises at least one epitope of one of the neoantigens, and the other peptide comprises at least one epitope of the other neoantigen.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different than the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In certain embodiments, the amino acid sequence of the second peptide composition is different than the amino acid sequence of first protein, the first peptide composition, or both. In other embodiments, the amino acid sequence of the second peptide composition is identical to the amino acid sequence of first protein, the first peptide composition, or both. In some embodiments, the amino acid sequence of the second peptide composition includes at least one epitope found in the first peptide composition.

In another aspect, provided herein is a method of inducing an immune response to at least one neoantigen in a subject, comprising administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the third and fourth compositions are administered concurrently or sequentially to the subject, wherein the subject has pre-existing immunity to the at least one neoantigen, or the subject was previously administered a dose of a priming composition that is capable of inducing an immune response to the at least one neoantigen, and wherein the subject was previously administered a first boost comprising a dose of a first composition, wherein the first composition comprises a first oncolytic virus comprising a genome that comprises a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus. In one embodiment, the first boost was administered to the subject 7 to 21 days before the second boost. In another embodiment, the first boost was administered to the subject 2 weeks to 3 months before the second boost. In some embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In some embodiments, the third oncolytic virus and the fourth oncolytic virus are immunologically distinct from the first oncolytic virus.

In certain embodiments, the first protein or fragment thereof is capable of inducing an immune response to two or more different neoantigens. In some embodiments, the first protein comprises at least one epitope of each of the two or more neoantigens. In certain embodiments, the first protein encoded by the first transgene includes at least one proteasomal cleavage site. In some embodiments, the first protein encoded by the first transgene is a fusion protein.

In certain embodiments, the first peptide composition is capable of inducing an immune response to two or more different neoantigens. In a specific embodiment, the first peptide composition comprises two peptides, wherein one of the peptides comprises at least one epitope of one of the neoantigens, and the other peptide comprises at least one epitope of the other neoantigen.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In certain embodiments, the amino acid sequence of the second peptide composition is different from the amino acid sequence of first protein, the first peptide composition, or both. In other embodiments, the amino acid sequence of the second peptide composition is identical to the amino acid sequence of first protein, the first peptide composition, or both. In some embodiments, the amino acid sequence of the second peptide composition includes at least one epitope found in the first peptide composition.

In another aspect, provided herein is a method of inducing an immune response to at least one neoantigen in a subject, comprising administering to the subject a second boost comprising a dose of a fourth composition, wherein the fourth composition comprises a second oncolytic virus that comprises a genome comprising a first transgene, wherein the first transgene encodes and expresses a first protein that is expressed in the subject, wherein the first protein or a fragment thereof is capable of inducing an immune response to the at least one neoantigen, wherein the subject has pre-existing immunity to the at least one neoantigen, or the subject was previously administered a dose of a priming composition that is capable of inducing an immune response to the at least one neoantigen, and wherein the subject was previously administered a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject, wherein the first peptide composition is capable of inducing an immune response to the at least one neoantigen, and wherein the second oncolytic virus is immunologically distinct from the first oncolytic virus. In one embodiment, the first boost was administered to the subject 7 to 21 days before the second boost. In another embodiment, the first boost was administered to the subject 2 weeks to 3 months before the second boost. In some embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In some embodiments, the third oncolytic virus and the fourth oncolytic virus are immunologically distinct from the first oncolytic virus.

In certain embodiments, the first protein or fragment thereof is capable of inducing an immune response to two or more different neoantigens. In some embodiments, the first protein comprises at least one epitope of each of the two or more neoantigens. In certain embodiments, the first protein encoded by the first transgene includes at least one proteasomal cleavage site. In some embodiments, the first protein encoded by the first transgene is a fusion protein.

In certain embodiments, the first peptide composition is capable of inducing an immune response to two or more different neoantigens. In a specific embodiment, the first peptide composition comprises two peptides, wherein one of the peptides comprises at least one epitope of one of the neoantigens, and the other peptide comprises at least one epitope of the other neoantigen.

In another aspect, provided herein is a method of inducing an immune response to at least one neoantigen in a subject with pre-existing immunity to the neoantigen, or a subject who has previously been administered a dose of a priming composition that is capable of inducing an immune response to the at least one neoantigen, the method comprising: (a) administering to the subject a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject; and (b) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the fifth and sixth compositions are administered concurrently or sequentially to the subject, wherein the first and second peptide compositions are each capable of inducing an immune response to the at least one neoantigen, and wherein the second oncolytic virus is immunologically distinct than the first oncolytic virus.

In some embodiments, the first oncolytic virus comprises a genome that comprises a first transgene, wherein the first transgene encodes and expresses a first protein in the subject, and wherein the first protein or a fragment thereof is capable of inducing an immune response to the at least one neoantigen. In certain embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen. In some embodiments, the method further comprises administering to the subject a third boost comprising a dose of a seventh composition, wherein the seventh composition comprises a third oncolytic virus comprising a genome that comprises a third transgene, wherein the third transgene encodes and expresses a third protein in the subject, wherein the third protein or a fragment thereof is capable of inducing an immune response to the at least one neoantigen, and wherein the third oncolytic virus is immunologically distinct from the second oncolytic virus. In certain embodiments, the method further comprises administering to the subject a third boost comprising: (i) a dose of a seventh composition comprising a third oncolytic virus and a third peptide composition; or (ii) a dose of an eighth composition and a dose of a ninth composition, wherein the eighth composition comprises the third oncolytic virus, and the ninth composition comprises the third peptide composition, wherein the eighth and ninth compositions are concurrently or sequentially administered to the subject, wherein the third peptide composition is capable of inducing an immune response to the at least one neoantigen, and wherein the third oncolytic virus is immunologically distinct from the second oncolytic virus. In specific embodiments, the third oncolytic virus is immunologically distinct from the first oncolytic virus.

In some embodiments, the first peptide composition and the second peptide composition each comprise an identical peptide. In certain embodiments, the first peptide composition and the second peptide composition each comprise a peptide, wherein the peptide of the first peptide composition comprises an amino acid sequence that overlaps with an amino acid sequence of the peptide of the second peptide composition. In some embodiments, the amino acid sequence of the second peptide composition includes at least one epitope found in the first peptide composition.

In some embodiments, the first peptide composition comprises two peptides and the second peptide composition comprises two peptides, wherein the two peptides of the first and second peptide compositions are identical. In certain embodiments, the first peptide composition comprises two peptides and the second peptide composition comprises two peptides, wherein the two peptides of the first and second peptide compositions each comprise overlapping amino acid sequences.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In another aspect, provided herein is a method of inducing an immune response to at least one neoantigen in a subject, comprising administering the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the fifth and sixth compositions are administered concurrently or sequentially to the subject, wherein the subject has pre-existing immunity to the at least one neoantigen, or the subject was previously administered a dose of a priming composition that is capable of inducing an immune response to the at least one neoantigen, and wherein the subject was previously administered a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject, wherein the first peptide composition and the second peptide composition are each capable of inducing an immune response to the at least one neoantigen, and wherein the first oncolytic virus is immunologically distinct from the second oncolytic virus.

In some embodiments, the first oncolytic virus comprises a genome that comprises a first transgene, wherein the first transgene encodes and expresses a first protein in the subject, and wherein the first protein or a fragment thereof is capable of inducing an immune response to the at least one neoantigen. In certain embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen. In some embodiments, the method further comprises administering to the subject a third boost comprising a dose of a seventh composition, wherein the seventh composition comprises a third oncolytic virus comprising a genome that comprises a third transgene, wherein the third transgene encodes and expresses a third protein in the subject, wherein the third protein or a fragment thereof is capable of inducing an immune response to the at least one neoantigen, and wherein the third oncolytic virus is immunologically distinct from the second oncolytic virus. In certain embodiments, the method further comprises administering to the subject a third boost comprising: (i) a dose of a seventh composition comprising a third oncolytic virus and a third peptide composition; or (ii) a dose of an eighth composition and a dose of a ninth composition, wherein the eighth composition comprises the third oncolytic virus, and the ninth composition comprises the third peptide composition, wherein the eighth and ninth compositions are concurrently or sequentially administered to the subject, wherein the third peptide composition is capable of inducing an immune response to the at least one neoantigen, and wherein the third oncolytic virus is immunologically distinct from the second oncolytic virus. In specific embodiments, the third oncolytic virus is immunologically distinct from the first oncolytic virus.

In some embodiments, the first peptide composition and the second peptide composition each comprise an identical peptide. In certain embodiments, the first peptide composition and the second peptide composition each comprise a peptide, wherein the peptide of the first peptide composition comprises an amino acid sequence that overlaps with an amino acid sequence of the peptide of the second peptide composition. In some embodiments, the amino acid sequence of the second peptide composition includes at least one epitope found in the first peptide composition. In some embodiments, the first peptide composition comprises two peptides and the second peptide composition comprises two peptides, wherein the two peptides of the first and second peptide compositions are identical. In certain embodiments, the first peptide composition comprises two peptides and the second peptide composition comprises two peptides, wherein the two peptides of the first and second peptide compositions each comprise overlapping amino acid sequences.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In another aspect, provided herein is a method of inducing an immune response to at least one neoantigen in a subject, comprising: (a) administering to the subject a dose of a priming composition that is capable of inducing an immune response to the at least one neoantigen; (b) subsequently administering to the subject a first boost comprising (i) a dose of a first composition, wherein the first composition comprises a first oncolytic virus and a first peptide composition, or (ii) a dose of a second composition and a dose of third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and (c) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the second peptide composition is capable of inducing an immune response to the at least one neoantigen, wherein the fifth and sixth compositions are administered concurrently or sequentially to the subject, and wherein second oncolytic virus is immunologically distinct from the first oncolytic virus. In certain embodiments, the first oncolytic virus comprises a genome that comprises a first transgene, wherein the first transgene encodes and expresses a first protein in the subject, and wherein the first protein or a fragment thereof is capable of inducing an immune response to the at least one neoantigen. In some embodiments, the second oncolytic virus comprises a genome that comprises a second transgene, wherein the second transgene encodes and expresses a second protein in the subject, and wherein the second protein or a fragment thereof is capable of inducing an immune response to the at least one neoantigen.

In certain embodiments, the method further comprises administering to the subject a third boost comprising a dose of a seventh composition, wherein the seventh composition comprises a third oncolytic virus comprising a genome that comprises a third transgene, wherein the third transgene encodes and expresses a third protein in the subject, wherein the third protein or a fragment thereof is capable of inducing an immune response to the at least one neoantigen, and wherein the third oncolytic virus is immunologically distinct from the second oncolytic virus. In some embodiments, the method further comprises administering to the subject a third boost comprising: (i) a dose of a seventh composition comprising a third oncolytic virus and a third peptide composition; or (ii) a dose of an eighth composition and a dose of a ninth composition, wherein the eighth composition comprises the third oncolytic virus, and the ninth composition comprises the third peptide composition, wherein the eighth and ninth compositions are concurrently or sequentially administered to the subject, wherein the third peptide composition is capable of inducing an immune response to the at least one neoantigen, and wherein the third oncolytic virus is immunologically distinct from the second oncolytic virus. In a specific embodiment, the third oncolytic virus is immunologically distinct from the first oncolytic virus.

In certain embodiments, the first peptide composition and the second peptide composition each comprise an identical peptide. In some embodiments, the first peptide composition and the second peptide composition each comprise a peptide, wherein the peptide of the first peptide composition comprises an amino acid sequence that overlaps with an amino acid sequence of the peptide of the second peptide composition. In some embodiments, the amino acid sequence of the second peptide composition includes at least one epitope found in the first peptide composition.

In certain embodiments, the first peptide composition comprises two peptides and the second peptide composition comprises two peptides, wherein the two peptides of the first and second peptide compositions are identical. In some embodiments, the first peptide composition comprises two peptides and the second peptide composition comprises two peptides, wherein the two peptides of the first and second peptide compositions each comprise overlapping amino acid sequences.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In another aspect, provided herein is a method of inducing an immune response to at least one neoantigen in a subject with pre-existing immunity to the at least one neoantigen, or a subject who has previously been administered a dose of a priming composition that is capable of inducing an immune response to the at least one neoantigen, the method comprising: (a) administering to the subject a first boost comprising a dose of a first composition, wherein the first composition comprises a first oncolytic virus that comprises a genome comprising a first transgene, wherein the first transgene encodes and expresses a first protein in the subject, and wherein the first protein or a fragment thereof is capable of inducing an immune response to the at least one neoantigen; and (b) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

In some embodiments, the first protein or fragment thereof and the second protein or fragment thereof are each capable of inducing an immune response to two or more different neoantigens. In certain embodiments, the first protein comprises at least one epitope of each of the two or more neoantigens, and the second protein comprises at least one epitope of each of the two or more neoantigens. In some embodiments, the first protein encoded by the first transgene, the second protein encoded by the second transgene, or both include at least one proteasomal cleavage site. In certain embodiments, the first protein encoded by the first transgene, the second protein encoded by the second transgene, or both are a fusion protein.

In some embodiments, the method further comprises administering the subject a third boost comprising (i) a dose of third composition comprising a third oncolytic virus that comprises a genome comprising a third transgene wherein the third transgene encodes and expresses a third protein in the subject, wherein the third protein or a fragment thereof is capable of inducing an immune response to the at least one neoantigen, (ii) a dose of fourth composition comprising a fourth oncolytic virus and a first peptide composition, wherein the first peptide composition is capable of inducing an immune response to the at least one neoantigen, or (iii) a dose of a fifth composition and a dose of a sixth composition, wherein the fifth composition comprises the fourth oncolytic virus, and the sixth composition comprises the first peptide composition, and wherein the third oncolytic virus and the fourth oncolytic virus are immunologically distinct from the second oncolytic virus. In a specific embodiment, the third oncolytic virus and the fourth oncolytic virus are immunologically distinct from the first oncolytic virus.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In another aspect, provided herein is a method of inducing an immune response to at least one neoantigen in a subject, the method comprising to the subject a second boost comprising a dose of a second composition, wherein the second composition comprises a second oncolytic virus that comprises a genome comprising a second transgene, wherein the second transgene encodes and expresses a second protein in the subject, wherein the second peptide or a fragment thereof is capable of inducing an immune response to the at least one neoantigen, wherein the subject has pre-existing immunity to the at least one neoantigen, or the subject was previously administered a dose of a priming composition that is capable of inducing an immune response to the at least one neoantigen, and wherein the subject was previously administered a first boost comprising a dose of a first composition, wherein the first composition comprises a first oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

In some embodiments, the first protein or fragment thereof and the second protein or fragment thereof are each capable of inducing an immune response to two or more different neoantigens. In certain embodiments, the first protein comprises at least one epitope of each of the two or more neoantigens, and the second protein comprises at least one epitope of each of the two or more neoantigens. In some embodiments, the first protein encoded by the first transgene, the second protein encoded by the second transgene, or both include at least one proteasomal cleavage site. In certain embodiments, the first protein encoded by the first transgene, the second protein encode by the second transgene, or both are a fusion protein.

In some embodiments, the method further comprises administering the subject a third boost comprising (i) a dose of third composition comprising a third oncolytic virus that comprises a genome comprising a third transgene wherein the third transgene encodes and expresses a third protein in the subject, wherein the third protein or a fragment thereof is capable of inducing an immune response to the at least one neoantigen, (ii) a dose of fourth composition comprising a fourth oncolytic virus and a first peptide composition, wherein the first peptide composition is capable of inducing an immune response to the at least one neoantigen, or (iii) a dose of a fifth composition and a dose of a sixth composition, wherein the fifth composition comprises the fourth oncolytic virus, and the sixth composition comprises the first peptide composition, and wherein the third oncolytic virus and the fourth oncolytic virus are immunologically distinct from the second oncolytic virus. In a specific embodiment, the third oncolytic virus and the fourth oncolytic virus are immunologically distinct from the first oncolytic virus.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In another aspect, provided herein is a method of inducing an immune response to at least one neoantigen in a subject, comprising: (a) administering to the subject a dose of a priming composition that is capable of inducing an immune response to the at least one neoantigen; (b) subsequently administering to the subject a first boost comprising a dose of a first composition, wherein the first composition comprises a first oncolytic virus that comprises a genome comprising a first transgene, wherein the first transgene encodes and expresses a first protein in the subject, and wherein the first protein or a fragment thereof is capable of inducing an immune response to the at least one neoantigen; and (c) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

In some embodiments, the first protein or fragment thereof and the second protein or fragment thereof are each capable of inducing an immune response to two or more different neoantigens. In certain embodiments, the first protein comprises at least one epitope of each of the two or more neoantigens, and the second protein comprises at least one epitope of each of the two or more neoantigens. In some embodiments, the first protein encoded by the first transgene, the second protein encoded by the second transgene, or both include at least one proteasomal cleavage site. In certain embodiments, the first protein encoded by the first transgene, the second protein encode by the second transgene, or both are a fusion protein.

In some embodiments, the method further comprises administering the subject a third boost comprising (i) a dose of third composition comprising a third oncolytic virus that comprises a genome comprising a third transgene wherein the third transgene encodes and expresses a third protein in the subject, wherein the third protein or a fragment thereof is capable of inducing an immune response to the at least one neoantigen, (ii) a dose of fourth composition comprising a fourth oncolytic virus and a first peptide composition, wherein the first peptide composition is capable of inducing an immune response to the at least one neoantigen, or (iii) a dose of a fifth composition and a dose of a sixth composition, wherein the fifth composition comprises the fourth oncolytic virus, and the sixth composition comprises the first peptide composition, and wherein the third oncolytic virus and the fourth oncolytic virus are immunologically distinct from the second oncolytic virus. In a specific embodiment, the third oncolytic virus and the fourth oncolytic virus are immunologically distinct from the first oncolytic virus.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In another aspect, provided herein is a method of inducing an immune response to at least one neoantigen in a subject, comprising: (a) administering to the subject a dose of a priming composition that is capable of inducing an immune response to the at least one neoantigen; (b) subsequently administering to the subject a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and (c) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

In certain embodiments, the first oncolytic virus comprises a genome that comprises a second transgene, wherein the second transgene encodes and expresses a second protein in the subject, wherein the second protein or a fragment thereof is capable of inducing an immune response to the at least one neoantigen. In certain embodiments, wherein the first protein or fragment thereof is capable of inducing an immune response to two or more different neoantigens. In some embodiments, the first peptide comprises at least one epitope of each of the two or more neoantigens. In certain embodiments, the first protein encoded by the first transgene includes at least one proteasomal cleavage site. In some embodiments, the first protein encoded by the first transgene is a fusion protein. In certain embodiments, the first peptide composition is capable of inducing an immune response to two or more different neoantigens. In some embodiments, the first peptide composition comprises two peptides, wherein one of the peptides comprises at least one epitope of one of the neoantigens, and the other peptide comprises at least one epitope of the other neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In a specific embodiment, the third oncolytic virus and the fourth oncolytic virus are immunologically distinct from the first oncolytic virus.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In certain embodiments, the amino acid sequence of the second peptide composition is different from the amino acid sequence of first protein, the first peptide composition, or both. In other embodiments, the amino acid sequence of the second peptide composition is identical to the amino acid sequence of first protein, the first peptide composition, or both. In some embodiments, the amino acid sequence of the second peptide composition includes at least one epitope found in the first peptide composition.

In another aspect, provided herein is a method of inducing an immune response to at least one neoantigen in a subject, comprising: (a) administering to the subject a dose of a priming composition that is capable of inducing an immune response to the at least one neoantigen; (b) subsequently administering to the subject a first boost comprising a dose of a first composition, wherein the first composition comprises a first oncolytic virus that comprises a genome comprising a first transgene, wherein the first transgene encodes and expresses a first protein in the subject, wherein the first protein or a fragment thereof is capable of inducing an immune response to the at least one neoantigen; and (c) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, and wherein the third and fourth compositions are administered concurrently or sequentially to the subject, and wherein the second oncolytic virus is immunologically distinct from the first oncolytic virus.

In certain embodiments, the second oncolytic virus comprises a genome that comprises a second transgene, wherein the second transgene encodes and expresses a second protein in the subject, wherein the second protein or a fragment thereof is capable of inducing an immune response to the at least one neoantigen. In certain embodiments, wherein the first protein or fragment thereof is capable of inducing an immune response to two or more different neoantigens. In some embodiments, the first peptide comprises at least one epitope of each of the two or more neoantigens. In certain embodiments, the first protein encoded by the first transgene includes at least one proteasomal cleavage site. In some embodiments, the first protein encoded by the first transgene is a fusion protein. In certain embodiments, the first peptide composition is capable of inducing an immune response to two or more different neoantigens. In some embodiments, the first peptide composition comprises two peptides, wherein one of the peptides comprises at least one epitope of one of the neoantigens, and the other peptide comprises at least one epitope of the other neoantigen.

In certain embodiments, the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus. In a specific embodiment, the third oncolytic virus and the fourth oncolytic virus are immunologically distinct from the first oncolytic virus.

The proteins and peptide compositions used in the methods described herein may comprises amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different from the amino acid sequence of first protein, the first peptide composition, or both. In other embodiments, the amino acid sequence of the second protein is identical to the amino acid sequence of first protein, the first peptide composition, or both. In some embodiments, the amino acid sequence of the second protein includes at least one epitope found in the first protein.

In certain embodiments, the amino acid sequence of the second peptide composition is different than the amino acid sequence of first protein, the first peptide composition, or both. In other embodiments, the amino acid sequence of the second peptide composition is identical to the amino acid sequence of first protein, the first peptide composition, or both. In some embodiments, the amino acid sequence of the second peptide composition includes at least one epitope found in the first peptide composition.

In specific embodiments of the methods described herein, one, two or more of the compositions are administered to the subject intravenously or intramuscularly. In certain embodiments, a composition described herein further comprises an adjuvant. In some embodiments, a composition further comprises a liposome or a nanoparticle. In certain embodiments, a composition described herein further comprises an adjuvant and a liposome or nanoparticle.

In some embodiments of the methods described herein, the first boost is administered to the subject 7 to 21 days after the priming composition. In other embodiments of the methods described herein, the first boost is administered to the subject 2 weeks to 3 months after the priming composition.

In some embodiments of the methods described herein, the second boost is administered to the subject 7 to 21 days after the first boost. In other embodiments of the methods described herein, the second boost is administered to the subject 2 weeks to 3 months after the first boost.

In specific embodiments of the methods described herein, the immune response to the at least one neoantigen that is induced in the subject comprises a peak immune response to the at least one neoantigen with the second boost that is at least 0.5 log higher than the peak immune response to the at least one neoantigen attained with the first boost. In specific embodiments of the methods described herein, one month after the second boost the immune response to the at least one neoantigen remains higher that the peak immune response to the at least one neoantigen attained with the first boost. The immune response may be measured by the number of antigen-specific interferon gamma-positive CD8+ T cells per ml of peripheral blood from the subject.

In certain embodiments of the method described herein, the priming composition comprises: (i) a nucleic acid sequence, wherein the nucleic acid sequence encodes and expresses a first priming protein in the subject, wherein the first priming protein or a fragment thereof is capable of inducing an immune response to the at least one neoantigen, (ii) a priming peptide composition, wherein the priming peptide composition is capable of inducing an immune response to the at least one neoantigen, (iii) an adoptive cell transfer of CD8+ T cells specific for the at least one neoantigen, (iv) a first priming virus that comprises a genome comprising a first priming transgene, wherein the first priming transgene encodes and expresses a second priming protein in the subject, wherein the second priming protein or a fragment thereof is capable of inducing an immune response to the at least one neoantigen, or (v) a second priming virus and a first priming peptide composition, and wherein the first priming virus and the second priming virus are immunologically distinct from the first oncolytic virus. In a specific embodiment, the first priming virus and the second priming virus are immunologically distinct from the second oncolytic virus.

In some embodiments of the methods described herein, the first oncolytic virus, the second oncolytic virus, or both are attenuated. In certain embodiments of the methods described herein, the first or second oncolytic virus is a rhabdovirus. In some embodiments of the methods described herein, the first or second oncolytic virus is a Maraba virus (e.g., MG1), a Farmington virus, an adenovirus, a measles virus or a vesicular stomatitis virus.

In certain embodiments of the methods described herein, the first oncolytic virus is a Farmington virus and the second oncolytic virus is a Maraba virus. In other embodiments of the methods described herein, the first oncolytic virus is a Maraba virus and the second oncolytic virus is a Farmington virus. In a specific embodiment, the Maraba virus is MG1.

In certain embodiments of the methods described herein, the first or second oncolytic virus is a vaccinia virus. In some embodiments of the methods described herein, the first oncolytic virus is a vaccinia virus and the second oncolytic virus is a Maraba virus (e.g., MG1). In specific embodiments of the methods described herein, the first oncolytic virus is a Maraba virus (e.g., MG1) and the second oncolytic virus is a vaccinia virus.

In certain embodiments of the methods described herein, the first oncolytic virus is a vaccinia virus and the second oncolytic virus is a Farmington virus. In other embodiments of the methods described herein, the first oncolytic virus is a Farmington virus and the second oncolytic virus is a vaccinia virus. In a specific embodiment, the vaccinia virus is Copenhagen, Western Reserve, Wyeth, Tian Tan or Lister.

In certain embodiments of the methods described herein, a dose of an oncolytic virus is 10⁷ to 10¹² PFU. In some embodiments of the methods described herein, the subject is a mammal. In specific embodiments of the methods described herein, the subject is a human.

### BRIEF DESCRIPTION OF THE DRAWINGS

FIGS. 1A-1B. FIG. 1A Oncolytic rhabdovirus vaccines can boost CD8+ T cell responses against multiple encoded neoantigen targets. C57BL/6 naive mice were primed twice with liposome-wrapped peptides (for 5×MC-38 and 5×B16.F10 tumour neoantigens) IP or PBS (negative control) on days 0 and 7. Mice subsequently received a boost with 3×10⁸ PFU MG1-N10 IV (A) on day 20. Immune responses were analyzed on day 27 (seven days following the boost) after ex-vivo individual peptide stimulation of PBMCs isolated from vaccinated mice. Mean and SEM values are presented. FIG. 1B. C57BL/6 naive mice were primed with peptides (for 5×MC-38 and 5×B16.F10 tumour neoantigens) and adjuvant (anti-CD40 antibody and poly I:C) SC or PBS (negative control) on day 0. Mice subsequently received a boost with 3×10⁸ PFU FMT-N10 IV on day 14. Immune responses were analyzed on day 20 (six days following the boost) after ex-vivo individual peptide stimulation of PBMCs isolated from vaccinated mice. Mean and SEM values are presented.

FIGS. 2A-2B. Oncolytic rhabdovirus vaccines can superboost CD8+ T cell responses against multiple encoded neoantigen targets. C57BL/6 naive mice were primed twice with liposome-wrapped peptides (for 5×MC-38 and 5×B16.F10 tumour neoantigens) IP or PBS (negative control) on days 0 and 7. Mice subsequently received a boost with 3×10⁸ PFU MG1-N10 IV on day 20, and a superboost with 3×10⁸ PFU FMT-N10 IV on day 62. Immune responses were analyzed on day 27 (seven days following boost #1) (FIG. 2A) or day 69 (seven days following boost #2) (FIG. 2B) after ex-vivo individual peptide stimulation of PBMCs isolated from vaccinated mice. Mean and SEM values are presented.

FIG. 3. A boost can engage neoantigen-specific CD8+ T cells established by adenovirus vaccine priming technologies. C57BL/6 mice were primed with rHuAd5-MC7 (encoding 5×MC-38 tumour neoantigens) (2×10⁸ PFU IM) on day 1, and boosted with MG1-MC7 (encoding the same 5×MC-38 tumour neoantigens) (3×10⁸ PFU IV) on day 10. Non-terminal peripheral blood samples were sampled, stimulated with each of the corresponding individual neoantigen peptides, and analyzed by intracellular cytokine staining on day 15 (five days following the boost). Based on five mice per group.

FIGS. 4A-4B. The superboost can engage CD8+ T cells established by multiple nanoparticle priming technologies. Naive mice were primed twice with either liposome-wrapped peptide nanoparticles (for 5×MC-38 and 5×B16.F10 tumour neoantigens), liposome-wrapped mRNA nanoparticles (for 5×MC-38 and 5×B16.F10 tumour neoantigens) or PBS (no prime negative control) on day 0 and 7, followed by an MG1-N10 boost (3×10⁸ PFU IV on day 20) and a FMT-N10 superboost (3×10⁸ PFU IV on day 62). Non-terminal peripheral blood samples were sampled, stimulated with the corresponding 10 neoantigen peptides, and analyzed by intracellular cytokine staining on day 27 (seven days following boost #1) (FIG. 4A) or day 69 (seven days following the superboost #2) (FIG. 4B).

FIGS. 5A-5C. Boosting responses against multiple neoantigen targets does not require a formal prime. Naive mice received vehicle (PBS) followed by MG1-N10 and FMT-N10 boosts. Non-terminal peripheral blood samples were sampled, stimulated with the corresponding 10 neoantigen peptides, and analyzed by intracellular cytokine staining. FIG. 5A shows the experimental protocol and timeline; FIG. 5B shows the ICS results of blood sampling on day 7 (seven days following boost #1). FIG. 5C shows the ICS results of blood sampling on day 49 (seven days following boost #2).

FIG. 6. Three different strategies can be used for encoding multiple neo-antigens to induce antigen-specific CD8 T cell response. C57BL/6 mice were primed twice with ten adjuvanted (with anti-CD40 antibody and poly LC) neoantigen peptides (for 5×MC-38 and 5×B16.F10 tumour neoantigens; IP or SC on day 0 and 7) or PBS (as a negative control), followed by a single boost with MG1-N10, MG1-N10 fusion (where peptides are fused together into a single open reading frame with no intervening sequences) or MG1-N10-opt (where peptides are rationally designed at specific positions in the single open reading frame) (3×10⁸ PFU IV) on day 20. Immune responses following ex vivo peptide stimulation and ICS were measured on day 27. In each treatment group, PBMCs from 10 mice were pooled into 3 biological replicates (3+3+4 mice). Statistics were calculated using the One way ANOVA Kruskal-Wallis test with Dunn's multiple comparison test (* p-value <0.05, ** p-value <0.01, *** p-value <0.001 and **** p-value <0.0001).

FIGS. 7A-7B. Empty oncolytic rhabdovirus vaccines (without a genetically encoded neoantigen transgene cassette) can boost or superboost CD8+ T cell responses against multiple neoantigen targets when administered with loose peptides. C57BL/6 mice were primed with 50 μg of each individual peptide (i.e. five MC-38 tumour neoantigens) plus 10 μg Poly I:C and 30 μg anti-CD40 or PBS (negative control) subcutaneously (SQ or SC) on day 0. Mice were boosted on day 14 with 3×10⁸ PFU FMT-NR IV plus 40 μg of each individual peptide IV or 100 μg of each individual peptide SC. Mice were superboosted on day 28 with 3×10⁸ PFU MG1-NR IV plus 40 μg of each individual peptide IV or 100 μg of each individual peptide SQ. Immune responses were analyzed on day 20 (six days following boost #1) (FIG. 7A) or day 34 (six days following boost #2) (FIG. 7B) after ex-vivo individual peptide stimulation of PBMCs isolated from vaccinated mice. Mean and SEM values are presented. Based on five mice per group. Statistics were calculated using the One way ANOVA Kruskal-Wallis test with Dunn's multiple comparison test (* p-value <0.05, ** p-value <0.01, *** p-value <0.001 and **** p-value <0.0001).

FIG. 8. Oncolytic rhabdovirus vaccines can boost CD8+ T cell responses against multiple encoded neoantigen targets. This figure shows the numbers of CD8+ IFN-γ positive cells of CD8+ T cells obtained following a prime with loose peptides (N10) adjuvanted with anti-CD40 antibody and poly I:C on day 0 and a boost with PBS or 3×10⁸ PFU of FMT N10 (FMT encoding 10 peptides) on day 14. Blood sample was collected on day 20 (six days post boost) and immune response was analysed by intracellular cytokine assay following ex-vivo stimulation of PBMCs with individual minimal CD8 epitopes corresponding to encoded neo-antigens. Statistics were calculated using the t-test Mann-Whitney (* p-value <0.05, ** p-value <0.01, *** p-value <0.001 and **** p-value <0.0001).

FIG. 9. Oncolytic rhabdovirus vaccines can superboost CD8+ T cell responses against multiple encoded neoantigen targets. This figure shows the numbers of CD8+ IFN-γ positive cells of CD8+ T cells obtained at day 34 after mice were primed with loose peptides (N10) adjuvanted with anti-CD40 antibody and poly I:C on day 0, administered a first boost with PBS or 3×10⁸ PFU of FMT N10 (FMT encoding 10 peptides) on day 14, and administered a second boost with 3×10⁸ PFU of MG1 N10 (MG1 encoding 10 peptides) on day 28. Blood sample was collected on day 34 (six days post boost) and immune response was analysed by intracellular cytokine assay following ex-vivo stimulation of PBMCs with individual minimal CD8 epitopes corresponding to encoded neo-antigens. Statistics were calculated using the t-test Mann-Whitney (* p-value <0.05, ** p-value <0.01, *** p-value <0.001 and **** p-value <0.0001).

FIG. 10. Immune responses induced by superboost with empty oncolytic rhabdovirus vaccines (without a genetically encoded neoantigen transgene cassette) administered with loose peptides are maintained at high levels over time. This figure shows the percentage of CD8+ IFN-γ positive cells of CD8+ T cells obtained 30 days after mice received a second boost with 3×10⁸ PFU of MG1 nr plus MC38 SC or MC38 IV. Mice were primed with np or adjuvanted MC38 subcutaneously (SC), administered a first boost with PBS or 3×10⁸ PFU of FMT nr plus MC38 IV on day 14, and administered a second boost with 3×10⁸ PFU of MG1 nr plus MC38 IV on day 28. Statistics were calculated using the One way ANOVA Kruskal-Wallis test with Dunn's multiple comparison test (* p-value <0.05, ** p-value <0.01, *** p-value <0.001 and **** p-value <0.0001).

FIGS. 11A-11B. Empty oncolytic rhabdovirus vaccines (without a genetically encoded neoantigen transgene cassette) can boost or superboost CD8+ T cell responses against multiple neoantigen targets when administered with loose peptides. C57BL/6 mice were primed with 50 μg of each individual peptide (i.e. five B16 tumour neoantigens) plus 10 μg Poly I:C and 30 μg anti-CD40 or PBS (negative control) SC on day 0. Mice were boosted on day 14 with 3×10⁸ PFU FMT-NR IV plus 40 μg of each individual peptide IV. Mice were superboosted on day 28 with 3×10⁸ PFU MG1-NR IV plus 40 μg of each individual peptide IV. Immune responses were analyzed on day 20 (six days following boost #1) (FIG. 11A) or day 34 (six days following boost #2) (FIG. 11B) after ex-vivo individual peptide stimulation of PBMCs isolated from vaccinated mice. Mean and SEM values are presented.

FIGS. 12A-12C. Boosting responses against multiple neoantigen targets does not require a formal prime. Naïve mice received vehicle (PBS) followed by FMT-N10 and MG1-N10 boosts. Non-terminal peripheral blood samples were sampled, stimulated with the corresponding 10 neoantigen peptides, and analyzed by intracellular cytokine staining. FIG. 12A shows the experimental protocol and timeline; FIG. 12B shows the ICS results of blood sampling on day 6 (six days following boost #1) and FIG. 12C shows the ICS results of blood sampling on day 20 (six days following boost #2). Statistics were calculated using the t-test Mann-Whitney (* p-value <0.05, ** p-value <0.01, *** p-value <0.001 and **** p-value <0.0001).

FIG. 13. A boost can engage CD8+ T cells established by mRNA nanoparticle priming technology. Naive mice were primed twice with liposome-wrapped mRNA nanoparticles (for 5×MC-38 and 5×B16.F10 tumour neoantigens) or PBS (no prime negative control) on day 0 and 7, followed by an MG1-N10 boost (3×10⁸ PFU IV on day 20). Non-terminal peripheral blood samples were sampled, stimulated with the corresponding 10 neoantigen peptides, and analyzed by intracellular cytokine staining on day 27 (seven days following boost #1). Statistics were calculated using the t-test Mann-Whitney (* p-value <0.05, ** p-value <0.01, *** p-value <0.001 and **** p-value <0.0001).

### DETAILED DESCRIPTION

### Neoantigens

In one aspect, provided herein are methods for inducing an immune response to one or more neoantigens. In a specific embodiment, neoantigens are mutated, non-self products that arise from some tumor accumulated genetic alterations. The inherent genetic instability of cancers can lead to mutations in DNA, RNA splice variants and changes in post-translational modification, which result in these de novo mutated, non-self protein products. These mutated protein products may be processed, presented by human leukocyte antigen (HLA) molecules and elicit T-cell responses to these tumor-specific somatic mutations. The mutated protein products are specific to tumor cells and are often but not always unique to an individual subject.

Generally, cancer patients have a tumor with a unique combination of neoantigens (sometimes referred to herein as “private neoantigens”). The term “mutanome” may be used herein to refer to the collective of a subject's tumor-specific mutations, which encode a set of neoantigens that are specific to the subject. See, e.g., Tureci et al., Clin Cancer Res. 2016; 22(8):1885-1896. The mutanome can readily be determined for a given tumor, e.g., by next generation sequencing.

In specific embodiments, a neoantigen is a tumor-associated antigen that is subject-specific, and is sometimes referred herein to as a “private neoantigen.” In other embodiments, a neoantigen appears across a patient population, and is sometimes referred to herein as a “public neoantigen.” For example, mutations that alter protein function to promote oncogenesis, so-called driver mutations, can systematically reappear across patients. See, e.g., Kiebanoff and Wolchok, 2017, J Exp. Med., 215(1):5-7. Non-limiting examples of public neoantigens include mutated KRAS, such as KRAS G12D (see, e.g., Tran et al., 2016, N. Engl. J. Med. 375: 225-2262) and KRAS G12V (see, e.g., Veatech et al., 2019, Cancer Immunol. Res. 7: 910-922), mutated p53, such as p53 p.R175H (see, e.g., Lo et al., 2019, Cancer Immunol. Res. 7: 534-543), and mutated histone, such as histone variant H3.3 (H3.3K27M) (see, e.g., Mackay et al., Cancer Cell 32: 520-537), and mutated calreticulin (see, e.g,. Bozkus et al., 2019, Cancer Discov. 9: 1-6). Public neoantigens may be used to develop targeted immunotherapy approaches applicable to significant patient populations in contrast to private neoantigens, which generally require next generation sequencing and complex algorithms.

Neoantigens may arise from DNA mutations including, e.g., nonsynonymous missense mutations, nonsense mutations, insertions, deletions, chromosomal inversions and chromosomal translocations. Neoantigens may arise from RNA splice site changes or missense mutations that can introduce amino acids permissive to post-translational modifications (e.g., phosphorylation). In certain embodiments, neoantigens may be created by one, two, three or more of the following or a combination thereof: (1) nucleotide polymorphisms that result in non-conservative amino acid changes; (2) insertions and/or deletions, which can result in peptide antigens containing an insertion or deletion or a frameshift mutation; (3) the introduction of a stop codon that in its new context is not recognized by the stop codon machinery, resulting in the ribosome skipping the codon and generating a peptide that contains a single amino acid deletion; (4) mutations at splice sites, which result in incorrectly spliced mRNA transcripts; and (5) inversions and/or chromosomal translocations that result in fusion peptides.

In some embodiments, a process is used to select the one or more neoantigens to which to induce an immune response. Neoantigens may be prioritized according to their MHC binding affinity and RNA expression levels within tumor cells. For example, neoantigens may be prioritized according to their predicted MHC class I binding, their MHC class II binding, or both. See, e.g., Kreiter et al., 2015, Nature 520: 692-696 and Yadav et al., 2014, Nature 515: 572-578 for methods for predicting MHC binding of neoantigens. In some embodiments, additional criteria are applied, such as, e.g., predicted immunogenicity or predicted capacity of the neoantigen to lead to T cells that react with other self-antigens, which may lead to auto-immunity. In some embodiments, neoantigens that are predicted to result in T cell or antibody responses that react with self-antigens found on healthy cells are not selected for use in the methods described herein.

In a specific embodiment, a peptide or protein that is capable of inducing an immune response to a neoantigen is selected for use in a method described herein. The terms peptide or polypeptide may be used interchangeably herein to refer to ae natural or non-natural amino acid sequence. The peptide or polypeptide may or may not contain post-translational modifications, such as, e.g., glycosylation, phosphorylation or both. As used herein, a peptide or protein that is capable of inducing an immune response to a neoantigen of interest may be referred to as an “antigenic protein,” whether in the context of a prime or a boost.

In some embodiments, a process is used to select a peptide or protein that is capable of inducing an immune response to one or more neoantigens. For example, the peptide or protein may be assessed for its MHC binding affinity, its structural similarity to a neoantigen, or both. In some embodiments, a peptide or protein is selected that is at least about 70% identical, at least about 80% identical, at least about 90% identical, at least about 95% identical to a particular neoantigen. In some embodiments, a peptide or protein is selected that is identical to a particular neoantigen. In some embodiments, a peptide or protein is selected that is structurally or conformationally similar to a particular neoantigen as assessed using a method to known one of skill in the art, such as, e.g., NMR, X-ray crystaollographic methods, or secondary structure prediction methods, such as, e.g., circular dichroism. In a particular embodiment, a peptide or protein with the highest predicted MHC class I binding, MHC class II binding, or both may be selected to induce an immune response to one or more neoantigens. See, e.g., Kreiter et al., 2015, Nature 520: 692-696 and Yadav et al., 2014, Nature 515: 572-578 for methods for predicting MHC binding. In certain embodiments, a peptide or protein is selected for use in a method of inducing an immune response that is predicted to elicit a CD4 T cell response, a CD8 T cell response, or both. In some embodiments, a peptide or protein is selected for use in a method of inducing an immune response that contains a CD4 epitope. In certain embodiments, a peptide or protein is selected for use in a method of inducing an immune response that contains a CD8 epitope. In some embodiments, additional criteria are applied in the selection of a peptide or protein that is capable of inducing an immune response to a neoantigen, such as, e.g., predicted immunogenicity or predicted capacity of the peptide or protein to lead to T cells that react with other self-antigens, which may lead to auto-immunity. In some embodiments, peptides or proteins that are predicted to result in T cell or antibody responses that react with self-antigens found on healthy cells are not selected for use in the methods described herein.

The term “about,” as used herein refers to plus or minus 10% of a reference, e.g., a reference amount, time, length, or activity. In instances where integers are required or expected, it is understood that the scope of this term includes rounding up to the next integer and rounding down to the next integer. In instances where the reference is measured in terms of days, the scope of this term also includes plus or minus 1, 2, 3, or 4 days. For clarity, use herein of phrases such as “about X,” and “at least about X,” are understood to encompass and particularly recite “X.”

The determination of percent identity between two amino acid sequences may be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the XBLAST program of Altschul et al, 1990, J. Mol. Biol. 215:403. BLAST protein searches may be performed with the XBLAST program parameters set, e.g., to score 50, word length=3 to obtain amino acid sequences homologous to a protein molecule described herein. To obtain gapped alignments for comparison purposes, Gapped BLAST may be utilized as described in Altschul et al, 1997, Nucleic Acids Res. 25:3389 3402. Alternatively, PSI BLAST may be used to perform an iterated search which detects distant relationships between molecules (Id.). When utilizing XBLAST, the default parameters of the program may be used (see, e.g., National Center for Biotechnology Information (NCBI), ncbi.nlm.nih.gov). Another non limiting example of a mathematical algorithm utilized for the comparison of sequences is the algorithm of Myers and Miller, 1988, CABIOS 4: 11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. The percent identity between two sequences may be determined using techniques similar to those described above, with or without allowing gaps. In calculating percent identity, typically only exact matches are counted.

In a specific embodiment, an antigenic protein that is identical to a neoantigen or a fragment thereof (e.g., a portion of the neoantigen that contains an epitope) is selected for use in the methods described herein. In one embodiment, the fragment of the neoantigen is at least 8 amino acids in length, and in some embodiments, the fragment is about 8 to about 15 amino acids in length, about 12 to about 15 amino acids in length, about 15 to about 25 amino acids in length, about 25 to 30 amino acids in length, about 25 to about 50 amino acids in length, about 25 to about 75 amino acids in length, or about 50 to about 75 amino acids in length. In some embodiments, the fragment of the neoantigen is about 50 to about 100 amino acids in length, about 75 to about 100 amino acids in length, about 75 to about 125 amino acids in length, about 100 to about 125 amino acids in length, about 125 to about 150 amino acids in length, about 100 to about 150 amino acids in length, about 150 to about 200 amino acids in length, about 8 to about 250 amino acids in length, or about 150 to about 300 amino acids in length. The antigenic protein that is used in the methods described herein may contain a CD4 epitope, a CD8 epitope, or both.

In certain embodiments, at least one antigenic protein of a composition (e.g., a priming composition, boosting composition, or both) containing one or more antigenic proteins ranges in length from about 8 to about 500 amino acids. For example, at least one antigenic protein may be at least about 8, at least about 10, at least about 20, at least about 25, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In some embodiments, one or more of the antigenic proteins may be synthetic proteins. In certain embodiments, one or more antigenic proteins may be recombinant proteins.

In certain embodiments, an antigenic protein is about 8 to about 500 amino acids in length, about 25 to about 500 amino acids in length, about 25 to about 400 amino acids in length, about 25 to about 300 amino acids in length, about 25 to about 200 amino acids in length, or about 25 to about 100 amino acids in length, and contains at least a fragment (e.g., an epitope) of at least one neoantigen of interest. In some embodiments, an antigenic protein is about 25 to about 250 amino acids in length, about 25 to about 75 amino acids in length, or about 25 to about 50 amino acids in length, and contains at least a fragment (e.g., an epitope) of at least one neoantigen of interest. In some embodiments, an antigenic protein about 250 to about 1000 amino acids in length, about 250 to about 750 amino acids in length, or about 250 to about 500 amino acids in length, and contains at least a fragment (e.g., an epitope) of at least one neoantigen of interest. Any combination of the stated upper and lower limits is also envisaged.

In certain embodiments, an antigenic protein that is used in a method of inducing an immune response described herein contains at least a fragment (e.g., an epitope) of one or more neoantigens of interest. Thus, in some embodiments, an antigenic protein that is used in a method of inducing an immune response described herein contains at least a fragment (e.g., an epitope) of 2, 3, 4, 5, 6, 7, 8, 9, 10 or more neoantigens of interest. In certain embodiments, an antigenic protein that is used in a method of inducing an immune response described herein contains at least a fragment (e.g., an epitope) of 2 to 20, 2 to 15, 2 to 10, 5 to 10, 15 to 20, or 2 to 5 neoantigens of interest. In some embodiments, the antigenic protein that is used in a method of inducing an immune response described herein contains at least two neoantigens or a fragment of each of the at least two neoantigens. In certain embodiments, the at least two neoantigens are public neoantigens. The appropriate combination of public neoantigens to be administered may be determined by a simple diagnostic test, such as, e.g., RT-PCR or through an ELISA immunoassay. In other embodiments, the at least two neoantigens are private neoantigens. In some embodiments, one of the least two neoantigens in a private neoantigen and the other of the least neoantigens is a public neoantigen. In other words, in some embodiments, an antigenic protein may comprises a mix of both public and private neoantigens.

In a specific embodiment, an antigenic protein is a fusion protein comprising 2 or more neoantigens or fragments (e.g., an epitope) of each of the 2 or more neoantigens. In certain embodiments, the fusion protein includes spacers, cleavage sites (e.g., proteosomal cleavage sites, such as, e.g., described in Section 6), or both. See, e.g., Schubert and Kohlbacher, 2016, Genome Medicine 8: 9 for techniques for designing antigenic proteins with optimal spacers.

In certain embodiments, an antigenic protein is a fusion protein comprising two or more neoantigens or fragments thereof, and the two neoantigens or fragments thereof are randomly ordered in the fusion protein. In some embodiments, an antigenic protein is a fusion protein comprising two or more neoantigens or fragments thereof, and the two neoantigens or fragments thereof are ordered 5′ to 3′ in the fusion protein on the basis of the predicted MHC binding affinity of the two or more neoantigens or fragments thereof. In certain embodiments, the neoantigen or fragment thereof with the highest predicted MHC binding affinity is first in the fusion protein. In other embodiments, the neoantigen or fragment thereof with the lowest predicted MHC binding affinity is last in the fusion protein. In a specific embodiment, a technique as described in Section 6, infra, is used to optimize the order of two or more neoantigens or fragments thereof in a fusion protein.

### Priming Compositions

In one aspect, provided herein are compositions for use as a prime in the methods presented herein. In a specific embodiment, provided herein are priming compositions that may be used in the methods presented herein. In a specific embodiment, a priming composition is capable of and is used to induce an immune response to one or more neoantigens in a subject. In certain embodiments, a priming composition is used to induce an immune response to 2 to about 20 neoantigens. In some embodiments, a priming composition is used to induce an immune response to 2, 3, 4, 5, 6, 7, 8, 9, or 10 neoantigens in a subject. In certain embodiments, a priming composition is used to induce an immune response to 1 to 3, 1 to 5, 2 to 4, 2 to 5, 2 to 6, 2 to 8, 5 to 8, 5 to 10, or 8 to 10 neoantigens in a subject. Any combination of the stated upper and lower limits is also envisaged. In a specific embodiment, a priming composition is one described in Section 6, infra, to prime a subject.

In one embodiment, a priming composition comprises an adoptive cell transfer of CD8+ T cells specific for at least one neoantigen. In a specific embodiment, the antigen-specific CD8+ T cells of the adoptive transfer may be native or engineered antigen-specific CD8+ T cells.

In another embodiment, a priming composition comprises a nucleic acid-based priming agent, e.g., an RNA priming agent. In a specific embodiment, a priming composition comprises a nucleic acid sequence (e.g., an RNA sequence or cDNA sequence), wherein the nucleic acid sequence encodes and expresses a protein in the subject, wherein the protein or a fragment thereof is capable of inducing an immune response to at least one neoantigen. A “nucleic acid” or “nucleic acid sequence” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded. In various embodiments, the nucleic-acid based priming agent may be delivered through an expression vector, or delivered through non-vector based methods known in the art. Such vectors may include a viral vector, an non-viral vector (e.g., a plasmid) or loaded antigen-presenting cell such as a dendritic cell. In a specific embodiment, if a viral vector is used to deliver a nucleic acid-based priming agent, the viral vector is immunological distinct from the first post-priming boost. In some embodiments, if a viral vector is used to deliver a nucleic acid-based priming agent, the viral vector is immunological distinct from the first and second post-priming boosts. In certain embodiments, if a viral vector is used to deliver a nucleic acid-based priming agent, the viral vector is immunological distinct from each of the post-priming boosts. In specific embodiments, a non-viral vector is used to deliver a nucleic acid-based priming agent.

In certain embodiments where a priming composition comprises a nucleic acid sequence(s) that encodes and expresses one or more antigenic proteins, at least one antigenic protein may range in length from about 8 to about 500 amino acids. In particular embodiments, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters.

In certain embodiments, for example, such a nucleic acid sequence(s) that expresses one or more antigenic proteins may encode at least one antigenic protein ranging in length from about 8 to about 500 amino acids. For example, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters. In some embodiments, a nucleic acid sequence comprises a codon-optimized nucleotide sequence encoding an antigenic protein.

In instances where a nucleic acid sequence that encodes more than one antigenic protein, in certain embodiments, the nucleic acid sequence may express the more than one antigenic proteins as a single, larger protein. In instances wherein two or more antigenic proteins are expressed as part of a single, longer protein, in certain embodiments, the portion(s) of the longer protein corresponding to at least one individual antigenic protein fall(s) within these length parameters. In other embodiments, the portions of the longer protein corresponding to each of the individual antigenic proteins fall within these length parameters.

In another embodiment, a priming composition comprises a peptide is capable of inducing an immune response to the at least one neoantigen. In another embodiment, a priming composition comprises a priming virus that comprises a genome comprising a transgene, wherein the transgene encodes and expresses a protein in the subject, wherein the protein or a fragment thereof is capable of inducing an immune response to at least one neoantigen, and wherein the priming virus is immunologically distinct from an oncolytic virus used in a first boost of a method presented herein. In some embodiments, the priming virus is immunologically distinct from an oncolytic virus used in a first boost and a second boost of a method presented herein.

In certain embodiments, a priming virus is immunologically distinct from the oncolytic virus utilized in at least the first post-prime boost in a heterologous method described herein. In some embodiments, a priming virus is immunologically distinct from the oncolytic viruses utilized in each of the boosts in a heterologous boost method described herein.

In another embodiment, a priming composition comprises a first composition and a second composition, wherein the first composition comprises a priming virus, and the second composition comprises a peptide, wherein the peptide or fragment thereof is capable of inducing an immune response to at least one neoantigen, that is an antigenic protein, and wherein the priming virus is immunologically distinct from an oncolytic virus used in a first boost. In some embodiments, the priming virus is immunologically distinct from an oncolytic virus used in a first boost and a second boost of a method presented herein.

In general, two viruses, e.g., two oncolytic viruses, are immunologically distinct when the two viruses do not induce neutralizing antibodies against each other to such a degree that the viruses may no longer deliver antigen to the immune system. In certain embodiments, two viruses, e.g., oncolytic viruses, are immunologically distinct when the viruses do not induce antibodies that substantially inhibit replication of the other as assessed by a virus neutralization assay, such as described in Tesfay et al., 2014, J. Virol. 88: 6148. In a specific embodiment, two viruses are immunologically distinct when one virus induces antibodies that inhibit the replication of the other virus in a virus neutralization assay, e.g., a virus neutralization assay described in Tesfay et al., 2014, J. Virol. 88: 6148, by less than about 0.5 logs, less than about 1 log, less than about 1.5 logs, or less than about 2 logs. With respect to rhabdoviruses, in particular embodiments, for example, two rhabdoviruses are immunologically distinct when the antibodies induced by the G protein of one rhabdovirus inhibit the replication of the rhabdovirus in a virus neutralization, e.g., a virus neutralization assay as described in Tesfay et al., 2014, J. Virol. 88: 6148, by less than about 0.5 logs, less than about 1 log, less than about 1.5 logs, or less than about 2 logs. Non-limiting examples of viruses that are immunologically distinct from each other include non-pseudotyped Farmington virus and Maraba virus (e.g., Maraba MG1 virus). Non-limiting examples of viruses wherein each is immunologically distinct from the other also include non-pseudotyped Farmington virus, Maraba virus (e.g., Maraba MG1 virus), vaccinia virus, and measles virus. Non-limiting examples of viruses wherein each is immunologically distinct from the other also include non-pseudotyped adenovirus, Farmington virus, vesicular stomatitis virus, vaccinia virus, and measles virus.

In certain embodiments, a priming virus comprises a genome that comprises a transgene or a nucleic acid sequence that expresses an antigenic protein. In some embodiments, a priming virus is an adenovirus. In certain embodiments, a priming virus is an oncolytic virus. See, e.g., Section 5.3 and 6, infra, for examples of oncolytic viruses. In some embodiments, the priming virus may be attenuated. For example, in certain embodiments, the priming virus may have reduced virulence, but still be viable or “live.” In specific embodiments, the priming virus is attenuated but replication-competent. In certain embodiments, the priming virus is replication-defective. In certain embodiments, a priming virus is inactivated, (e.g., UV inactivated).

In some embodiments, a priming composition may comprise (i) one or more peptides capable of inducing an immune response to the one or more neoantigens of interest, that is, may comprise one or more antigenic proteins, and (ii) a priming virus that comprises a genome comprising a transgene(s) or nucleic acid sequence(s), wherein the transgene(s) or nucleic acid sequence(s) express one or more proteins capable of inducing an immune response to the one or more neoantigens of interest, that is, express one or more antigenic proteins. In particular embodiments, a priming composition comprises one or more peptides capable of inducing an immune response to a first subset of the neoantigens of interest, and a priming virus that comprises a genome comprising a transgene(s) or nucleic acid sequence(s), wherein the transgene(s) or nucleic acid sequence(s) express one or more proteins capable of inducing an immune response to a second subset of the neoantigens of interest. In some embodiments, the first subset includes public neoantigens of interest and the second subset includes private neoantigens, or vice versa. In certain embodiments, the first subset and the second subset of neoantigens of interest are overlapping subsets. In other embodiments, the first subset and the second subset of neoantigens of interest do not overlap. In yet other embodiments, a priming composition comprises (i) one or more peptides capable of inducing an immune response to the neoantigens of interest, and (ii) a priming virus comprises a genome that comprises transgene(s) or nucleic acid sequence(s), wherein the transgene(s) or nucleic acid sequence(s) expresses one or more proteins capable of inducing an immune response to the neoantigens of interest. In some embodiments, the one or more peptides and the priming virus are administered in the same composition. In other embodiments, the one or more peptides and the priming virus are administered in different compositions. The different compositions may be formulated for administration by the same or different routes of administration.

In some embodiments, a priming virus does not comprise a genome that comprises a nucleic acid sequence or transgene that expresses an antigenic protein. A virus that does not comprise a genome that comprises nucleic acid sequence or transgene that expresses the antigenic protein refers to a virus that does not produce the antigenic protein and does not cause a cell infected by the virus to produce the protein. For example, the priming virus may lack a nucleic acid sequence that encodes the amino acid sequence of the antigenic protein, or lack nucleic acid sequences necessary for the transcription and/or translation required for the virus to express the antigenic protein or to cause a cell infected by the virus to express the antigenic protein. In another example, the priming virus may lack a nucleic acid sequence that encodes the amino acid sequence of the antigenic protein, and lack nucleic acid sequences necessary for the transcription and/or translation required for the virus to express the antigenic protein or to cause a cell infected by the virus to express the antigenic protein. In one embodiment, a priming virus that does not comprise a genome that comprises a transgene or a nucleic acid sequence that expresses the antigenic protein is an adenovirus (e.g., an adenovirus of serotype 5). For example, in one embodiment, an adenovirus is a recombinant replication-incompetent human Adenovirus serotype 5.

In certain embodiments, a priming virus that does not comprise a genome that comprises a transgene or nucleic acid sequence that expresses an antigenic protein may be attenuated. For example, in certain embodiments, the virus of the prime may have reduced virulence, but still be viable or “live.” In specific embodiments, the priming virus is attenuated but replication-competent. In certain embodiments, a priming virus that does not comprise a genome that comprises a transgene or nucleic acid sequence that expresses an antigenic protein is replication-defective. In some embodiments, a priming virus that does not comprise a genome that comprises a transgene or nucleic acid sequence that expresses an antigenic protein is inactivated, (e.g., UV inactivated).

In a particular embodiment, a priming virus is not engineered to (i) contain a transgene or nucleic acid sequence that encodes the amino acid sequence of the antigenic protein, or (ii) contain nucleic acid sequences necessary for the transcription and/or translation required for the virus to express the antigenic protein or to cause a cell infected by the virus to express the antigenic protein. In another embodiment, a priming virus is not engineered to (i) contain a transgene or nucleic acid sequence that encodes the amino acid sequence of the antigenic protein, and (ii) contain nucleic acid sequences necessary for the transcription and/or translation required for the virus to express the antigenic protein or to cause a cell infected by the virus to express the antigenic protein.

In certain embodiments, a priming virus that does not comprise a transgene or nucleic acid sequence that expresses the antigenic protein, the antigenic protein is not physically associated with and/or connected to the virus. For example, in certain embodiments, the antigenic protein (i) is not attached to, conjugated to or otherwise covalent bonded to the priming virus, (ii) does not become attached to, conjugated to or otherwise covalently bonded to the priming virus, (iii) does not non-covalently interact with the priming virus, or (iv) does not form non-covalent interactions with the priming virus. In some embodiments, two, three or all of the following apply to the antigenic protein: (i) the antigenic protein is not attached to, conjugated to or otherwise covalent bonded to the priming virus, (ii) the antigenic protein does not become attached to, conjugated to or otherwise covalently bonded to the priming virus, (iii) the antigenic protein does not non-covalently interact with the priming virus, and (iv) the antigenic protein does not form non-covalent interactions with the priming virus. In other particular embodiments, the antigenic protein is may be physically associated with and/or connected to the virus. For example, in particular embodiments, the antigenic protein (i) may be attached to, conjugated to or otherwise covalent bonded to the virus, (ii) may become attached to, conjugated to or otherwise covalently bonded to the virus, (iii) may non-covalently interact with the virus, or (iv) form non-covalent interactions with the virus. In some embodiments, one, two, three or all of the following apply to the antigenic protein: (i) may be attached to, conjugated to or otherwise covalent bonded to the virus, (ii) may become attached to, conjugated to or otherwise covalently bonded to the virus, (iii) may non-covalently interact with the virus, and (iv) form non-covalent interactions with the virus.

In certain embodiments, a priming composition comprises one or more antigenic proteins. In some embodiments, a priming composition comprises one or more antigenic proteins, wherein at least one antigenic protein ranges in length from about 8 to about 500 amino acids. For example, at least one antigenic protein may be at least about 8, at least about 10, at least about 20, at least about 25, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, one or more of the antigenic proteins of a priming composition may be synthetic proteins. In some embodiments, one or more antigenic proteins of a priming composition may be recombinant proteins.

In certain embodiments, a priming composition comprises an antigenic protein, wherein the antigenic protein may comprise the entire amino acid sequence of the neoantigen of interest. In such embodiments, the antigenic protein may be as long or longer than the neoantigen of interest. In some embodiments, a priming composition comprises an antigenic protein, wherein antigenic protein may comprise an amino acid sequence shorter than the neoantigen of interest, but a minimum of about 8 amino acid residues, about 9 amino acid residues, about 10 amino acid residues, about 11 amino acid residues, or about 12 amino acid residues in length.

In certain embodiments in which a priming composition comprises a priming virus that comprises a genome comprising a transgene, the transgene comprises a nucleic acid sequence that encodes an antigenic protein such that it is expressed in the subject. The transgene may also include additional sequences, such as, e.g., viral regulatory signals (e.g., gene end, intergenic, and/or gene start sequences) and Kozak sequences. Generally, the total length of a transgene is limited only by the nucleic acid carrying capacity of the particular virus, that is, the amount of nucleic acid that can be inserted into the genome of the virus without preventing a sufficient amount of the protein encoded by the transgene to be produced. In specific embodiments, a sufficient amount of the protein encoded by the transgene is enough to induce an immune response to a neoantigen. In certain embodiments, the total length of a transgene is limited only by the nucleic acid carrying capacity of the particular virus, that is, the amount of nucleic acid that can be inserted into the genome of the virus without significantly inhibiting the pre-insertion replication capability of the virus. In some embodiments, the amount of nucleic acid inserted into the genome of a virus does not significantly inhibit the pre-insertion replication capability of the virus if it does not reduce the replication by more than about 0.5 log, about 1 log, about 1.5 log, about 2 logs, about 2.5 logs, or about 3 logs in a particular cell line relative the replication of the virus absent the insert in the same cell line. In particular embodiments, for example, in instances where the virus is a Farmington virus or a Maraba virus, for example an MG1 virus, a transgene of about 3-5 kb, e.g., about 3 kb, about 3.5 kb, about 4 kb, about 4.5 kb, or about 5 kb, may be inserted into the virus genome. In the case of Maraba virus, e.g., MG1 virus, the nucleic acids expressing the antigenic proteins may, for example, be inserted into the Maraba genome between the G and L gene sequences. In the case of Farmington virus, e.g., FMT virus, the nucleic acids expressing the antigenic proteins may, for example, be inserted into the Farmington genome between the N and P gene sequences. Techniques known in the art may be used to insert a transgene into the genome of a virus and to assess the presence of the inserted transgene in the genome.

In certain embodiments where a priming composition comprises a priming virus that comprises a transgene, wherein the transgene encodes and expresses one or more antigenic proteins in a subject, at least one antigenic protein may range in length from about 8 to about 500 amino acids. In particular embodiments, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters. In some embodiments, a transgene comprises a codon-optimized nucleotide sequence encoding an antigenic protein.

In instances where a transgene encodes and expresses one or more antigenic proteins in a subject, in certain embodiments, the transgene can express the more than one antigenic protein as a single, longer protein. In instances wherein two or more antigenic proteins are expressed as part of a single, longer protein, in certain embodiments, the portion(s) of the longer protein corresponding to at least one individual antigenic protein fall(s) within these length parameters. In other embodiments, the portions of the longer protein corresponding to each of the individual antigenic proteins fall within these length parameters.

In certain embodiments where a transgene encodes and expresses an antigenic protein in a subject, the antigenic protein may comprise the entire amino acid sequence of the neoantigen of interest. In such embodiments, the antigenic protein may be as long or longer than the neoantigen of interest.

In some embodiments, a priming virus comprises a genome that comprises transgene or nucleic acid sequences, wherein the transgene or nucleic acid sequences express x number of antigenic proteins, the genome may comprise a nucleic acid for each of the antigenic proteins, that is, a first nucleic acid that expresses the first antigenic protein, a second nucleic acid that expresses the second antigenic protein, etc., up to and including an xth nucleic acid that encodes the xth antigenic protein. In particular embodiments, the first antigenic protein is capable of inducing an immune response to a first neoantigen, the second antigenic protein is capable of inducing an immune response to a second neoantigen, etc., up to and including the xth antigenic protein being capable of inducing an immune response to an xth neoantigen. In certain embodiments, the transgene or nucleic acid sequences that express x number of antigenic proteins does not prevent a sufficient amount of the protein encoded by the transgene to be produced. In specific embodiments, a sufficient amount of the protein encoded by the transgene is enough to induce an immune response to the xth neoantigen. In a specific embodiment, the transgene or nucleic acid sequences that express x number of antigenic proteins does not significantly inhibit the pre-insertion replication capability of the virus if the transgene or nucleic acid sequence inserted into the viral genome does not reduce the replication of the virus by more than about 0.5 log, about 1 log, about 1.5 log, about 2 logs, about 2.5 logs, or about 3 logs in a particular cell line relative the replication of the virus absent the insert in the same cell line.

Within the virus, a nucleic acid sequence that expresses a particular antigenic protein may be contiguous to or separate from a nucleic acid sequence that expresses a different antigenic protein. In certain embodiments, each of the nucleic acid sequences expressing the antigenic protein may be present in the virus as a transgene. In some embodiments, each of the nucleic acid sequences expressing antigenic proteins is a fusion protein. As noted above, generally, the total length or lengths of such nucleic acid or nucleic acid sequences within the virus need only be limited by the nucleic acid carrying capacity of the virus. In certain embodiments, the nucleic acid sequences may express antigenic proteins as individual proteins. In certain embodiments, nucleic acid sequences may express antigenic proteins together as part of a longer protein. In certain embodiments, nucleic acid sequences may express certain of antigenic proteins as individual proteins and certain of antigenic proteins together as part of a longer protein. In instances where two or more antigenic proteins are expressed as part of a longer protein, the antigenic proteins may be adjacent to each other, with no intervening amino acids between them, or may be separated by an amino acid spacer. See, e.g., Schubert and Kohlbacher, 2016, Genome Medicine 8: 9 for techniques for designing antigenic proteins with optimal spacers. In certain embodiments involving a longer protein, some of antigenic proteins may be adjacent to each other and others may be separated by an amino acid spacer. In certain embodiments, the longer protein comprises one or more cleavage sites, for example, one or more proteasomal cleavage sites. In particular embodiments, the protein comprises one or more amino acid spacers that comprise one or more cleavage sites, for example, one or more proteasomal cleavage sites. See, e.g., Section 6, infra, for examples of nucleic acid sequences encoding one or more antigenic proteins.

In one embodiment, a priming virus is an adenovirus. In a particular embodiment, the adenovirus is of serotype 5. For example, in one embodiment, an adenovirus is a recombinant replication-incompetent human Adenovirus serotype 5. In some embodiments, a priming virus is an oncolytic virus. See, e.g., Section 5.3 and 6, infra, for examples of oncolytic viruses. In certain embodiments, the priming virus may be attenuated. For example, in certain embodiments, the virus of the prime may have reduced virulence, but still be viable or “live.” In some embodiments, the priming virus is inactivated, e.g., the virus is UV inactivated.

In certain embodiments, a priming composition described herein further comprises an adjuvant. In certain embodiments, the adjuvant can potentiate an immune response to an antigen or modulate it toward a desired immune response. In some embodiments, the adjuvant can potentiate an immune response to an antigen and modulate it toward a desired immune response. In one embodiment, the adjuvant is polyI:C.

In some embodiments, an antigenic protein, a nucleic acid sequence expressing an antigenic protein, or a priming virus is not encapsulated in a delivery vehicle such as a liposomal preparation or nanoparticle. In a specific embodiment, an antigenic protein is not encapsulated in a delivery vehicle such as a liposomal preparation or nanoparticle. In another embodiment, a nucleic acid sequence expressing an antigenic protein is not encapsulated in a delivery vehicle such as a liposomal preparation or nanoparticle. In another embodiment, a priming virus is not encapsulated in a delivery vehicle such as a liposomal preparation or nanoparticle.

In some embodiments, a priming composition described herein further comprises a liposome(s) or a nanoparticle. In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate an antigenic protein, a nucleic acid sequence expressing an antigenic protein, or a priming virus. See, e.g., Sahin et al. (2014), mRNA-based therapeutics—developing a new class of drugs. NATURE REVIEWS DRUG DISCOVERY, 13(10):759-780; Su et al. (2011) In vitro and in vivo mRNA delivery using lipid-enveloped pH-responsive polymer nanoparticles, MOLECULAR PHARMACEUTICALS, 8 (3):-774-778; Phua et al., (2014) Messenger RNA (mRNA) nanoparticle tumour vaccination, NANOSCALE, 6(14):7715-7729; Bockzkowski et al., Dendritic cells pulsed with RNA are potent antigen presenting cells in vitro and in vivo, JOURNAL OF EXPERIMENTAL MEDICINE, 184(2):465-472.

In some embodiments, a priming composition described herein further comprises a liposome(s) or a nanoparticle and an adjuvant. In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate (i) an antigenic protein, a nucleic acid sequence expressing an antigenic protein, or a priming virus, and (2) an adjuvant.

In one embodiment, a priming composition is formulated for intravenous, intramuscular, subcutaneous, intraperitoneal or intratumoral administration. When a priming composition is to be administered in parts, different parts of the priming composition may be formulated for the same or different routes of administration. For example, when a priming composition comprises a first composition and a second composition, wherein the first composition comprises a priming virus, and the second composition comprises an antigenic protein, the first composition may be administered by the same or a different route than the second composition. In a particular embodiment, a priming composition is formulated for intravenous administration. In another embodiment, a priming composition is formulated for subcutaneous or intramuscular administration.

In certain embodiments, a priming composition comprises 1×10⁷ to 5×10¹² PFU of a priming virus. For example, in some embodiments, a priming composition comprises 1×10⁷ to 1×10¹² PFU of a priming virus. In certain embodiments, a priming composition comprises about 1×10¹¹ PFU, about 2×10¹¹ PFU, or a dose described in Section 6. In some embodiments, a priming composition comprises about 10 μg to about 1000 μg one or more antigenic proteins. In certain embodiments, a priming composition comprises about 10 μg to about 1000 μg one or more nucleic acid sequences encoding one or more antigenic proteins.

In certain embodiments, a priming composition further comprises an immune-potentiating compound such as cyclophosphamide (CPA).

### Boost Compositions

In one aspect, provided herein are boost compositions or compositions for a boost that may be used in the methods presented herein. In a specific embodiment, a boost composition is used to induce an immune response to one or more neoantigens in a subject. In certain embodiments, a boost composition is used to induce an immune response to 2 to about 20 neoantigens. In some embodiments, a boost composition is used to induce an immune response to 2, 3, 4, 5, 6, 7, 8, 9, or 10 neoantigens in a subject. In certain embodiments, a boost composition is used to induce an immune response to 1 to 3, 1 to 5, 2 to 4, 2 to 5, 2 to 6, 2 to 8, 5 to 8, 5 to 10, or 8 to 10 neoantigens in a subject. Any combination of the stated upper and lower limits is also envisaged. In a specific embodiment, a boost composition is one described in Section 6, infra, or similar compositions as described in Section 6, infra with different neoantigens

Generally, the methods presented herein utilize one or more boosts that comprise an oncolytic virus. Without wishing to be bound by theory or mechanism, an oncolytic virus may act as an adjuvant in a boost composition. By “oncolytic virus” is meant any one of a number of viruses that have been shown, when active, to specifically replicate and kill tumour cells in vitro or in vivo. These viruses may naturally be oncolytic viruses, or the viruses may have been modified to produce or improve oncolytic activity. In certain embodiments the term may encompass attenuated, replication defective, inactivated, engineered, or otherwise modified forms of an oncolytic virus suited to purpose.

In certain aspects, the methods presented herein utilize boosts that comprise a virus that is replication-competent and exhibits local replication in a subject, that is, replicates in only a subset of cell types in the subject, wherein the replication does not put the subject at risk. For example, the virus may replicate in immune organs (e.g., one or more lymph nodes, spleen or both), tumour cells, or both immune organs and tumor cells. While for ease of description, the methods and boost compositions presented herein generally refer to oncolytic viruses, it is understood that such methods and compositions can utilize and comprise such a virus.

In one embodiment, the oncolytic virus is attenuated. For example, in certain embodiments, the oncolytic virus may have reduced virulence, but still be viable or “live.” In one embodiment, the oncolytic virus exhibits reduced virulence relative to wild-type virus, but is still replication-competent. In one embodiment, the oncolytic virus is replication defective. In one embodiment, the oncolytic virus is inactivated (e.g., is UV inactivated).

In one embodiment, an oncolytic virus is a Rhabdovirus. “Rhabdovirus” include, inter alia, one or more of the following viruses or variants thereof: Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Maraba virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus. In certain aspects, a Rhabdovirus may refer to the supergroup of Dimarhabdovirus (defined as rhabdovirus capable of infecting both insect and mammalian cells).

In a particular embodiment, the Rhabdovirus is a Farmington virus or an engineered variant thereof. For exemplary, non-limiting examples of nucleotide sequences of the Farmington virus genome see GenBank Accession Nos. KC602379.1 (Farmington virus strain CT114); and HM627182.1. As is well-known, rhabdoviruses are negative-strand RNA viruses. As such, it is understood that nucleotide sequences of their genomes can include RNA and reverse complement versions of these representative nucleotide sequences.

In another particular embodiment, the Rhabdovirus is a Maraba virus or an engineered variant thereof. In one embodiment, for example, the oncolytic virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396 and U.S Patent Application Publication No. US2015/0275185, which are incorporated herein by reference, wherein it is referred to as SEQ ID NO: 4. An example of the Maraba G protein is described in PCT Application No. PCT/IB2010/003396 and U.S Patent Application Publication No. US2015/0275185, wherein it is referred to as SEQ ID NO: 5. In one embodiment, the oncolytic virus is the Maraba double mutant (“Maraba DM”) described in PCT Application No. PCT/IB2010/003396 and U.S Patent Application Publication No. US2015/0275185. In one embodiment, the oncolytic virus is the “Maraba MG1” described in PCT Application No. PCT/CA2014/050118; U.S. patent Ser. No. 10/363,293; and U.S Patent Application Publication No. US2019/0240301, which are incorporated herein by reference. As used herein, Maraba MG1 may be referred to as “MG1 virus.”

In another particular embodiment, the Rhabdovirus is a Farmington virus or an engineered variant thereof. In one embodiment, the oncolytic virus is a Farmington virus described in International Patent Application No. PCT/CA2012/050385, U.S. Patent Application Publication No. US2016/028796514 and International Patent Application No. PCT/CA2019/050433.

In one embodiment, the oncolytic virus is a vaccinia virus, measles virus, or a vesicular stomatitis virus.

In certain embodiments, the oncolytic virus is a vaccinia virus, e.g., a Copenhagen (see, e.g., GenBank M35027.1), Western Reserve, Wyeth, Lister (se, e.g., GenBank KX061501.1; DQ121394.1), EM63, ACAM2000, LC16m8, CV-1, modified vaccinia Ankara (MV A), Dairen I, GLV-1h68, IE1D-J, L-IVP, LC16m8, LC16mO, Tashkent, Tian Tan (see, e.g., AF095689.1), or WAU86/88-1 virus (for representative, non-limiting examples of nucleotide sequences, see the GenBank Accession Nos. provided in parentheses). In one embodiment, the vaccinia virus is a vaccinia virus with one or more beneficial mutations and/or one or more gene deletions or gene inactivations. For example, in certain embodiments, the vaccinia virus is a CopMD5p, CopMD3p, or CopMD5p3p vaccinia virus as described in WO 2019/134049, which is incorporated herein by reference in its entirety, and in particular for its description of these vaccinia viruses. In a specific embodiment, the vaccinia virus is CopMD5p3p vaccinia virus with a B8R deletion as described in WO 2019/134049.

In one embodiment, the virus is an oncolytic adenovirus, e.g., an adenovirus comprising a deletion in E1 and E3, which renders the adenovirus susceptible to p53 inactivation. Because many tumours lack p53, such a modification effectively renders the virus tumour-specific, and hence oncolytic. In one embodiment, the adenovirus is of serotype 5.

In one embodiment, a boost comprises an oncolytic virus that comprises a genome comprising a transgene, wherein the transgene encodes and expresses a protein in a subject, wherein the protein or a fragment thereof is capable of inducing an immune response to at least one neoantigen, and wherein the oncolytic virus is immunologically distinct from an oncolytic virus used in a subsequent boost of a method presented herein. In some embodiments, the oncolytic virus is immunologically distinct from an oncolytic virus used in a boost and a subsequent boost of a method presented herein.

In certain embodiments, an oncolytic virus is immunologically distinct from the oncolytic virus utilized in at least the first post-prime boost in a heterologous method described herein. In some embodiments, an oncolytic virus is immunologically distinct from the oncolytic viruses utilized in each of the boosts in a heterologous boost method described herein.

In another embodiment, a boost comprises an oncolytic virus and a peptide, wherein the peptide or fragment thereof is capable of inducing an immune response to at least one neoantigen, that is an antigenic protein, and wherein the oncolyic virus is immunologically distinct from an oncolytic virus used in at least the immediately subsequent boost. The oncolytic virus and peptide may be formulated in one composition or different compositions. A composition the oncolytic virus and a composition comprising the peptide may be formulated for the same route or different routes of administration to a subject. In some embodiments, the oncolytic virus is immunologically distinct from an oncolytic virus used in each of the boosts of a method presented herein. In certain embodiments, the oncolytic virus comprises a genome that comprises a transgene or a nucleic acid sequence that expresses an antigenic protein.

In another embodiment, a boost comprises a first composition and a second composition, wherein the first composition comprises an oncolytic virus, and the second composition comprises a peptide, wherein the peptide or fragment thereof is capable of inducing an immune response to at least one neoantigen, that is an antigenic protein, and wherein the oncolyic virus is immunologically distinct from an oncolytic virus used in at least the immediately subsequent boost. The first composition and second composition may be formulated for the same or a different route of administration to a subject. In some embodiments, the oncolytic virus is immunologically distinct from an oncolytic virus used in each of the boosts of a method presented herein. In certain embodiments, the oncolytic virus comprises a genome that comprises a transgene or a nucleic acid sequence that expresses an antigenic protein.

In some embodiments, a boost may comprise (i) one or more peptides capable of inducing an immune response to the one or more neoantigens of interest, that is, may comprise one or more antigenic proteins, and (ii) an oncolytic virus that comprises a genome comprising a transgene(s) or nucleic acid sequence(s), wherein the transgene(s) or nucleic acid sequence(s) express one or more proteins capable of inducing an immune response to the one or more neoantigens of interest, that is, express one or more antigenic proteins. In particular embodiments, a boost comprises one or more peptides capable of inducing an immune response to a first subset of the neoantigens of interest, and an oncolytic virus that comprises a genome comprising a transgene(s) or nucleic acid sequence(s), wherein the transgene(s) or nucleic acid sequence(s) express one or more proteins capable of inducing an immune response to a second subset of the neoantigens of interest. In certain embodiments, the first subset and the second subset of neoantigens of interest do not overlap. In other embodiments, the first subset and the second subset of neoantigens of interest are overlapping subsets. In certain embodiments, the first subset of neoantigens are public neoantigens and the second subset are private neoantigens. In some embodiments, the first and second subsets are private or public neoantigens, or vice versa. In certain embodiments, a boost comprises (i) one or more peptides capable of inducing an immune response to the neoantigens of interest, and (ii) an oncolytic virus comprises a genome that comprises transgene(s) or nucleic acid sequence(s), wherein the transgene(s) or nucleic acid sequence(s) expresses one or more proteins capable of inducing an immune response to the neoantigens of interest. In some embodiments, the one or more peptides and the oncolytic virus are administered in the same composition. In other embodiments, the one or more peptides and the oncolytic virus are administered in different compositions. The different compositions may be formulated for administration by the same or a different route of administration.

In some embodiments, a boost may comprise (i) one or more peptides capable of inducing an immune response to the one or more neoantigens of interest, that is, may comprise one or more antigenic proteins, and (ii) an oncolytic virus that does not comprise a genome that comprises a nucleic acid sequence or transgene that expresses an antigenic protein. A virus that does not comprise a genome that comprises nucleic acid sequence or transgene that expresses the antigenic protein refers to a virus that does not produce the antigenic protein and does not cause a cell infected by the virus to produce the protein. For example, the oncolytic virus may lack a nucleic acid sequence that encodes the amino acid sequence of the antigenic protein, or lack nucleic acid sequences necessary for the transcription and/or translation required for the virus to express the antigenic protein or to cause a cell infected by the virus to express the antigenic protein. In another example, the oncolytic virus may lack a nucleic acid sequence that encodes the amino acid sequence of the antigenic protein, and lack nucleic acid sequences necessary for the transcription and/or translation required for the virus to express the antigenic protein or to cause a cell infected by the virus to express the antigenic protein.

In a particular embodiment, an oncolytic virus is not engineered to (i) contain a transgene or nucleic acid sequence that encodes the amino acid sequence of the antigenic protein, or (ii) contain nucleic acid sequences necessary for the transcription and/or translation required for the virus to express the antigenic protein or to cause a cell infected by the virus to express the antigenic protein. In another embodiment, an oncolytic virus is not engineered to (i) contain a transgene or nucleic acid sequence that encodes the amino acid sequence of the antigenic protein, and (ii) contain nucleic acid sequences necessary for the transcription and/or translation required for the virus to express the antigenic protein or to cause a cell infected by the virus to express the antigenic protein.

In certain embodiments, an oncolytic virus that does not comprise a transgene or nucleic acid sequence that expresses the antigenic protein, the antigenic protein is not physically associated with and/or connected to the virus. For example, in certain embodiments, the antigenic protein (i) is not attached to, conjugated to or otherwise covalent bonded to the oncolytic virus, (ii) does not become attached to, conjugated to or otherwise covalently bonded to the oncolytic virus, (iii) does not non-covalently interact with the oncolytic virus, or (iv) does not form non-covalent interactions with the oncolytic virus. In some embodiments, two, three or all of the following apply to the antigenic protein: (i) the antigenic protein is not attached to, conjugated to or otherwise covalent bonded to the oncolytic virus, (ii) the antigenic protein does not become attached to, conjugated to or otherwise covalently bonded to the oncolytic virus, (iii) the antigenic protein does not non-covalently interact with the oncolytic virus, and (iv) the antigenic protein does not form non-covalent interactions with the oncolytic virus. In other particular embodiments, the antigenic protein is may be physically associated with and/or connected to the virus. For example, in particular embodiments, the antigenic protein (i) may be attached to, conjugated to or otherwise covalent bonded to the virus, (ii) may become attached to, conjugated to or otherwise covalently bonded to the virus, (iii) may non-covalently interact with the virus, or (iv) form non-covalent interactions with the virus. In some embodiments, one, two, three or all of the following apply to the antigenic protein (i) may be attached to, conjugated to or otherwise covalent bonded to the virus, (ii) may become attached to, conjugated to or otherwise covalently bonded to the virus, (iii) may non-covalently interact with the virus, and (iv) form non-covalent interactions with the virus.

In certain embodiments, a boost comprises one or more antigenic proteins. In some embodiments, a boost comprises one or more antigenic proteins, wherein at least one antigenic protein ranges in length from about 8 to about 500 amino acids. For example, at least one antigenic protein may be at least about 8, at least about 10, at least about 20, at least about 25, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, one or more of the antigenic proteins of a boost may be synthetic proteins. In some embodiments, one or more antigenic proteins of a boost may be recombinant proteins.

In certain embodiments, a boost comprises an antigenic protein, wherein the antigenic protein may comprise the entire amino acid sequence of the neoantigen of interest. In such embodiments, the antigenic protein may be as long or longer than the neoantigen of interest. In some embodiments, a boost comprises an antigenic protein, wherein antigenic protein may comprise an amino acid sequence shorter than the neoantigen of interest, but a minimum of about 8 amino acid residues, about 9 amino acid residues, about 10 amino acid residues, about 11 amino acid residues, or about 12 amino acid residues in length.

In certain embodiments in which a boost comprises an oncolytic virus that comprises a genome comprising a transgene, the transgene comprises a nucleic acid sequence that encodes an antigenic protein such that it is expressed in the subject. The transgene may also include additional sequences, such as, e.g., viral regulatory signals (e.g., gene end, intergenic, and/or gene start sequences) and Kozak sequences. Generally, the total length of a transgene is limited only by the nucleic acid carrying capacity of the particular virus, that is, the amount of nucleic acid that can be inserted into the genome of the virus without preventing a sufficient amount of the protein encoded by the transgene to be produced. In specific embodiments, a sufficient amount of the protein encoded by the transgene is enough to induce an immune response to a neoantigen. In certain embodiments, the total length of a transgene is limited only by the nucleic acid carrying capacity of the particular virus, that is, the amount of nucleic acid that can be inserted into the genome of the virus without significantly inhibiting the pre-insertion replication capability of the virus. In some embodiments, the amount of nucleic acid inserted into the genome of a virus does not significantly inhibit the pre-insertion replication capability of the virus if it does not reduce the replication by more than about 0.5 log, about 1 log, about 1.5 log, about 2 logs, about 2.5 logs, or about 3 logs in a particular cell line relative the replication of the virus absent the insert in the same cell line. In particular embodiments, for example, in instances where the virus is a Farmington virus or a Maraba virus, for example an MG1 virus, a transgene of about 3-5 kb, e.g., about 3 kb, about 3.5 kb, about 4 kb, about 4.5 kb, or about 5 kb, may be inserted into the virus genome. In the case of Maraba virus, e.g., MG1 virus, the nucleic acids expressing the antigenic proteins may, for example, be inserted into the Maraba genome between the G and L gene sequences. In the case of Farmington virus, e.g., FMT virus, the nucleic acids expressing the antigenic proteins may, for example, be inserted into the Farmington genome between the N and P gene sequences. Techniques known in the art may be used to insert a transgene into the genome of a virus and to assess the presence of the inserted transgene in the genome.

In certain embodiments where a boost comprises an oncolytic virus that comprises a genome comprising a transgene, wherein the transgene that encodes and expresses one or more antigenic proteins in a subject, at least one antigenic protein may range in length from about 8 to about 500 amino acids. In particular embodiments, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters.

In instances where a transgene encodes and expresses one or more antigenic proteins in a subject, in certain embodiments, the transgene can express the more than one antigenic protein as a single, longer protein. In instances wherein two or more antigenic proteins are expressed as part of a single, longer protein, in certain embodiments, the portion(s) of the longer protein corresponding to at least one individual antigenic protein fall(s) within these length parameters. In other embodiments, the portions of the longer protein corresponding to each of the individual antigenic proteins fall within these length parameters.

In certain embodiments where a transgene encodes and expresses an antigenic protein in a subject, the antigenic protein may comprise the entire amino acid sequence of the neoantigen of interest. In such embodiments, the antigenic protein may be as long or longer than the neoantigen of interest.

In some embodiments, an oncolytic virus comprises a genome that comprises transgene or nucleic acid sequences, wherein the transgene or nucleic acid sequences express x number of antigenic proteins, the virus may comprise a nucleic acid for each of the antigenic proteins, that is, a first nucleic acid that expresses the first antigenic protein, a second nucleic acid that expresses the second antigenic protein, etc., up to and including an xth nucleic acid that encodes the xth antigenic protein. In particular embodiments, the first antigenic protein is capable of inducing an immune response to a first neoantigen, the second antigenic protein is capable of inducing an immune response to a second neoantigen, etc., up to and including the xth antigenic protein being capable of inducing an immune response to an xth neoantigen. In certain embodiments, the transgene or nucleic acid sequences that express x number of antigenic proteins does not prevent a sufficient amount of the protein encoded by the transgene to be produced. In specific embodiments, a sufficient amount of the protein encoded by the transgene is enough to induce an immune response to the xth neoantigen. In a specific embodiment, the transgene or nucleic acid sequences that express x number of antigenic proteins does not significantly inhibit the pre-insertion replication capability of the virus if the transgene or nucleic acid sequence inserted into the viral genome does not reduce the replication of the virus by more than about 0.5 log, about 1 log, about 1.5 log, about 2 logs, about 2.5 logs, or about 3 logs in a particular cell line relative the replication of the virus absent the insert in the same cell line.

Within the virus, a nucleic acid sequence that expresses a particular antigenic protein may be contiguous to or separate from a nucleic acid sequence that expresses a different antigenic protein. In certain embodiments, each of the nucleic acid sequences expressing the antigenic protein may be present in the virus as a transgene. In some embodiments, each of the nucleic acid sequences expressing antigenic proteins is a fusion protein. As noted above, generally, the total length or lengths of such nucleic acid or nucleic acid sequences within the virus need only be limited by the nucleic acid carrying capacity of the virus. In certain embodiments, the nucleic acid sequences may express antigenic proteins as individual proteins. In certain embodiments, nucleic acid sequences may express antigenic proteins together as part of a longer protein. In certain embodiments, nucleic acid sequences may express certain of antigenic proteins as individual proteins and certain of antigenic proteins together as part of a longer protein. In instances where two or more antigenic proteins are expressed as part of a longer protein, the antigenic proteins may be adjacent to each other, with no intervening amino acids between them, or may be separated by an amino acid spacer. In certain embodiments involving a longer protein, some of antigenic proteins may be adjacent to each other and others may be separated by an amino acid spacer. In certain embodiments, the longer protein comprises one or more cleavage sites, for example, one or more proteasomal cleavage sites. In particular embodiments, the protein comprises one or more amino acid spacers that comprise one or more cleavage sites, for example, one or more proteasomal cleavage sites. See, e.g., Section 6, infra, for examples of nucleic acid sequences encoding one or more antigenic proteins.

In certain embodiments, a boost described herein further comprises an adjuvant. In certain embodiments, the adjuvant can potentiate an immune response to an antigen or modulate it toward a desired immune response. In some embodiments, the adjuvant can potentiate an immune response to an antigen and modulate it toward a desired immune response. In one embodiment, the adjuvant is polyI:C.

In some embodiments, a boost described herein further comprises a liposome(s) or a nanoparticle(s). In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate an antigenic protein or an oncolytic virus, or both. See, e.g., Sahin et al. (2014), mRNA-based therapeutics developing a new class of drugs. NATURE REVIEWS DRUG DISCOVERY, 13(10):759-780; Su et al. (2011) In vitro and in vivo mRNA delivery using lipid-enveloped pH-responsive polymer nanoparticles, MOLECULAR PHARMACEUTICALS, 8 (3):-774-778; Phua et al., (2014) Messenger RNA (mRNA) nanoparticle tumour vaccination, NANOSCALE, 6(14):7715-7729; Bockzkowski et al., Dendritic cells pulsed with RNA are potent antigen presenting cells in vitro and in vivo, JOURNAL OF EXPERIMENTAL MEDICINE, 184(2):465-472.

In certain embodiments, a boost described herein does not comprise a liposome(s) or a nanoparticle(s). In some embodiments, an antigenic protein or an oncolytic virus is not encapsulated in a delivery vehicle such as a liposomal preparation or nanoparticle. In a specific embodiment, an antigenic protein is not encapsulated in a delivery vehicle such as a liposomal preparation or nanoparticle. In another embodiment, an oncolytic virus and an antigenic protein are not encapsulated in a delivery vehicle such as a liposomal preparation or nanoparticle.

In some embodiments, a boost described herein further comprises a liposome(s) or a nanoparticle and an adjuvant. In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate (1) an antigenic protein or an oncolytic virus and (2) an adjuvant. In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate (1) an antigenic protein, (2) an oncolytic virus and (3) an adjuvant.

In one embodiment, a boost is formulated for intravenous, intramuscular, subcutaneous, intraperitoneal or intratumoral administration. When a boost is to be administered in parts, different parts of the boost may be formulated for the same or different routes of administration. For example, when a boost comprises a first composition and a second composition, wherein the first composition comprises an oncolytic virus, and the second composition comprises an antigenic protein, the first composition may be administered by the same or a different route than the second composition. In a particular embodiment, a boost is formulated for intravenous administration. In another embodiment, a boost is formulated for subcutaneous or intramuscular administration.

In certain embodiments, a boosting composition comprises 1×10⁷ to 5×10¹² PFU of an oncolytic virus. For example, in some embodiments, a boosting composition comprises 1×10⁷ to 1×10¹² PFU of an oncolytic virus. In certain embodiments, a boosting composition comprises about 1×10¹¹ PFU, about 2×10¹¹ PFU, or a dose described in Section 6. In some embodiments, a boosting composition comprises about 10 μg to about 1000 μg one or more antigenic proteins.

In certain embodiments, a boost further comprises an immune-potentiating compound such as cyclophosphamide (CPA).

### Methods of Inducing an Immune Response to Neoantigens

In one aspect, provided herein are methods for inducing an immune response to one or more neoantigens in a subject, comprising administering a dose of a priming composition and subsequently administering at least one boost. In a specific embodiment, provided herein are methods of inducing an immune response to one or more neoantigens in a subject, comprising administering a prime and one or more boosts. For example, in certain embodiments, such methods induce an immune response to 2 to about 20 neoantigens, e.g., 2 to about 10 neoantigens, 2-5 neoantigens, for example 2, 3, 4 or 5 neoantigens. The priming composition may be one described in Section 5.2 or 6. The boost may comprise at least one boosting composition described in Section 5.3 or 6. In some embodiments, the methods involve administering multiple doses of a priming composition. In certain embodiments, the methods involve administering two sequential heterologous boosts. For example, the methods involve administering a priming composition described in Section 5.2 and two boosting compositions described in Section 5.3.

The term “subject,” as used herein, refers to a mammal, for example, a non-human mammal, a primate, e.g., a non-human primate, or a human. In one embodiment, a subject is a human subject. In certain embodiments, a subject has a pre-existing immunity to a neoantigen of interest. In certain embodiments, a subject is naïve with respect to immunity to a neoantigen of interest. In specific embodiments, a subject has cancer or has been diagnosed as having cancer.

In another aspect, provided herein are sequential heterologous boost methods designed to induce an immune response to one or more neoantigens of interest. For example, in certain embodiments, such sequential heterologous boost methods induce an immune response to 2 to about 20 neoantigens, e.g., 2 to about 10 neoantigens, 2-5 neoantigens, for example 2, 3, 4 or 5 neoantigens. The sequential heterologous boost methods presented herein utilize oncolytic virus-comprising boosts wherein any two consecutive boosts utilize oncolytic viruses that are immunologically distinct from each other. Boosts that utilize oncolytic viruses that are immunologically distinct from each other may be referred to herein as heterologous boosts. The sequential heterologous boost methods presented herein may, for example, utilize any of the antigenic proteins, priming compositions and/or boost compositions described herein.

In certain embodiments, a sequential heterologous boost method as presented herein is a method of inducing an immune response to one or more neoantigens of interest in a subject, wherein the subject has a pre-existing immunity to the one or more neoantigens of interest. In certain embodiments, a sequential heterologous boost method as presented herein is a method of inducing an immune response to one or more neoantigens of interest in a subject, wherein the subject is naïve with respect to immunity to the one or more neoantigens of interest.

In particular embodiments, a sequential heterologous boost method as presented herein is a method of inducing an immune response to one or more neoantigens of interest in a subject, wherein the subject has been identified as having a pre-existing immunity to the one or more neoantigens of interest, and wherein the method comprises administering to the subject at least one consecutive heterologous boost, such that an immune reaction to the one or more neoantigens of interest. In certain embodiments, the method comprises administering to the subject a dose of a priming composition prior to boosting.

In other particular embodiments, a sequential heterologous boost method as presented herein is a method of inducing an immune response to one or more neoantigens in a subject, wherein the method comprises determining whether a subject has a pre-existing immunity to the one or more neoantigens of interest, and subsequently administering to the subject at least one sequential heterologous boost, such that an immune response to the one or more neoantigens is induced. For example, determining whether a subject has a pre-existing immunity to the one or more neoantigens of interest may comprise determining whether the subject contains CD8+ T cells specific for the one or more neoantigens of interest, e.g., determining whether peripheral blood from the subject contains antigen-specific interferon gamma positive CD8+ T cells. In embodiments where a subject is determined to have a preexisting immunity, the method further comprises administering to the subject at least one consecutive heterologous boost, such that an immune reaction to the one or more neoantigens of interest is induced, and may, in certain embodiments, comprise administering to the subject a dose of a priming composition prior to boosting.

In certain embodiments, a sequential heterologous boost method as presented herein is a method of inducing an immune response to one or more neoantigens of interest in a subject, wherein the subject is naïve with respect to immunity to the one or more neoantigens of interest. In certain embodiments, a sequential heterologous boost method as presented herein is a method of inducing an immune response to one or more neoantigens of interest, in a subject, wherein the subject is one that has been identified as naïve with respect to immunity to the one or more neoantigens of interest, and wherein the method comprises administering to the subject a dose of a priming composition and, subsequently, at least one pair of consecutive heterologous boosts such that an immune response to the neoantigen or neoantigens is induced.

In certain embodiments, a sequential heterologous boost method as presented herein is a method of inducing an immune response to one or more neoantigens of interest in a subject, wherein the method comprises determining whether a subject is naïve with respect to immunity to the one or more neoantigens of interest, and subsequently administering to the subject a dose of a priming composition that induces an immune response to the neoantigen or neoantigens, and subsequent to the administration of the priming composition, administering to the subject at least one pair of consecutive heterologous boosts such that an immune response to the neoantigen or neoantigens is induced. For example, determining whether a subject is naïve with respect to immunity to the one or more neoantigens of interest may comprise determining whether the subject contains CD8+ T cells specific for the one or more neoantigens of interest, e.g., determining whether peripheral blood from the subject contains antigen-specific interferon gamma positive CD8+ T cells.

With respect to inducing an immune response to at least one neoantigen, it will be appreciated that the at least one protein of the priming composition (or the protein(s) expressed by a nucleic acid of a priming virus contained in the priming composition, as appropriate) and the at least one protein of the boost(s) (or the protein(s) expressed by a nucleic acid(s) of the oncolytic viruses of boost(s), as appropriate) need not be exactly the same in order to accomplish this. Likewise, it will be appreciated that the at least one protein of any of the boosts (or the protein(s) expressed by a nucleic acid(s) of the oncolytic viruses of any of the boost(s), as appropriate) need not be exactly the same in order to accomplish this. For example, the proteins may comprise sequences that partially overlap, with the overlapping segment(s) comprising a sequence corresponding to a sequence of the neoantigen, or a sequence designed to induce an immune reaction to the neoantigen, thereby allowing an effective prime and boosts to the neoantigen to be achieved. For instance, the proteins may comprise sequences that partially overlap, with the overlapping segment(s) comprising a sequence corresponding to a sequence of the neoantigen, or a sequence designed to induce an immune reaction to the neoantigen, thereby allowing an effective prime and boosts to the neoantigen to be achieved. For example, the proteins may both share a sequence that comprises at least one epitope of the neoantigen. In another example, the proteins may comprise sequences that partially overlap, with the overlapping segment(s) comprising a sequence corresponding to the sequence of the neoantigen.

For a particular neoantigen, for example, in one embodiment the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are at least about 70% identical, at least about 80% identical, at least about 90% identical, at least about 95% identical, or are identical. In another embodiment, the sequence of the protein of the priming composition (or the protein expressed by a nucleic acid sequence, or the protein expressed by a nucleic acid of a virus contained in the priming composition) and the sequence of the protein of each of the boosts (or the protein expressed by a nucleic acid of an oncolytic virus of each of the boosts) are at least about 70% identical, at least about 80% identical, at least about 90% identical, at least about 95% identical, or are identical.

For a particular neoantigen, in one embodiment, for example, the sequence of the protein of each of the boosts (or the protein expressed by a nucleic acid of an oncolytic virus of each of the boosts) are identical. In another such embodiment, for example, the sequence of the protein of the priming composition (or the protein expressed by a nucleic acid sequence, or the protein expressed by a nucleic acid of a virus contained in the priming composition), and the sequence of the protein of each of the boosts (or the protein expressed by a nucleic acid of an oncolytic virus of each of the boosts) are identical.

In additional embodiments, for a particular neoantigen, the sequence of the protein of the priming composition (or the protein expressed by a nucleic acid sequence, or the protein expressed by a nucleic acid of a priming virus contained in the priming composition composition) and the sequence of the protein of each of the boosts (or the protein expressed by a nucleic acid of an oncolytic virus of each of the boosts) are at least about 70% identical, at least about 80% identical, at least about 90% identical, at least about 95% identical, or are identical, and the sequence of the protein of each of the boosts (or the protein expressed by a nucleic acid of an oncolytic virus of each of the boosts) are at least about 70% identical, at least about 80% identical, at least about 90% identical, at least about 95% identical, or are identical to each other. In another embodiment, the sequence of the protein of the priming composition (or the protein expressed by a nucleic acid sequence, or the protein expressed by a nucleic acid of a priming virus contained in the priming composition) and the sequence of the protein of each of the boosts (or the protein expressed by a nucleic acid of an oncolytic virus of each of the boosts) are at least about 70% identical, at least about 80% identical, at least about 90% identical, at least about 95% identical, or are identical, and the sequence of the protein of any of the boosts (or the protein expressed by a nucleic acid of an oncolytic virus of any of the boosts) are at least about 70% identical, at least about 80% identical, at least about 90% identical, at least about 95% identical, or are identical to each other.

In further embodiments, for a particular neoantigen, the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are at least about 70% identical, at least about 80% identical, at least about 90% identical, at least about 95% identical, or are identical, and the sequence of the protein of each of the boosts (or the protein expressed by a nucleic acid of an oncolytic virus of each of the boosts) are at least about 70% identical, at least about 80% identical, at least about 90% identical, at least about 95% identical, or are identical to each other. In another embodiment, the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are at least about 70% identical, at least about 80% identical, at least about 90% identical, at least about 95% identical, or are identical, and the sequence of the protein of any of the boosts (or the protein expressed by a nucleic acid of an oncolytic virus of any of the boosts) are at least about 70% identical, at least about 80% identical, at least about 90% identical, at least about 95% identical, or are identical to each other.

In specific embodiments, for a particular neoantigen, for example, in one embodiment the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are identical over a contiguous stretch of about 70%, about 80%, about 90% or 95% of either protein. In another embodiment, the sequence of the protein of the priming composition (or the protein expressed by a nucleic acid sequence, or the protein expressed by a nucleic acid of a priming virus contained in the priming composition) and the sequence of the protein of each of the boosts (or the protein expressed by a nucleic acid of an oncolytic virus of each of the boosts) are identical over a contiguous stretch of about 70%, about 80%, about 90% or 95% of either protein.

In additional specific embodiments, for a particular neoantigen, the sequence of the protein of the priming composition (or the protein expressed by a nucleic acid sequence, or the protein expressed by a nucleic acid of a priming virus contained in the priming composition) and the sequence of the protein of each of the boosts (or the protein expressed by a nucleic acid of an oncolytic virus of each of the boosts) are identical over a contiguous stretch of about 70%, about 80%, about 90% or 95% of either protein, and the sequence of the protein of each of the boosts (or the protein expressed by a nucleic acid of an oncolytic virus of each of the boosts) are identical over a contiguous stretch of about 70%, about 80%, about 90% or 95% of each other. In another embodiment, the sequence of the protein of the priming composition (or the protein expressed by a nucleic acid sequence, or the protein expressed by a nucleic acid of a priming virus contained in the priming composition) and the sequence of the protein of each of the boosts (or the protein expressed by a nucleic acid of an oncolytic virus of each of the boosts) are identical over a contiguous stretch of about 70%, about 80%, about 90% or 95% of either protein, and the sequence of the protein of any of the boosts (or the protein expressed by a nucleic acid of an oncolytic virus of any of the boosts) are identical over a contiguous stretch of about 70%, about 80%, about 90% or 95% of each other.

In further specific embodiments, for a particular neoantigen, the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are identical over a contiguous stretch of about 70%, about 80%, about 90% or 95% of either protein, and the sequence of the protein of each of the boosts (or the protein expressed by a nucleic acid of an oncolytic virus of each of the boosts) are identical over a contiguous stretch of about 70%, about 80%, about 90% or 95% of each other. In another embodiment, the sequence of the protein of the priming composition (or the protein expressed by a nucleic acid sequence, or the protein expressed by a nucleic acid of a priming virus contained in the priming composition) and the sequence of the protein of any of the boosts (or the protein expressed by a nucleic acid of an oncolytic virus of any of the boosts) are identical over a contiguous stretch of about 70%, about 80%, about 90% or 95% of either protein, and the sequence of the protein of any of the boosts (or the protein expressed by a nucleic acid of an oncolytic virus of any of the boosts) are identical over a contiguous stretch of about 70%, about 80%, about 90% or 95% of each other.

The population of at least two antigenic proteins from the prime and the population of at least two antigenic proteins from the boost may have complete, partial or no overlap in identity. In various embodiments, the at least two antigenic proteins of the prime and the boost are identical. In various embodiments, none of the at least two antigenic proteins of the prime and the boost are identical. In various embodiments, at least one of the at least two antigenic proteins from the first administration are identical to at least one of the at least two antigenic proteins from the second administration.

Utilization of one or more heterologous boosts may impart a substantially beneficial effect on the magnitude and/or duration of the resulting immune response, e.g., the CD8+ T cell response. The immune response may, for example, be measured by determining the absolute number of neoantigen-specific CD8+ T cells, for example, the number of antigen-specific interferon gamma (IFN-γ)-positive CD8+ T cells per ml of peripheral blood from the subject. See, e.g., Section 6, infra, and Pol et al. “Maraba virus as a potent oncolytic vaccine vector.” Molecular therapy: the journal of the American Society of Gene Therapy vol. 22, 2 (2014): 420-429. doi:10.1038/mt.2013.249 examples of methods for assessing the immune response induced by one or more heterologous boosts.

In certain embodiments of a sequential heterologous boost method presented herein, for a pair of consecutive heterologous boosts, e.g., the first and second consecutive heterologous boosts of the method, the peak immune response to a neoantigen of interest that is induced in a subject after administration of the second boost of the pair is equal to or higher than the peak immune response to the neoantigen induced by administration of the first boost in the pair. For example, in certain embodiments of a sequential heterologous boost method presented herein, for a pair of consecutive heterologous boosts, e.g., the first and second consecutive boosts of the method, the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log, about 0.75 log, about 1.0 log, about 1.2 log, about 1.5 log, or about 2.0 log higher than the peak immune response to the neoantigen induced by administration of first boost in the pair. The immune response may, for example, be measured by determining the absolute number of antigen-specific CD8+ T cells, for example, the number of antigen-specific interferon gamma (IFN-γ)-positive CD8+ T cells per ml of peripheral blood from the subject. See, e.g., Section 6, infra, for an example of a method for assessing the immune response induced by one or more heterologous boosts. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive heterologous boosts of the method, with respect to the immune response to a neoantigen of interest induced in a subject by administration of the second boost of the pair, for at least one week, two weeks, three weeks, four weeks, one month, two months or three months after administration of the second boost the immune response attained to the neoantigen remains equal to or higher than the peak immune response to the antigen induced with administration of first boost in the pair. The immune response may, for example, be measured by determining the percentage of neoantigen-specific CD8+ T cells (for example, the number of neoantigen-specific interferon gamma (IFN-γ)-positive CD8+ T cells) of total CD8+ T cells per ml of peripheral blood from the subject. See, e.g., Section 6, infra, for an example of a method for assessing the immune response induced by one or more heterologous boosts. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive heterologous boosts of the method, 1) the peak immune response to a neoantigen of interest that is induced in a subject after administration of the second boost of the pair is equal to or higher than the peak immune response to the neoantigen induced by administration of the first boost in the pair; and 2) with respect to the immune response to a neoantigen of interest induced in a subject by administration of the second boost of the pair, for at least one week, two weeks, three weeks, four weeks, one month, two months or three months after administration of the second boost the immune response attained to the neoantigen remains equal to or higher than the peak immune response to the antigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect may be observed with respect to the aggregate immune response to the neoantigens of interest.

In certain embodiments of a sequential heterologous boost method presented herein, for a pair of consecutive heterologous boosts, e.g., the first and second consecutive boosts of the method, 1) the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log, about 0.75 log, about 1.0 log, about 1.2 log, about 1.5 log, or about 2.0 log higher than the peak immune response to the neoantigen induced by administration of first boost in the pair; and 2) with respect to the immune response to a neoantigen of interest induced in a subject by administration of the second boost of the pair, for at least one week, two weeks, three weeks, 4 weeks, one month, two months or three months after administration of the second boost the immune response attained to the neoantigen remains equal to or higher than the peak immune response to the antigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, 1) the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log higher than the peak immune response to the antigen induced by administration of first boost in the pair; and 2) with respect to the immune response to a neoantigen of interest induced in a subject by administration of the second boost of the pair, for at least one month after administration of the second boost the immune response attained to the antigen remains equal to or higher than the peak immune response to the neoantigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, increase the immune response to each neoantigen of interest is increased following the second boost. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, the antigen-specific CD8+ T cells in peripheral blood following the latter boost comprise T effector cells (Teff cells) and T effector memory cells (Tem cells), and the majority of such cells do not exhibit an “exhausted” T cell phenotype. For example, in particular embodiments, less than about 15%, less than about 20%, less than about 30%, less than about 40% or less than about 50% of antigen-specific Teff cells and/or Tem cells are positive for PD-1, CTLA-4, and LAG-3. In other particular embodiments, less than about 15%, less than about 20%, less than about 30%, less than about 40% or less than about 50% of neoantigen-specific Teff cells and Tem cells are positive for PD-1, CTLA-4, and LAG-3. In yet other particular embodiments, less than about 15%, less than about 20%, less than about 30%, less than about 40% or less than about 50% of antigen-specific Teff cells and/or Tem cells are positive for PD-1, CTLA-4 or LAG-3. In still other particular embodiments, less than about 15%, less than about 20%, less than about 30%, less than about 40% or less than about 50% of antigen-specific Teff cells and Tem cells are positive for PD-1, CTLA-4, or LAG-3. In instances where the sequential heterologous boost method is a method of inducing an immune response to at least two antigens of interest in a subject, such an effect may be observed with respect to the immune response induced to least one of the neoantigens of interest. In other instances where the sequential heterologous boost method is a method of inducing an immune response to at least two antigens of interest in a subject, such an effect may be observed with respect to the aggregate immune response to the neoantigens of interest.

The sequential heterologous boost methods described herein utilize consecutive heterologous boosts, which are consecutive boosts wherein one of the boosts comprising a first oncolytic virus and the other boost comprising a second oncolytic virus that is immunologically distinct from the first oncolytic virus. In certain embodiments, the sequential heterologous boost methods described herein comprise two boosts, a first boost that comprises a first oncolytic virus, and a second, consecutive, heterologous boost comprising a second oncolytic virus that is immunologically distinct from the first oncolytic virus. In certain embodiments, the sequential heterologous boost methods described herein comprise more than two boosts, e.g., comprise 3, 4, 5 or more boosts, wherein any consecutive pair of boosts utilizes heterologous boosts.

For example, in certain embodiments, the sequential heterologous boost methods described herein comprise three boosts wherein the oncolytic virus of the first boost is immunologically distinct from the oncolytic virus of the second boost, and the oncolytic virus of the second boost is immunologically distinct from the oncolytic virus of the third boost. Such methods may comprise two or three oncolytic viruses, wherein the oncolytic viruses are distributed in the boosts in a manner that results in heterologous boost administration.

In another non-limiting example, the sequential heterologous boost methods described herein comprise four boosts wherein the oncolytic virus of the first boost is immunologically distinct from the oncolytic virus of the second boost, the oncolytic virus of the second boost is immunologically distinct from the oncolytic virus of the third boost, and the oncolytic virus of the third boost is immunologically distinct from the oncolytic virus of the fourth boost. Such methods may comprise two, three or four oncolytic viruses, wherein the oncolytic viruses are distributed in the boosts in a manner that results in heterologous boost administration.

In yet another non-limiting example, the sequential heterologous boost methods described herein comprise five boosts wherein the oncolytic virus of the first boost is immunologically distinct from the oncolytic virus of the second boost, the oncolytic virus of the second boost is immunologically distinct from the oncolytic virus of the third boost, the oncolytic virus of the third boost is immunologically distinct from the oncolytic virus of the fourth boost, and the oncolytic virus of the fourth boost is immunologically distinct from the oncolytic virus of the fifth boost. Such methods may comprise two, three, four or five oncolytic viruses, wherein the oncolytic viruses are distributed in the boosts in a manner that results in heterologous boost administration.

In one embodiment, a sequential heterologous boost method of inducing an immune response to a neoantigen in a subject comprises: (a) administering to the subject a dose of a priming composition that is capable of inducing an immune response to the neoantigen; (b) subsequently administering to the subject a dose of a first boost, wherein the first boost comprises a first oncolytic virus, wherein the first oncolytic virus comprises a genome that comprises a transgene or a nucleic acid sequence that encodes and expresses, in the subject, a protein that is capable of inducing an immune response to the neoantigen; and (c) subsequently administering to the subject a dose of a second, heterologous boost, wherein the heterologous boost comprises a second oncolytic virus, wherein the second oncolytic virus comprises a transgene or a nucleic acid sequence that encodes and expresses, in the subject, a protein that is capable of inducing an immune response to the neoantigen, and wherein the second oncolytic virus is immunologically distinct from the first oncolytic virus, such that an immune response to the neoantigen is induced in the subject.

In another embodiment, a sequential heterologous boost method of inducing an immune response to a neoantigen in a subject comprises: (a) administering to the subject a dose of a priming composition that is capable of inducing an immune response to the neoantigen; (b) subsequently administering to the subject a dose of a first boost, wherein the first boost comprises a first oncolytic virus, wherein the first oncolytic virus comprises a genome that comprises a transgene or a nucleic acid sequence that encodes and expresses, in the subject, a protein that is capable of inducing an immune response to the neoantigen; and (c) subsequently administering to the subject a dose of a second, heterologous boost, wherein the heterologous boost comprises a second oncolytic virus, wherein the second oncolytic virus comprises a transgene or a nucleic acid sequence that encodes and expresses, in the subject, a protein that is capable of inducing an immune response to the neoantigen, and wherein the second oncolytic virus is immunologically distinct from the first oncolytic virus; and (d) subsequently administering to the subject a dose of a third boost, wherein the third boost comprises an oncolytic virus that is immunologically distinct from the oncolytic virus of the second boost and that comprises a transgene or a nucleic acid sequence that expresses, in the subject, a protein that is capable of inducing an immune response to the neoantigen, such that an immune response to the neoantigen is induced in the subject. In particular embodiments, the oncolytic virus of the third boost is the first oncolytic virus, present in the first boost. In one non-limiting example, step (d) is performed at least about 60 days after step (b). In other non-limiting example, step (d) is performed at least about 120 days after step (b).

In certain embodiments, such a sequential heterologous boost method further comprises, subsequently to (d) a step (e) administering to the subject a dose of a fourth boost, wherein the fourth boost comprises an oncolytic virus that is immunologically distinct from the oncolytic virus of the third boost and that comprises a transgene or a nucleic acid sequence that encodes and expresses, in the subject, a protein that is capable of inducing an immune response to the neoantigen. In particular embodiments, the oncolytic virus of the fourth boost is the second oncolytic virus, present in the second boost. In one non-limiting example, step (e) is performed at least about 60 days after step (c). In other non-limiting example, step (e) is performed at least about 120 days after step (c).

In certain embodiments, such a sequential heterologous boost method further comprises, subsequently to (e) step (0 administering to the subject a dose of a fifth boost, wherein the fifth boost comprises an oncolytic virus that is immunologically distinct from the oncolytic virus of the fourth boost and that comprises a transgene or a nucleic acid sequence that encodes and expresses, in the subject, a protein that is capable of inducing an immune response to the neoantigen. In particular embodiments, the oncolytic virus of the fifth boost is the first oncolytic virus, present in the first boost. In other particular embodiments, the oncolytic virus of the fifth boost is the oncolytic virus present in the third boost. In one non-limiting example, step f) is performed at least about 60 days after step (d). In other non-limiting example, step (0 is performed at least about 120 days after step (d).

In certain aspects, the sequential heterologous boost methods presented herein are methods of inducing an immune response to one or more neoantigens of interest in a subject, wherein the boosts are heterologous boosts and at least one of the boosts comprises (a) one or more proteins capable of inducing an immune response to the neoantigen, that is, comprises one or more antigenic proteins, and (b) an oncolytic virus that does not comprise a transgene or a nucleic acid sequence that encodes and expresses, in the subject, the one or more antigenic proteins. In certain other aspects, the sequential heterologous boost methods presented herein are methods of inducing an immune response to one or more neoantigens of interest in a subject, wherein the boosts are heterologous boosts and at least one of the boosts comprises (a) one or more proteins capable of inducing an immune response to the one neoantigen(s) of interest, that is, comprises one or more antigenic proteins, and (b) an oncolytic virus that comprises a transgene or a nucleic acid sequence that encodes and expresses, in the subject, one or more proteins capable of inducing an immune response to the one or more neoantigen(s) of interest, that is, expresses one or more antigenic proteins.

In yet other aspects, the sequential heterologous boost methods presented herein are methods of inducing an immune response to one or more neoantigens of interest in a subject, wherein the boosts are heterologous boosts and 1) at least one of the boosts comprises a) one or more proteins capable of inducing an immune response to the one or more neoantigens, that is, comprises one or more antigenic proteins, and b) an oncolytic virus that does not comprise a transgene or a nucleic acid sequence that encodes and expresses, in the subject, the antigenic proteins; and 2) at least one of the boosts comprises a) one or more proteins capable of inducing an immune response to the one or more neoantigens of interest, that is, comprises one or more antigenic proteins, and b) an oncolytic virus that comprises a transgene or a nucleic acid sequence that encodes and expresses, in the subject, one or more proteins capable of inducing an immune response to the one or more neoantigens of interest, that is, expresses one or more antigenic proteins.

For example, in certain embodiments, a sequential heterologous boost method of inducing an immune response to a neoantigen in a subject presented herein, comprises a) administering to the subject a dose a priming composition; b) subsequently administering to the subject a dose of a first boost, wherein the first boost comprises a protein that is capable of inducing an immune response to the neoantigen, and a first oncolytic virus that does not comprise a transgene or a nucleic acid sequence that expresses the protein, wherein the protein and the first oncolytic virus are administered to the subject together or separately; and c) subsequently administering to the subject a dose of a second, heterologous boost, wherein the heterologous boost comprises a protein that is capable of inducing an immune response to the neoantigen, and a second oncolytic virus that does not comprise a transgene or a nucleic acid sequence that encodes and expresses the protein, wherein the protein and the second oncolytic virus are administered to the subject together or separately, and wherein the second oncolytic virus is immunologically distinct from the first oncolytic virus, such that an immune response to the neoantigen is induced in the subject. In particular embodiments, such sequential heterologous boost methods may comprise additional heterologous boosts, for example a third, fourth or fifth heterologous boost.

In one embodiment of the sequential heterologous boost methods described herein, at least one of the oncolytic viruses is a rhabdovirus. In a particular embodiment, the rhabdovirus is a Farmington virus. In another particular embodiment, the rhabdovirus is a Maraba virus, e.g., is an MG1 virus. In another embodiment, the first oncolytic virus and the second oncolytic virus are rhabdoviruses. In a particular embodiment, at least one of the rhabdoviruses is a Farmington virus. In another particular embodiment, at least one of the rhabdoviruses is a Maraba virus, e.g., is an MG1 virus. In yet another embodiment, one of the rhabdoviruses is a Farmington virus and one of the rhabdoviruses is a Maraba virus, e.g., an MG1 virus. In a specific embodiment, the first oncolytic virus is a Farmington virus and the second oncolytic virus is a Maraba virus, e.g., an MG1 virus. In another specific embodiment, the first oncolytic virus is a Maraba virus, e.g., an MG1 virus, and the second oncolytic virus is a Farmington virus.

In one embodiment of the sequential heterologous boost methods described herein, at least one of the oncolytic viruses is an adenovirus, a vaccinia virus, a measles virus, or a vesicular stomatitis virus. In another embodiment, the first and the second oncolytic virus are an adenovirus, a vaccinia virus, a measles virus, or a vesicular stomatitis virus. In a particular embodiment, either the first or the second oncolytic virus is a rhabdovirus and the other oncolytic virus is a vaccinia virus. In a specific embodiment, the first oncolytic virus is a rhabdovirus and the second oncolytic virus is a vaccinia virus. In another specific embodiment, first oncolytic virus is a vaccinia virus and the second oncolytic virus is a rhabdovirus. In a non-limiting example of such sequential heterologous boost methods, the rhabdovirus is a Farmington virus. In another such non-limiting example, the rhabdovirus is a Maraba virus, e.g., an MG-1 virus. In yet another such non-limiting example, the vaccinia virus is a CopMD5p, CopMD3p, or CopMD5p3p vaccinia virus. In yet another such non-limiting example, the vaccinia virus is CopMD5p3p with a B8R gene deletion.

In another embodiment, at least one of the oncolytic viruses is a rhabdovirus and at least one of the oncolytic viruses is a vaccinia virus, e.g., a CopMD5p, CopMD3p, or CopMD5p3p vaccinia virus. In another embodiment, at least one of the oncolytic viruses is a rhabdovirus and at least one of the oncolytic viruses is CopMD5p3p vaccinia virus with a B8R gene deletion. In another example of such sequential heterologous boost methods, the oncolytic viruses comprise at least one Farmington virus and at least one vaccinia virus, e.g., a CopMD5p, CopMD3p, or CopMD5p3p vaccinia virus. In another example of such sequential heterologous boost methods, the oncolytic viruses comprise at least one Farmington virus and at least CopMD5p3p vaccinia virus with a B8R gene deletion. In another example, the oncolytic viruses comprise at least one Maraba virus, e.g., an MG-1 virus and at least one vaccinia virus, e.g., a CopMD5p, CopMD3p, or CopMD5p3p vaccinia virus. In another example, the oncolytic viruses comprise at least one Maraba virus, e.g., an MG-1 virus and at least CopMD5p3p vaccinia virus with a B8R gene deletion. In yet another example the oncolytic viruses comprise at least one Farmington virus, at least one Maraba virus, e.g., an MG-1 virus, and at least one vaccinia virus, e.g., a CopMD5p, CopMD3p, or CopMD5p3p vaccinia virus. In yet another example the oncolytic viruses comprise at least one Farmington virus, at least one Maraba virus, e.g., an MG-1 virus, and at least CopMD5p3p vaccinia virus with a B8R gene deletion.

As used herein throughout, when two or more elements, may be administered together or separately, such elements may, e.g., be administered as a single composition or as part of more than one composition, and may be administered concurrently (whether as part of a single composition or as part of more than one composition), or sequentially.

In another embodiment, a sequential heterologous boost method of inducing an immune response to a plurality of neoantigens of interest in a subject comprises (a) administering to the subject a dose of a priming composition, wherein the priming composition induces an immune response to the plurality of neoantigens; (b) subsequently administering to the subject a dose of a first boost, wherein the first boost comprises a protein composition that is capable of inducing an immune response to the plurality of neoantigens of interest, and a first oncolytic virus that does not comprise a transgene or nucleic acid sequence that expresses, in the subject, a protein composition that is capable of inducing an immune response to any of the plurality of neoantigens of interest; and (c) subsequently administering to the subject a dose of a second, heterologous boost, wherein the heterologous boost comprises a protein composition that is capable of inducing an immune response to the plurality of neoantigens of interest, and a second oncolytic virus that does not comprise a transgene or nucleic acid sequence that expresses, in the subject, a protein composition that is capable of inducing an immune response to any of the plurality of neoantigens of interest, and wherein the second oncolytic virus is immunologically distinct from the first oncolytic virus, such that an immune response to plurality of neoantigens is induced in the subject. In particular embodiments, such sequential heterologous boost methods may comprise additional heterologous boosts, for example a third, fourth or fifth heterologous boost. In certain such embodiments, the protein composition in b) that is capable of inducing an immune response to the plurality of neoantigens of interest, and protein composition in c) that is capable of inducing an immune response to the plurality of neoantigens of interest may comprise one or more antigenic proteins. In particular embodiments, the protein composition in b) and the protein composition in c) are not identical. In certain such embodiments, a plurality of antigens of interest may be 2 to about 20 antigens, e.g., 2 to about 10 antigens, 2-5 antigens, for example 2, 3, 4 or 5 antigens.

In another embodiment, a sequential heterologous boost method of inducing an immune response to a plurality of neoantigens of interest in a subject comprises a) administering to the subject a dose of a priming composition, wherein the priming composition induces an immune response to the plurality of neoantigens; b) subsequently administering to the subject a dose of a first boost, wherein the first boost comprises a first protein composition that is capable of inducing an immune response to at least one of the plurality of neoantigens of interest, and a first oncolytic virus that comprises a genome that comprises one or more transgenes or nucleic acid sequences that express, in the subject, a second protein composition that is capable of inducing an immune response to at least one of the plurality of neoantigens of interest, such that, as a whole the first protein composition and the second protein composition are capable of inducing an immune response to the plurality of neoantigens of interest; and c) subsequently administering to the subject a dose of a second, heterologous boost, wherein the heterologous boost comprises a third protein composition that is capable of inducing an immune response to at least one of the plurality of neoantigens of interest, and a second oncolytic virus that comprises one or more transgenes or nucleic acid sequences that express, in the subject, a fourth protein composition that is capable of inducing an immune response to at least one of the plurality of neoantigens of interest such that, as a whole the first protein composition and the second protein composition are capable of inducing an immune response to the plurality of neoantigens of interest, and wherein the second oncolytic virus is immunologically distinct from the first oncolytic virus, such that an immune response to plurality of neoantigens is induced in the subject.

In particular embodiments, such sequential heterologous boost methods may comprise additional heterologous boosts, for example a third, fourth or fifth heterologous boost. In certain such embodiments, the first, second, third, and fourth protein composition may comprise one or more antigenic proteins. In particular embodiments, the first, second, third, and/or fourth protein compositions are not identical. In certain such embodiments, a plurality of antigens of interest may be 2 to about 20 antigens, e.g., 2 to about 10 antigens, 2-5 antigens, for example 2, 3, 4 or 5 antigens.

For example, in one embodiment, a sequential heterologous boost method of inducing an immune response to at least two antigens in a subject comprises a) administering to the subject a dose of a priming composition, wherein the priming composition induces an immune response to at least a first and a second neoantigen; b) subsequently administering to the subject a dose of a first boost, wherein the first boost comprises a first oncolytic virus that comprises a transgene or nucleic acid sequence that expresses, in the subject, a protein that is capable of inducing an immune response to at least the first neoantigen and a nucleic acid that expresses, in the subject, a protein that is capable of inducing an immune response to at least the second neoantigen; and c) subsequently administering to the subject a dose of a second, heterologous boost, wherein the heterologous boost comprises a second oncolytic virus comprises a genome that comprises a nucleic acid sequence that expresses, in the subject, a protein that is capable of inducing an immune response to at least the first neoantigen and a nucleic acid sequence that expresses, in the subject, a protein that is capable of inducing an immune response to at least the second neoantigen, and wherein the second oncolytic virus is immunologically distinct from the first oncolytic virus, such that an immune response to at least the first and the second neoantigens is induced in the subject. In particular embodiments, such sequential heterologous boost methods may comprise additional heterologous boosts, for example a third, fourth or fifth heterologous boost.

In certain embodiments of any of the sequential heterologous boost methods described herein, a dose of a priming composition that induces an immune response against greater than one antigen of interest may, for example, involve the administration of a single composition to a subject, or may involve the administration of more than one composition to the subject. For example, in instances where the priming composition is designed to induce an immune response to at least two neoantigens of interest, the prime dose may, in alternative embodiments, comprise a composition that comprise a composition that induces an immune response to at least the first and the second neoantigens, or, may comprise a first composition and a second composition, wherein the first composition induces an immune response to at least the first neoantigen, and the second composition induces an immune response to at least the second neoantigen. In embodiments where the prime dose comprises more than one composition, the compositions may be administered together or separately.

A dose e.g., a prime dose, a dose of a first boost, a dose of a second boost, a dose of a third boost and the like, as used herein, refers to an amount sufficient to achieve a recited or intended goal. In certain embodiments, a dose may be administered as a single composition. In other embodiments, a dose may be administered in parts. When administered in parts, e.g., 2, 3, or 4 parts, the parts may be administered concurrently or sequentially.

In certain embodiments of the sequential heterologous boost methods presented herein, the prime dose comprises a virus. In such embodiments, a prime dose may, for example, comprise about 1×10⁷ particle forming units (PFU) to about 5×10¹² PFU of virus. In certain embodiments, the prime dose comprises about 1×10¹¹ PFU, or 2×10¹¹ PFU of virus. In particular embodiments, the virus comprises a genome that comprises a transgene or a nucleic acid that expresses, in a subject, antigenic protein, as described herein. In other particular embodiments, the virus is a virus that does not comprise a nucleic acid that expresses the antigenic protein, as described herein. In certain embodiments, the virus is an adenovirus, for example, a serotype 5 adenovirus, e.g., a recombinant replication-incompetent human Adenovirus serotype 5.

In certain embodiments wherein a prime dose comprises one or more proteins capable of inducing an immune response to one or more neoantigens of interest, that is, comprises one or more antigenic proteins, the dose of such a prime may comprise about 10 μg to about 1000 μg of the one or more antigenic proteins. In particular embodiments, these amounts refer to the amount of antigenic protein present in a prime dose in the aggregate. In other particular embodiments, these amounts refer to the amount of each antigenic protein present in the prime dose.

In certain embodiments wherein a prime with a priming composition comprises an adoptive cell transfer of neoantigen-specific CD8+ T cells, such a prime may further comprise about 10 μg to about 1000 μg of the one or more antigenic proteins. In certain embodiments wherein a prime dose comprises an adoptive cell transfer of neoantigen-specific CD8+ T cells, such a prime may further comprise a virus that comprises a nucleic acid that expresses a protein capable of inducing an immune response to the antigen. In yet other embodiments wherein a prime with a priming composition comprises an adoptive cell transfer of neoantigen-specific CD8+ T cells, such a prime may further comprise about 10 μg to about 1000 μg of the one or more antigenic proteins and a priming virus that does not comprise a transgene or a nucleic acid sequence that encodes and expresses the antigenic protein.

In certain embodiments of the sequential heterologous boost methods presented herein, a dose of a priming composition is administered to a subject about 7 to about 90 days immediately prior to the administration of a first boost dose to the subject. In particular embodiments, a dose of a priming composition is administered to a subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days immediately prior to the administration of a first boost dose to the subject. For example, in certain embodiments of the sequential heterologous boost methods presented herein, a dose of a priming composition is administered to a subject about 7 to about 90 days immediately prior to the administration of a first boost dose to the subject. In particular embodiments, a dose of a priming composition is administered to a subject about 7 to about days, 14 to about 60 days, about 14 to about 28 days, about 28 to about 60 days, about 14 days, about 15 days, about 21 days, about 28 days, about 29 days, about 30 days, about 50 or about 60 days immediately prior to the administration of a first boost dose to the subject. In particular embodiments, a second, heterologous boost dose is administered to the subject about 2 weeks to about 3 months after the first boost dose is administered to the subject.

In particular embodiments, the first boost dose is administered to the subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days after the dose of the priming composition is administered to the subject. In particular embodiments, the first boost dose is administered to the subject about 2 weeks to about 4 weeks, about 2 weeks to about 8 weeks, about 2 weeks to about 12 weeks, about 2 weeks, about 3 weeks, or about 4 weeks after the dose of the priming composition is administered to the subject. In particular embodiments, the first boost dose is administered to the subject about 2 weeks to about 3 months after the dose of the priming composition is administered to the subject. In particular embodiments that utilize a dose of a priming composition that comprises an adoptive cell transfer of antigen-specific CD8+ T cells, the first boost dose may be administered to the subject about 1 to about 7 days after the dose of the priming composition.

In certain embodiments, a prime dose may be administered as a single composition. In other embodiments, a prime dose may be administered in parts. When a prime dose is administered in parts, e.g., 2, 3, or 4 parts, the parts may be administered concurrently or sequentially. Administration of a prime dose is complete prior to the initiation of the administration of the first boost dose.

In certain embodiments, administration of prime dose is performed intravenously, intramuscularly, intraperitonealy, or subcutaneously. In a particular embodiment, administration of a prime does is performed intravenously. In instances where a prime dose is administered in parts, the parts may be administered by the same or different routes of administration.

In certain embodiments of the sequential heterologous boost methods presented herein, the dose of one or more of the boosts comprises about 1×10⁷ particle forming units (PFU) to about 5×10′² PFU of oncolytic virus. In certain embodiments, the dose of the first boost comprises an about 10-fold to an about 100-fold higher amount of oncolytic virus than the dose of the subsequent boost(s). In particular embodiments, the oncolytic virus comprises a nucleic acid that expresses, in a subject, antigenic protein, as described herein. In other particular embodiments, the oncolytic virus is an oncolytic virus that does not comprise a nucleic acid that expresses the antigenic protein, as described herein.

In certain embodiments wherein a boost dose comprises one or more proteins capable of inducing an immune response to one or more neoantigens of interest, that is, comprises one or more antigenic proteins, the dose of such a boost dose may comprise about 10 μg to about 1000 μg of the one or more antigenic proteins. In particular embodiments, these amounts refer to the amount of antigenic protein present in a boost dose in the aggregate. In other particular embodiments, these amounts refer to the amount of each antigenic protein present in the boost dose.

In certain embodiments, one or more boost doses may be administered as a single composition. In other embodiments, each of the boost doses may be administered as a single composition. In certain embodiments, any of the boost doses may be administered in parts. In other embodiments, each of the boost doses may be administered in parts. In still other embodiments, a first boost dose may be administered in parts, and subsequent boost doses are administered as a single composition. When a boost dose is administered in parts, e.g., 2, 3, or 4 parts, the parts may be administered concurrently or sequentially. Administration of a boost dose is complete prior to the initiation of the administration of the next consecutive boost, if any.

In instances where a prime dose is administered in parts, the timing of the administration of the first dose may be measured from the administration of any of the parts of the prime dose. For example, in instances where the prime dose is administered in parts and the parts are administered sequentially, the timing of the administration of the first boost dose may be measured from the administration of the first part of the prime dose or, e.g., from the administration of the final part of the prime dose. In instances where a first boost dose is administered in parts, generally the timing of administration of the first boost dose is measured from the initiation of the first boost, that is, from the administration of the first part of the boost dose.

In certain embodiments of the sequential heterologous boost methods presented herein, a boost dose is administered to a subject about 7 to about 90 days after the immediately prior boost dose is administered to a subject. In particular embodiments, a boost dose is administered to the subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days after an immediately prior dose is administered to the subject. For example, in certain embodiments of the sequential heterologous boost methods presented herein, a second, heterologous boost dose is administered to a subject about 7 to about 90 days after the first boost dose is administered to a subject. In particular embodiments, a second, heterologous boost dose is administered to the subject about 7 to about days, 14 to about 60 days, about 14 to about 28 days, about 28 to about 60 days, about 14 days, about 15 days, about 21 days, about 28 days, about 29 days, about 30 days, about 50 or about 60 days after the first boost dose is administered to the subject. In particular embodiments, a second, heterologous boost dose is administered to the subject about 2 weeks to about 3 months after the first boost dose is administered to the subject.

In other particular embodiments, boosts are administered using a cycle that leaves about 28 days, 30 days, or 60 days between boosts. In one such embodiment, the cycle alternates use of a boost comprising a first oncolytic virus followed by a second oncolytic virus and leaves about 28 days, 30 days, or 60 days between boosts. In one example of such a cycle, one boost comprises a Farmington virus and the other boost comprises a Maraba virus, e.g., an MG1 virus. In another example of such a cycle, one boost comprises a Farmington virus and the other boost comprises a vaccinia virus, e.g., a CopMD5p, CopMD3p, or CopMD5p3p vaccinia virus. In another example of such a cycle, one boost comprises a Farmington virus and the other boost comprises a CopMD5p3p vaccinia virus with a B8R deletion. In yet another example of such a cycle, one boost comprises a Maraba virus, e.g., an MG1 virus, and the other boost comprises a vaccinia virus, e.g., a CopMD5p, CopMD3p, or CopMD5p3p vaccinia virus. In yet another example of such a cycle, one boost comprises a Maraba virus, e.g., an MG1 virus, and the other boost comprises a CopMD5p3p vaccinia virus with a B8R deletion.

In certain embodiments of the sequential heterologous boost methods presented herein, a boost dose is administered to a subject about 2 weeks to about 8 weeks after the immediately prior boost dose is administered to a subject. In particular embodiments, a boost dose is administered to the subject about 2 weeks to about 4 weeks, about 2 weeks to about 8 weeks, about 2 weeks to about 12 weeks, about 2 weeks, about 3 weeks, or about 4 weeks after the immediately prior boost dose is administered to the subject. For example, in certain embodiments of the sequential heterologous boost methods presented herein, a second, heterologous boost dose is administered to a subject about 2 weeks to about 8 weeks after the first boost dose is administered to a subject. In particular embodiments, a second, heterologous boost dose is administered to the subject about 2 weeks to about 4 weeks, about 2 weeks to about 8 weeks, about 2 weeks to about 12 weeks, about 2 weeks, about 3 weeks, or about 4 weeks after the first boost dose is administered to the subject.

In instances where an immediately prior boost is administered in parts, the timing of the administration of the immediately prior boost dose may be measured from the administration of any of the parts of the immediately prior boost dose. For example, in instances where the immediately prior boost dose is administered in parts and the parts are administered sequentially, the timing of the administration of the immediately prior boost dose may be measured from the administration of the first part of the immediately prior dose or, e.g., from the administration of the final part of the immediately prior dose. In instances involving the timing between two consecutive boosts wherein at least the later of the two consecutive boosts is administered in parts, generally the timing of the administration of the later of the two consecutive boost doses is measured from the initiation of the later boost, that is, from the administration of the first part of the later boost dose.

In certain embodiments, administration of at least one boost dose is performed intravenously, intramuscularly, intraperitonealy, or subcutaneously. In a particular embodiment, at least one boost dose is performed intravenously. In particular embodiments, each of the boost doses is performed intravenously. In instances where a boost dose is administered in parts, the parts may be administered by the same or different routes of administration.

In a specific embodiment, the methods of inducing an immune response to one or more neoantigens described herein treat the subject's cancer. In some embodiments, a method of inducing an immune response to one or more neoantigens described herein results in one, two, three or more of the following effects: complete response, partial response, objective response, increase in overall survival, increase in disease free survival, increase in objective response rate, increase in time to progression, stable disease, increase in progression-free survival, increase in time-to-treatment failure, and improvement or elimination of one or more symptoms of cancer. In a specific embodiment, a method of inducing an immune response to one or more neoantigens described herein results in an increase in overall survival of the subject. In another specific embodiment, a method of inducing an immune response to one or more neoantigens described herein results in an increase in progression-free survival of the subject. In another specific embodiment, a method of inducing an immune response to one or more neoantigens described herein results an increase in overall survival of the subject and an increase in progression-free survival. In a specific embodiment, the methods of inducing an immune response to one or more neoantigens described herein may result in a decrease in tumor burden from baseline (e.g., 10%, 15%, 20%, 25%, 30%, 35%, 40%, 45%, 50%, 55% or more, or 10% to 25%, 25% to 50%, or 25% to 75% decrease in tumor burden from baseline).

### Kits

In one aspect, provided herein is a pharmaceutical pack or kit comprising one or more components necessary to practice a heterologous boost method described herein. In one embodiment, provided herein is a pharmaceutical pack or kit comprising a composition(s) for first boost composition and a composition(s) for a second boost, wherein the composition or the components of each composition for each boost may be in a separate container. In another embodiment, provided herein is a pharmaceutical pack or kit comprising compositions for two or more boosts described herein, wherein the compositions or the components of each composition for each boost may be in a separate container. In another embodiment, provided herein is a pharmaceutical pack or kit comprising a priming composition and compositions for two or boosts described herein, wherein the compositions or the components of each composition for each boost and priming composition may be in a separate container. In a specific embodiment, the pack or kit further comprises instructions for each of the compositions in the heterologous boost method described herein. In some embodiments, the pack or kit further one or more components: (1) to determine if the subject has pre-existing immunity to a neoantigen, (2) to assess the immune response induced following one or steps of a heterologous boost method described herein, or (3) both (1) and (2).

### EXAMPLES

### Example 1

### Materials and Methods

Mouse Models. All animal procedures were performed in accordance with the institutional guidelines of the University of Ottawa committee on the Use of Live Animals in Teaching and Research in accordance with guidelines established by the Canadian Council on Animal Care.

Six- to eight-week C57BL/6 female mice were purchased from Charles River Canada (Constant, QC, Canada) and allowed to acclimatize for at least one week prior to the study start date. No special diet was used for any study. Mice were kept in sterile isolation cages and maintained on a 12-hr dark-light cycle.

Naïve Mice. 7-10 weeks old female C57BL/6 mice were primed at day 0 with either adenovirus (AdV) expressing various transgenes by bilateral intramuscular injection; or one or more peptides at 50 μg intraperitoneally (IP) or subcutaneously (SC) (Biomer Technology) with adjuvant: 30 μg of anti-CD40 antibody (BioXCell) and 10 μg of poly I:C (manufacturer unknown); or liposome-wrapped peptide (1 μg) or liposome-wrapped mRNA nanoparticles. Mice were boosted intravenously with 3×10⁸ PFU FMT or MG1 virus expressing MC-38-derived (Adpgk, Repsl, Irgq, Cpnel, Aatf) plus B16.F10-derived (Obsl1, Snx5, Pbk, Atp1 1a, Eef2) neoantigens in a conventional random order (FMT-N10 or MG1-N10) or an algorithm-optimized order (MG1-N10opt) or a fusion ORF (MG1-N10fusion); or virus with no reporter gene (FMT-nr or MG1-nr) and one or more peptides (1-100 μg) administered IV (mixed with the virus) or SC. In the case of Maraba virus, e.g., MG1 virus, the nucleic acids expressing the antigenic proteins are inserted into the Maraba genome between the G and L gene sequences. In the case of Farmington virus, e.g., FMT virus, the nucleic acids expressing the antigenic proteins are inserted into the Farmington genome between the N and P gene sequences. Non-terminal peripheral blood samples were collected at specific days following the first boost and second boost and in some cases at later time points for quantification of antigen-specific T cells by ex vivo peptide stimulation and intracellular cytokine staining (ICS) assay.

Rhabdovirus Titration. Rhabdoviruses were titred on Vero cells seeded into 6-well plates (5×10⁵ cells per well). The next day 100 μl of serial viral dilutions were prepared and added for 1 hour to Vero cells. After viral adsorption, 2 ml of agarose overlay was added (1:1, 1% agarose:2× Dulbecco's modified Eagle's medium and 20% FCS). Plaques were counted the following day. Where applicable, diameters were measured and plaque area calculated using the following formula Area=πr².

Optimizing Neoantigen Order. For rhabdovirus vectors, an optimized order of ten neoantigens in the beads-on-a-string vaccine configuration was determined using a modified version of OptiVac 1.0. This software evaluates the likelihood of epitope recovery by ordering the different epitopes in every possible combination and inserting spacers between the junctions. To ensure proper recovery, the cleavage sites must be located in the spacers between each epitope. (Schubert B, Kohlbacher 0. Designing string-of-beads vaccines with optimal spacers. Genome medicine. 2016; 8(1):9) Our algorithm was customized by changing the spacer sequence from the original (H)nA sequence to an AAY spacer peptide. Proteasomal cleavage sites were validated to ensure that change in spacer sequence did not affect the recovery of epitopes.

Generating Fusion Neoantigen Cassettes. Fusion neoantigen cassettes were built into MG1 between G and L proteins. These comprised of a Kozak sequence upstream of a human ubiquitin sequence (1 to 76; codon 76 G>V) followed by a flexible linker, a proteosomal AAY cleavage sequence, ten 27mer codon-optimized neoantigens with no intervening sequence and a stop codon.

Recombinant Human Adenovirus 5 Priming. Mice were anaesthetized, and the hind legs were swabbed with 70% Ethanol. Recombinant human adenovirus serotype 5 was administered IM at a dose of 2×10⁸ PFU split bilaterally between both hind legs.

Peptide Priming. Single peptides were administered IP or SC at a dose of 50 μg in 250-600 μL DPBS together with 30 μg anti-CD40 and 10 μg poly LC per mouse. Multiple peptides were administered IP at an individual peptide dose of 50 μg in 250-600 μL DPBS IP or SC together with 30 μg anti-CD40 and 10 μg poly LC per mouse.

Rhabdovirus Booster Vaccines. Rhabdoviruses were diluted in order to deliver 3×10⁸ PFU per mouse in 100 μL DPBS. Mice were placed in a restrainer, and the tail was immersed in warm water or under a heat lamp until the vein is visible. 70% ethanol was used to swab the tail, and mice were then injected with 100 μL of virus (corresponding to a dose ranging from 3×10⁸ PFU) IV via the tail vein.

For experiments involving FMT-nr or MG1-nr boosts/superboosts in the presence of loose peptides, loose peptides were administered at 50 μg per peptide in 200-600 μL SC (separately from virus) or 100-200 μl IV (mixed with virus).

Flow Cytometry Antibodies. The following antibodies used for flow cytometry were purchased from BD Biosciences: anti-CD8α (clone 53-6.7); anti-IFN-γ (clone XMG1.2); anti-TNF-α (clone MP6-XT22); anti-IL-2 (clone JES6-5H4). Fixable viability dye (eFluor 780 or eFluor450) was purchased from eBioscience. Results from stained samples were acquired using a LSR (BD Biosciences) and analyzed using FlowJo (Tree Star, Ashland, Oreg.).

Preparation of Tissues for Flow Cytometry. Non-terminal peripheral blood samples were collected from the saphenous vein into heparinized tubes (Microvette CB 300; SARSTEAD AG&Co). Blood was stored overnight at 4° C. prior to processing or processed immediately. Red blood cells were removed by treatment with a 0.15 mol/1 NH₄C1 lysis buffer (pH 7.4). The isolated peripheral blood mononuclear cells (PBMCs) were resuspended in RPMI-10 medium and used for further downstream experiments.

Intracellular Cytokine Staining (ICS). PBMCs suspended in complete RPMI were added to round-bottom 96-well plates and restimulated with 5 μg/ml of peptide (5×MC-38 peptides: Adpgk (ASMTNMELM, SEQ ID NO: 1), Repsl (AQLANDVVL, SEQ ID NO: 2), Irgq (AALLNSAVL, SEQ ID NO: 3), Cpnel (SSPYSLHYL, SEQ ID NO: 4), Aaltf (MAPIDHTTM, SEQ ID NO: 5); 5×B16.F10 peptides: Obsl1 (LCPGNKYEM, SEQ ID NO: 6), Snx5 (R373Q) (AAFQKNLIEM, SEQ ID NO: 7), Pbk (AAVILRDAL, SEQ ID NO: 8), Atp1 1a (QSLGFTYL, SEQ ID NO: 9) and Eef2 (VKAYLPVNESFAFTA, SEQ ID NO: 10); 1 μg/m1Maraba N₅₂₋₅₉ peptide (RGYVYQGL, SEQ ID NO: 11; C57BL/6 mice); or FMT N₃₀₁₋₃₀₉ (AVVLMFAQC, SEQ ID NO: 12)) for 5 hours at 37° C. Negative (unstimulated) controls received DMSO in RPMI. Positive control wells received PMA (100 ng/ml) plus ionomycin (1 μg/ml). After 1 hour, Brefeldin A (0.2 μl/well; BD Biosciences) was added to each well. After stimulation, cells were washed with normal RPMI medium containing 10% FCS and resuspended back in this medium and stored overnight at 4° C. The next day, cells were washed twice with 0.5% BSA in PBS (FACS buffer) and incubated at 4° C. for 15 minutes with Fc block (Clone 2.4G2; BD Biosciences) diluted in FACS buffer. Cells were stained with live/dead cell marker and surface markers for 30 minutes at 4° C., then permeabilized with Cytofix/Cytoperm (BD Biosciences) according to the manufacturer's instructions. Anti-IFN-γ, anti-TNF-α, and anti-IL-2 were incubated with the samples for 30 minutes at 4° C. and cells were then washed in Perm/Wash buffer (BD Biosciences). Samples were resuspended in FACS buffer for analysis. Results are presented as frequency or numbers per ml of blood of cytokine-positive cells per total CD8⁺ T cells following peptide stimulation minus the same values obtained in control (unstimulated) samples.

Results are presented as frequency of cytokine-positive cells per total CD8+ T cells following peptide stimulation minus the same values obtained in control (unstimulated) samples, or number of cytokine-positive CD8+ T cells.

Data were acquired on BD LSR Fortessa X20 flow cytometer with HTS unit (BD Biosciences) and data were analyzed using FlowJo (TriStar) software. The debris and doublets were excluded by gating on FSC vs SSC and FSC-A vs FSC-H, respectively. Viable cells were gated based on viability dye stain. Next, CD8-positive cells were gated and within this population the expression of IFNγ, TNFα and IL-2 was examined. Cell numbers were calculated with the following formula:

$$N [ cell number \text{/} ml ] = \frac{N s - N u}{( \frac{V m}{W} ) * V f} * 1000$$

where N—resulting positive cell number per 1 ml of blood, Ns—number of positive cells in the well containing peptide, Nu—number of positive cells in unstimulated control, Vm—total blood volume collected from animal, W—number of wells the blood sample was distributed into, Vf—fraction of sample volume used for data acquisition by flow cytometry i.e., 80 μl out of 130 μl.

Statistics. For plaque size determinations, one-way analysis of variance was performed using the Bonferroni multiple comparison's test to derive a P value. For Kaplan-Meier plots, we compared survival plots using Mantel-Cox log-rank analysis. Titers and viability were compared using a two-tailed unpaired Student's T test to derive a P value. When 2 different groups were compared for one variable, the t-test (Mann-Whitney test) was used. When more than 2 different groups were compared for one variable, the one-way ANOVA (Kruskal-Wallis) test with Dunn's multiple comparison test was used. When 2 or more groups were compared for 2 variables (ex. post boost 1 and post boost 2) the two-way ANOVA test with multiple comparison test was used. Results reported using the following symbols: * p-value <0.05, ** p-value <0.01, *** p-value <0.001 and **** p-value <0.0001. All comparisons were performed using either Graphpad Prism (Graphpad Software, La Jolla, Calif.) or Microsoft Excel.

### Results

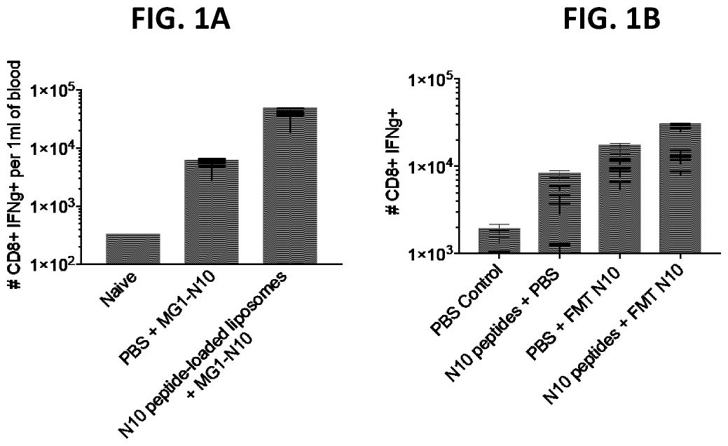

Medium Payloads. Both FMT and MG1 can accommodate large transgenes capable of encoding multiple neoantigen targets. FMT in particular has high genome flexibility and capacity. Rhabdovirus vectors encoding medium-sized multi-neoantigen transgene payloads can boost CD8+ T cell responses against multiple neoantigen targets. Rhabdoviruses encoding ten-neoantigen cassettes (five neoantigens from the MC-38 tumour model, and five neoantigens from the B16.F10 tumour model) can boost CD8+ T cells to each individual neoantigen to large frequencies (MG1: FIG. 1A; and FMT: FIG. 1B).

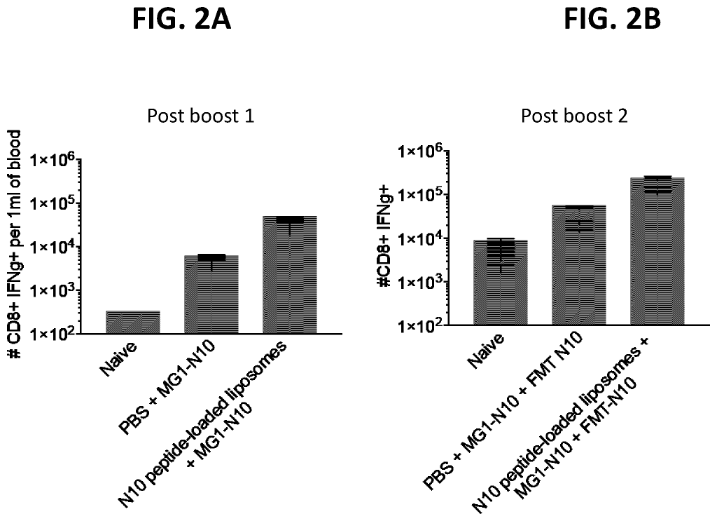

Oncolytic rhabdovirus vaccines can prime CD8+ T cell responses against multiple encoded neoantigen targets that can subsequently be ‘superboosted’ by a second heterologous oncolytic rhabdovirus. Boosted CD8+ T cell responses against multiple neoantigen targets can be ‘superboosted’ to several hundred-fold higher frequencies through the administration of a second heterologous oncolytic rhabdovirus vaccine (FIGS. 2A-2B). Since the size of the CD8+ T cell response is correlated with improved survival (Strickland et al. “Association and prognostic significance of BRCA1/2-mutation status with neoantigen load, number of tumor-infiltrating lymphocytes and expression of PD-1/PD-L1 in high grade serous ovarian cancer.” Oncotarget. 2016; 7(12):13587-13598; van Poelgeest et al. “Vaccination against Oncoproteins of HPV16 for Noninvasive Vulvar/Vaginal Lesions: Lesion Clearance Is Related to the Strength of the T-Cell Response.” Clin Cancer Res. 2016; 22(10):2342-2350), the ability to use a multi-oncolytic vaccination approach that progressively increases the size of the CD8+ T cell pool against multiple neoantigen targets represents a key feature of future clinical protocols.

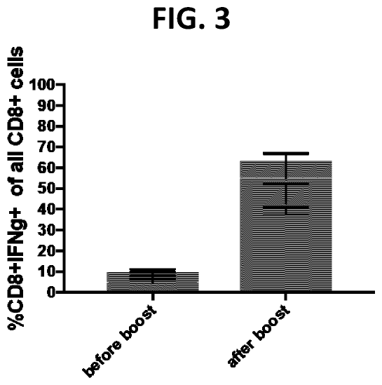

Heterologous boost with rhabdovirus can engage neoantigen-specific CD8+ T cells. Several priming technologies can be paired with an oncolytic booster vaccine to expand CD8+ T cells against multiple tumour neoantigen targets. This includes (but is not limited to) recombinant replication-incompetent human adenovirus serotype 5 (FIG. 3).

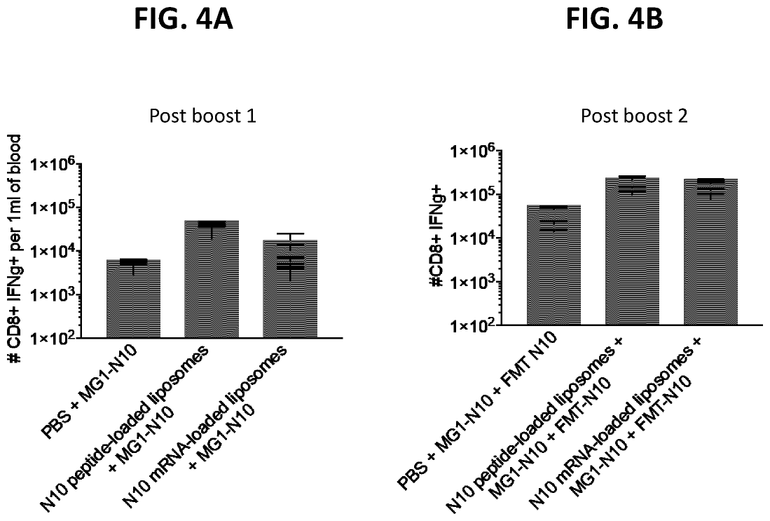

Nanoparticle technologies can also be used to engage and superboost a robust CD8+ T cell response against multiple neoantigen targets (FIGS. 4A-4B). Using a dual liposome-wrapped peptide prime, CD8+ T cells recognizing ten neoantigen epitopes can be substantially expanded by both the initial boost (MG1-N10) and the superboost (FMT-N10) (FIGS. 4A-4B). A dual liposome-wrapped mRNA prime can generate CD8+T cells that can be boosted and superboosted against multiple neoantigen targets (FIGS. 4A-4B).

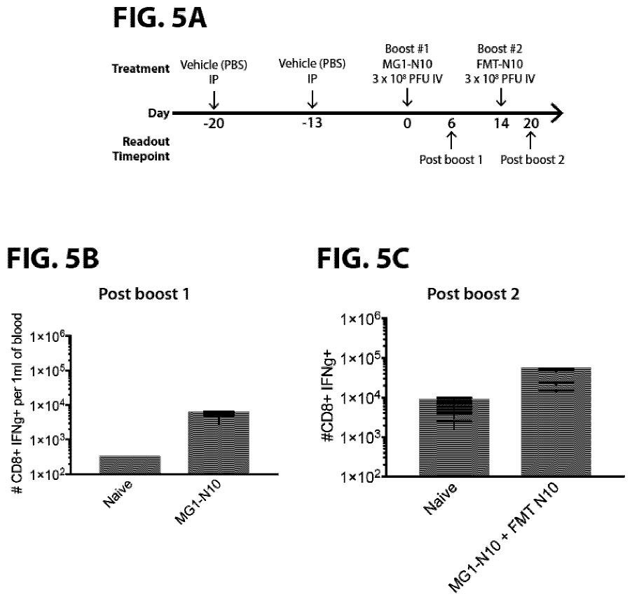

A pool of tumour-specific CD8+ T cells can be boosted by a second oncolytic heterologous rhabdovirus (FIG. 5A). In the neoantigen setting, vaccination with MG1 encoding ten tumour neoepitopes (MG1-N10) can establish an immunological memory CD8+ T cell pool that can be amplified by FMT-N10 vaccination (FIGS. 5B-5C). This indicates that, regardless of the level of the prime (if a priming vaccination is used as part of a clinical protocol), superboost will still have a significant impact on expanding large frequencies of tumour-specific CD8+ T cells. It also indicates that future clinical protocols could be streamlined to remove any formal priming vaccination to focus solely on a multi-oncolytic virus vaccine therapy.

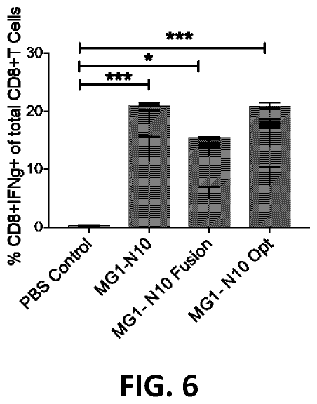

Multi-neoantigen cassettes can be encoded in different genetic configurations to direct hierarchical CD8+ T cell responses. Multiple neoantigens can be encoded in different transgene configurations not only to improve the magnitude of the CD8+ T cell response, but also to direct CD8+ T cell responses against the highest priority neoantigen targets. In a comparison with the core MG1 vaccine vector, MG1-N10, two different transgene configurations ((1) MG1-N10fusion, where ten neoantigens are fused randomly in a single open reading frame; or (2) MG1-N10-Opt, where the position of the ten neoantigens is optimized according to a pre-defined algorithm) substantially modulate the proportion of the total CD8+ T cell response that reacts against specific key neoantigen targets (FIG. 6, Table 1). For example, CD8+ T cell responses against Reps1 can be prioritized in the MG1-N10 fusion vector, while responses against Atp1 1a and Eef2 can be prioritized in the MG1-N10-opt vector (Table 1).

This is an important technological advance in the personalized medicine field, since most neoantigen prediction pipelines generate priority lists of several high value neoantigens based on predicted MHC binding affinities. It is therefore highly beneficial to be able to activate and direct CD8+ T cell responses against neoantigen targets according to a pre-defined priority hierarchy.

TABLE 1
Proportion of the Response Against Each individual
neoantigen target by the virus transgene technology employed. All
virus transgenes were encoded into the same MG1 backbone.

| | Virus Transgene Technology | | |
|---|---|---|---|
| Neoantigen | MG1-N10 | MG1-N10 Fusion | MG1-N10 Opt |
| Adpgk | 54.61 | 32.98 | 35.29 |
| Reps1 | 30.78 | 48.80 | 32.42 |
| Irgq | 7.56 | 4.41 | 9.42 |
| Cpne1 | 1.56 | 3.32 | 2.87 |
| Aaltf | 0.96 | 0.39 | 0.00 |
| Obsl1 | 0.00 | 0.00 | 0.00 |
| Snx5 | 0.18 | 3.08 | 4.80 |
| Pbk | 3.46 | 1.61 | 3.22 |
| Atp11a | 0.21 | 4.65 | 7.24 |
| Eef2 | 0.69 | 0.76 | 4.75 |
| Total Proportion (%) | 100 | 100 | 100 |

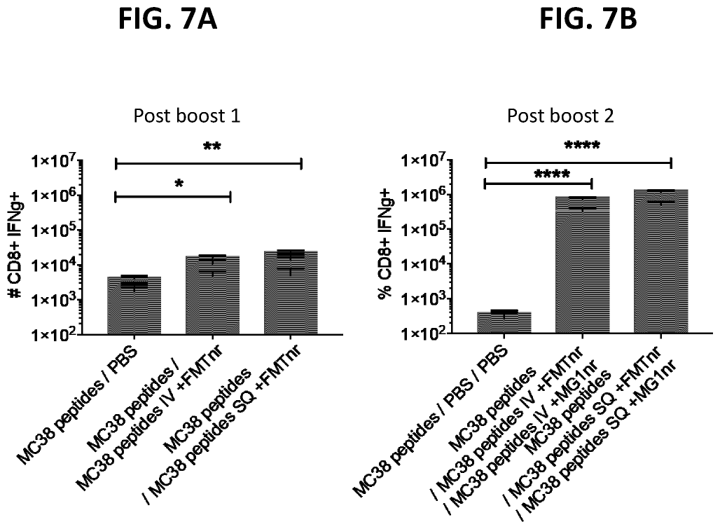

In addition to boosting or superboosting CD8+ T cell responses against multiple neoantigen targets via specifically genetically encoded transgene cassettes, oncolytic rhabdovirus vectors without any encoded vaccine transgenes can also drive substantial CD8+ T cell responses against the same neoantigen targets when co-administered with loose peptides. Loose peptides can be administered by either subcutaneous or intravenous routes to achieve similarly robust boosting of CD8+ cell responses against multiple neoantigen targets (FIGS. 7A-7B).

This represents a substantial technological advance towards personalized oncolytic virus vaccination, since neoantigen targets do not have to be encoded into the viral genome and therefore viruses do not have to be made on a per-patient basis. Instead, a single ‘empty’ virus can be administered along with a pre-defined pool of specific neoantigen peptides to enable to an individualized response. Ultimately, this pairs personalized (private) neoantigen peptides with a universal (public) virus and consequently streamlines the entire clinical manufacturing process.

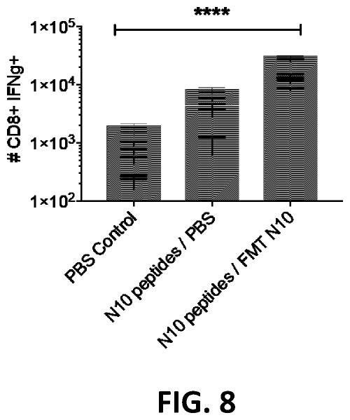

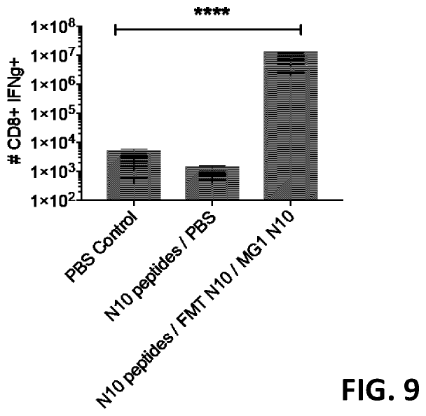

FIG. 8 shows the numbers of CD8+ IFN-γ positive cells of CD8+ T cells obtained following a prime with PBS or loose peptides (N10) and a boost with PBS or 3×10⁸ PFU of FMT N10 (FMT encoding 10 peptides). FIG. 9 shows the numbers of CD8+ IFN-γ positive cells of CD8+ T cells obtained at day 34 after mice were primed with PBS or loose peptides (N10), administered a first boost with PBS or 3×10⁸ PFU of FMT N10 (FMT encoding 10 peptides), and administered a second boost with 3×10⁸ PFU of MG1 N10 (MG1 encoding 10 peptides).

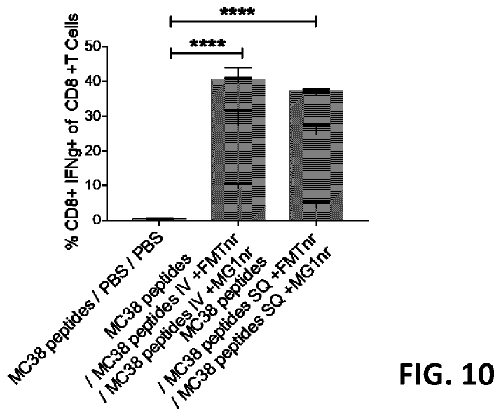

FIG. 10 shows the percentage of CD8+ IFN-γ positive cells of CD8+ T cells obtained 32 days after mice received a second boost with 3×10⁸ PFU of MG1 nr plus MC38 SC or MC38 IV. Mice were primed with adjuvanted MC38 subcutaneously (SC), administered a first boost with PBS or 3×10⁸ PFU of FMT nr plus MC38 IV or MC38 SC, and administered a second boost with 3×10⁸ PFU of MG1 nr plus MC38 IV or MC38 SC.

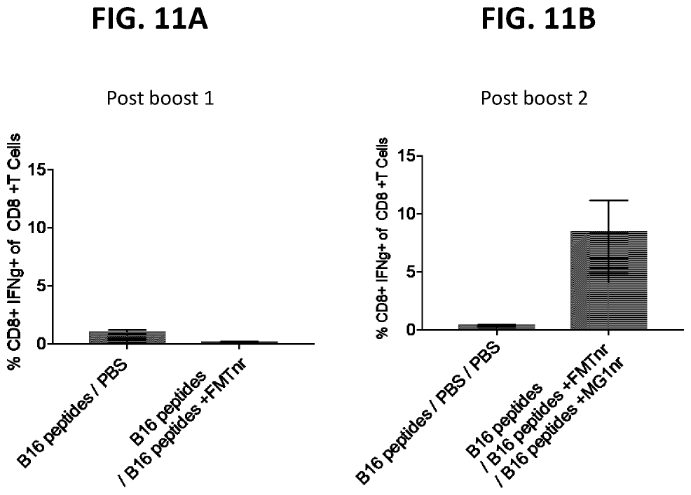

FIG. 11 shows the percentage of CD8+ IFN-γ positive cells of CD8+ T cells obtained following a prime with adjuvanted loose B16 subcutaneously, a first boost with PBS, or 3×10⁸ PFU of FMT NR IV plus B16 IV, and a second boost with PBS, or 3×10⁸ PFU of MG1 nr IV plus B16 IV.

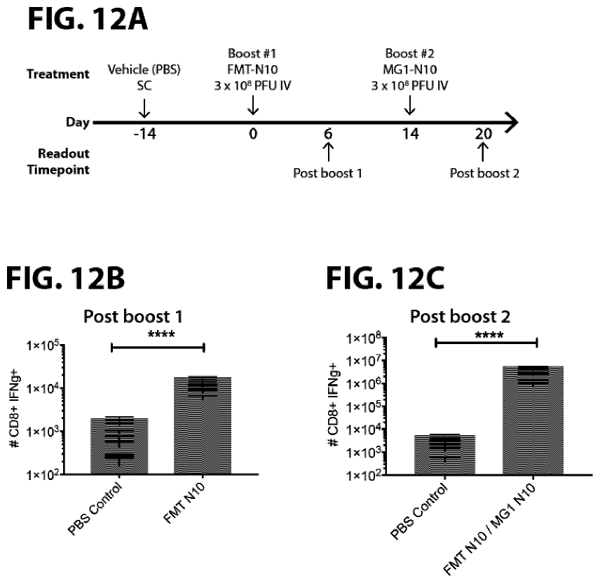

As shown in FIGS. 12B and 12C, boosting responses against multiple neoantigen targets does not require a formal prime. Naive mice received vehicle (PBS) followed by FMT-N10 and MG1-N10 boosts. Non-terminal peripheral blood samples were sampled, stimulated with the corresponding 10 neoantigen peptides, and analyzed by intracellular cytokine staining. See FIG. 12A for the experimental protocol and timeline for the data presented in FIGS. 12B and 12C.

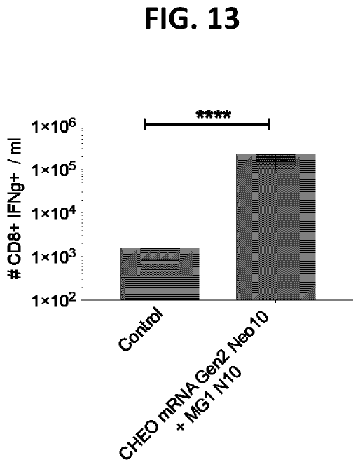

As shown in FIG. 13, a boost can engage CD8+ T cells established by mRNA nanoparticle priming technology.

### Example 2

Generating effective T cell mediated clearance of solid tumors remains an unmet challenge in cancer therapy. Suppressive tumor microenvironments limit the generation of significant numbers of tumor specific T effector cells, their migration to tumor beds, and their subsequent functionality within tumors, thereby blocking the patient's normally potent acquired immune response from contributing to tumor control.

In an effort to meet this challenge, we have developed novel oncolytic viruses that were bioselected and engineered to cause cancer cell death through two distinct and complementary mechanisms-of-action, direct cancer lysis and tumor-antigen specific T cell generation. Specifically, we have selected and engineered MG1 Maraba virus which both infects tumor tissue to reverse immune suppressive programs while simultaneously delivering vector encoded tumor antigens to the spleen to vaccinate against the patient's tumor. The result is a large increase in peripheral and tumor infiltrating CD8+T effectors cells, strong intratumoral inflammatory signatures and ultimately curative efficacy in preclinical solid tumor models. This first-in-class therapeutic strategy is currently being evaluated in Phase 1 and Phase 2 clinical trials.

In this new study, we describe the development of a novel viral immunotherapy platform based on Farmington virus that is: (1) oncolytic in solid tumor models, (2) a potent inducer of highly-functional antigen-specific T cells (expanding tumor specific CD8+ T effector pools over 1000 fold from pre-existing T central memory) and is (3) immunologically distinct from our clinical MG1 Maraba platform. We show that when used sequentially in a heterologous boosting regimen, T cell responses to encoded multi-neoantigens, can exceed greater than 50% of all CD8+ T cells in the periphery. The majority of these CD8+ T effectors show markers of polyfunctionality with little expression of the PD-1 exhaustion marker. We will describe our current data assessing the phenotype, localization and potency of these T cell responses in preclinical models of solid tumors and propose strategies to deploy our novel dual oncolytic viral immunotherapy boosting paradigm to the clinic.

### EQUIVALENTS

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method of inducing an immune response to at least one neoantigen in a subject with pre-existing immunity to the at least one neoantigen, or a subject who has previously been administered a dose of a priming composition that is capable of inducing an immune response to the at least one neoantigen, the method comprising:
(a) administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen; and
(b) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the second oncolytic virus is immunologically distinct from the first oncolytic virus, and wherein the third and fourth compositions are administered concurrently or sequentially to the subject.

2. A method of inducing an immune response to at least one neoantigen in a subject with pre-existing immunity to the at least one neoantigen, or a subject who has previously been administered a dose of a priming composition that is capable of inducing an immune response to the at least one neoantigen, the method comprising:
(a) administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and
(b) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

3. A method of inducing an immune response to at least one neoantigen in a subject, comprising administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the third and fourth compositions are administered concurrently or sequentially to the subject,
wherein the subject has pre-existing immunity to the at least one neoantigen, or the subject was previously administered a dose of a priming composition that is capable of inducing an immune response to the at least one neoantigen, and
wherein the subject was previously administered a first boost comprising a dose of a first composition, wherein the first composition comprises a first oncolytic virus comprising a genome that comprises a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

4. A method of inducing an immune response to at least one neoantigen in a subject, comprising administering to the subject a second boost comprising a dose of a fourth composition, wherein the fourth composition comprises a second oncolytic virus that comprises a genome comprising a first transgene, wherein the first transgene encodes and expresses a first protein that is expressed in the subject, wherein the first protein or a fragment thereof is capable of inducing an immune response to the at least one neoantigen,
wherein the subject has pre-existing immunity to the at least one neoantigen, or the subject was previously administered a dose of a priming composition that is capable of inducing an immune response to the at least one neoantigen, and
wherein the subject was previously administered a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject, wherein the first peptide composition is capable of inducing an immune response to the at least one neoantigen, and wherein the second oncolytic virus is immunologically distinct from the first oncolytic virus.

5. The method of claim 1, wherein step (b) is performed 7 to 21 days after step (a).

6. The method of claim 2, wherein step (b) is performed 7 to 21 days after step (a).

7. The method of claim 1, wherein step (b) is performed 2 weeks to 3 months after step (a).

8. The method of claim 2, wherein step (b) is performed 2 weeks to 3 months after step (a).

9. The method of claim 3, wherein the first boost was administered to the subject 7 to 21 days before the second boost.

10. The method of claim 4, wherein the first boost was administered to the subject 7 to 21 days before the second boost.

11. The method of claim 3, wherein first boost was administered to the subject 2 weeks to 3 months before the second boost.

12. The method of claim 4, wherein the first boost was administered to the subject 2 weeks to 3 months before the second boost.

13. The method of claim 1, 3, 5, 7, 9 or 11, wherein the second oncolytic virus comprises a genome that comprises a second transgene, wherein the second transgene encodes and expresses a second protein in the subject, and wherein the second protein or a fragment thereof is capable of inducing an immune response to the at least one neoantigen.

14. The method of claim 2, 4, 6, 8, 10, or 12, wherein the first oncolytic virus comprises a genome that comprises a second transgene, wherein the second transgene encodes and expresses a second protein in the subject, and wherein the second protein or a fragment thereof is capable of inducing an immune response to the at least one neoantigen.

15. The method of claim 1, 3, 5, 7, 9, 11 or 13, wherein the first composition is administered to the subject intravenously or intramuscularly.

16. The method of claim 1, 3, 5, 7, 9, 11, 13 or 15, wherein the second composition is administered to the subject intravenously or intramuscularly.

17. The method of claim 1, 3, 5, 7, 9, 11, 13, or 15, wherein the third composition is administered to the subject intravenously or intramuscularly.

18. The method of claim 1, 3, 5, 7, 9, 11, 13, 15 or 17, wherein the fourth composition is administered to the subject intravenously or intramuscularly.

19. The method of claim 2, 4, 6, 8, 10, 12 or 14, wherein the first composition is administered to the subject intravenously or intramuscularly.

20. The method of claim 2, 4, 6, 8, 10, 12 or 14, wherein the second composition is administered to the subject intravenously or intramuscularly.

21. The method of claim 2, 4, 6, 8, 10, 12, 14 or 20, wherein the third composition is administered to the subject intravenously or intramuscularly.

22. The method of any one of claims 2, 4, 6, 8, 10, 12, 14 and 19 to 21, wherein the fourth composition is administered to the subject intravenously or intramuscularly.

23. The method of any one of claims 1, 3, 5, 7, 9, 11, 13, and 15 to 18, wherein the fourth composition comprises a liposome or a nanoparticle.

24. The method of any one of claims 2, 4, 6, 8, 10, 12, 14, and 19 to 22, wherein the fourth composition comprises a liposome or a nanoparticle.

25. The method of any one of claims 1 to 24, wherein the subject was administered the priming composition 7 to 21 days before the first boost.

26. The method of any one of claims 1 to 24, wherein the subject was administered the priming composition 2 weeks to 3 months before the first boost.

27. The method of any one of claims 1 to 26, wherein the immune response to the at least one neoantigen that is induced in the subject comprises a peak immune response to the at least one neoantigen with the second boost that is at least 0.5 log higher than the peak immune response to the at least one neoantigen attained with the first boost.

28. The method of any one claims 1 to 27, wherein one month after the second boost the immune response to the at least one neoantigen remains higher that the peak immune response to the at least one neoantigen attained with the first boost.

29. The method of claim 27 or 28, wherein the immune response is measured by the number of antigen-specific interferon gamma-positive CD8+ T cells per ml of peripheral blood from the subject.

30. The method of any one of claims 1 to 29, wherein the priming composition comprises: (i) a nucleic acid sequence, wherein the nucleic acid sequence encodes and expresses a first priming protein in the subject, wherein the first priming protein or a fragment thereof is capable of inducing an immune response to the at least one neoantigen, (ii) a priming peptide composition, wherein the priming peptide composition is capable of inducing an immune response to the at least one neoantigen, (iii) an adoptive cell transfer of CD8+ T cells specific for the at least one neoantigen, (iv) a first priming virus that comprises a genome comprising a first priming transgene, wherein the first priming transgene encodes and expresses a second priming protein in the subject, wherein the second priming protein or a fragment thereof is capable of inducing an immune response to the at least one neoantigen, or (v) a second priming virus and a priming peptide composition, and wherein the first priming virus and the second priming virus are immunologically distinct from the first oncolytic virus.

31. The method of claim 30, wherein the first priming virus and the second priming virus are immunologically distinct from the second oncolytic virus.

32. The method of claim 30 or 31, wherein the priming composition further comprises an adjuvant.

33. The method of claim 32, wherein the adjuvant comprises poly I:C.

34. The method of any one of claims 30 to 33, wherein the priming composition further comprises a liposome or a nanoparticle.

35. The method of any one of claims 1 to 34, wherein the first protein or fragment thereof is capable of inducing an immune response to two or more different neoantigens.

36. The method of claim 35, wherein the first protein comprises at least one epitope of each of the two or more neoantigens.

37. The method of any one of claims 1 to 36, wherein the first protein encoded by the first transgene includes at least one proteasomal cleavage site.

38. The method of any one of claims 1 to 37, wherein the first protein encoded by the first transgene is a fusion protein.

39. The method of any one of claims 1 to 38, wherein the first peptide composition is capable of inducing an immune response to two or more different neoantigens.

40. The method of claim 39, wherein the first peptide composition comprises two peptides, wherein one of the peptides comprises at least one epitope of one of the neoantigens, and the other peptide comprises at least one epitope of the other neoantigen.

41. The method of any one of claims 1 to 40, wherein the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus.

42. The method of claim 41, wherein the third oncolytic virus and the fourth oncolytic virus are immunologically distinct from the first oncolytic virus.

43. The method of any one of claims 1 to 42, wherein the first oncolytic virus, the second oncolytic virus or both are attenuated.

44. The method of any one of claims 1 to 43, wherein the first oncolytic virus, the second oncolytic viruses, or both are rhabdoviruses.

45. The method of any one of claims 1 to 43, wherein the first oncolytic virus or the second oncolytic virus is a vaccinia virus, an adenovirus, a measles virus, or a vesicular stomatitis virus.

46. The method of claim 45, wherein the vaccinia virus is Copenhagen, Western Reserve, Wyeth, Tian Tan or Lister.

47. The method of any one of claims 1 to 43, wherein the first or second oncolytic virus is a Maraba virus.

48. The method of claim 47, wherein the Maraba virus is MG1.

49. The method of any one of claims 1 to 43, wherein the first or second oncolytic virus is a Farmington virus.

50. The method of any one of claims 1 to 43, wherein the first oncolytic virus is a Maraba virus and the second oncolytic virus is a Farmington virus.

51. The method of any one of claims 1 to 43, wherein the first oncolytic virus is a Farmington virus and the second oncolytic virus is a Maraba virus.

52. The method of any one of claims 1 to 43, wherein the first oncolytic virus is a vaccinia virus and the second oncolytic virus is a Maraba virus.

53. The method of any one of claims 1 to 43, wherein the first oncolytic virus is a Maraba virus and the second oncolytic virus is a vaccinia virus.

54. The method of any one of claims 1 to 43, wherein the first oncolytic virus is a vaccinia virus and the second oncolytic virus is a Farmington virus.

55. The method of any one of claims 1 to 43, wherein the first oncolytic virus is a Farmington virus and the second oncolytic virus is a vaccinia virus.

56. The method of any one of claims 52 to 55, wherein the vaccinia virus is Copenhagen, Western Reserve, Wyeth, Tian Tan or Lister.

57. The method of any one of claims 1 to 56, wherein the subject has been determined to have pre-existing immunity to the at least one neoantigen.

58. The method of claim 57, wherein the subject is determined to have pre-existing immunity by measuring the number of antigen-specific interferon gamma-positive CD8+ T cells per ml of peripheral blood from the subject.

59. The method of any one of claims 1 to 56, wherein the subject was previously administered a dose of the priming composition.

60. The method of claim 59, wherein the subject was administered the dose of the priming composition 7 to 21 days before the subject was administered the first boost.

61. The method of claim 59, wherein the subject was administered the dose of the priming composition 2 weeks to 3 months before the subject was administered the first boost.

62. A method of inducing an immune response to at least one neoantigen in a subject with pre-existing immunity to the neoantigen, or a subject who has previously been administered a dose of a priming composition that is capable of inducing an immune response to the at least one neoantigen, the method comprising:
(a) administering to the subject a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject; and
(b) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the fifth and sixth compositions are administered concurrently or sequentially to the subject, wherein the first and second peptide compositions are each capable of inducing an immune response to the at least one neoantigen, and wherein the second oncolytic virus is immunologically distinct than the first oncolytic virus.

63. A method of inducing an immune response to at least one neoantigen in a subject, comprising administering the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the fifth and sixth compositions are administered concurrently or sequentially to the subject,
wherein the subject has pre-existing immunity to the at least one neoantigen, or the subject was previously administered a dose of a priming composition that is capable of inducing an immune response to the at least one neoantigen, and
wherein the subject was previously administered a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the second and third compositions are administered concurrently or sequentially to the subject, wherein the first peptide composition and the second peptide composition are each capable of inducing an immune response to the at least one neoantigen, and wherein the first oncolytic virus is immunologically distinct from the second oncolytic virus.

64. The method of claim 62 or 63, wherein the immune response to the at least one neoantigen that is induced in the subject comprises a peak immune response to the at least one neoantigen with the second boost that is at least 0.5 log higher than the peak immune response to the at least one neoantigen attained with the first boost.

65. The method of claim 62, 63 or 64, wherein one month after the second boost the immune response to the at least one neoantigen remains higher that the peak immune response to the at least one neoantigen attained with the first boost.

66. The method of claim 64 or 65, wherein the immune response is measured by the number of antigen-specific interferon gamma-positive CD8+ T cells per ml of peripheral blood from the subject.

67. The method of any one of claims 62 to 66, wherein the first oncolytic virus comprises a genome that comprises a first transgene, wherein the first transgene encodes and expresses a first protein in the subject, and wherein the first protein or a fragment thereof is capable of inducing an immune response to the at least one neoantigen.

68. The method of any one of claims 62 to 66, wherein the second oncolytic virus comprises a genome that comprises a second transgene, wherein the second transgene encodes and expresses a second protein in the subject, and wherein the second protein or a fragment thereof is capable of inducing an immune response to the at least one neoantigen.

69. The method of any one of claims 62 to 68, wherein the method further comprises administering to the subject a third boost comprising a dose of a seventh composition, wherein the seventh composition comprises a third oncolytic virus comprising a genome that comprises a third transgene, wherein the third transgene encodes and expresses a third protein in the subject, wherein the third protein or a fragment thereof is capable of inducing an immune response to the at least one neoantigen, and wherein the third oncolytic virus is immunologically distinct from the second oncolytic virus.

70. The method of any one of claims 62 to 68, wherein the method further comprises administering to the subject a third boost comprising:
(i) a dose of a seventh composition comprising a third oncolytic virus and a third peptide composition; or
(ii) a dose of an eighth composition and a dose of a ninth composition, wherein the eighth composition comprises the third oncolytic virus, and the ninth composition comprises the third peptide composition, wherein the eighth and ninth compositions are concurrently or sequentially administered to the subject, wherein the third peptide composition is capable of inducing an immune response to the at least one neoantigen, and wherein the third oncolytic virus is immunologically distinct from the second oncolytic virus.

71. The method of claim 69 or 70, wherein the third oncolytic virus is immunologically distinct from the first oncolytic virus.

72. The method of any one of claims 62 to 71, wherein the first composition is administered to the subject intravenously or intramuscularly.

73. The method of any one of claims 62 to 71, wherein the second composition is administered to the subject intravenously or intramuscularly.

74. The method of any one of claims 62 to 71 and 73, wherein the third composition is administered to the subject intravenously or intramuscularly.

75. The method of any one of claims 62 to 74, wherein the fourth composition is administered to the subject intravenously or intramuscularly.

76. The method of any one of claims 62 to 74, wherein the fifth composition is administered to the subject intravenously or intramuscularly.

77. The method of any one of claims 62 to 74 and 76, wherein the sixth composition is administered to the subject intravenously or intramuscularly.

78. The method of any one of claims 62 to 77, wherein the first peptide composition and the second peptide composition each comprise an identical peptide.

79. The method of any one of claims 62 to 77, wherein the first peptide composition and the second peptide composition each comprise a peptide, wherein the peptide of the first peptide composition comprises an amino acid sequence that overlaps with an amino acid sequence of the peptide of the second peptide composition.

80. The method of any one of claims 62 to 77, wherein the first peptide composition comprises two peptides and the second peptide composition comprises two peptides, wherein the two peptides of the first and second peptide compositions are identical.

81. The method of any one of claims 62 to 77, wherein the first peptide composition comprises two peptides and the second peptide composition comprises two peptides, wherein the two peptides of the first and second peptide compositions each comprise overlapping amino acid sequences.

82. The method of any one of claims 62 to 81, wherein the first oncolytic virus, the second oncolytic virus, or both are attenuated.

83. The method of any one of claims 62 to 82, wherein the first or second oncolytic virus is a rhabdovirus.

84. The method of any one of claims 62 to 82, wherein the first or second oncolytic virus is a Maraba virus, a Farmington virus, an adenovirus, a measles virus or a vesicular stomatitis virus.

85. The method of claim 84, wherein the Maraba virus is MG1.

86. The method of any one of claims 62 to 82, wherein the first oncolytic virus is a Farmington virus and the second oncolytic virus is a Maraba virus.

87. The method of any one of claims 62 to 82, wherein the first oncolytic virus is a Maraba virus and the second oncolytic virus is a Farmington virus.

88. The method of claim 86 or 87, wherein the Maraba virus is MG1.

89. The method of any one of claims 62 to 82, wherein the first or second oncolytic virus is a vaccinia virus.

90. The method of any one of claims 62 to 82, wherein the first oncolytic virus is a vaccinia virus and the second oncolytic virus is a Maraba virus.

91. The method of any one of claims 62 to 82, wherein the first oncolytic virus is a Maraba virus and the second oncolytic virus is a vaccinia virus.

92. The method of claim 90 or 91, wherein the Maraba virus is MG1.

93. The method of any one of claims 62 to 82, wherein the first oncolytic virus is a vaccinia virus and the second oncolytic virus is a Farmington virus.

94. The method of any one of claims 62 to 82, wherein the first oncolytic virus is a Farmington virus and the second oncolytic virus is a vaccinia virus.

95. The method of any one of claims 89 to 94, wherein the vaccinia virus is Copenhagen, Western Reserve, Wyeth, Tian Tan or Lister.

96. The method of any one of claims 62 to 95, wherein the subject has been determined to have pre-existing immunity to the at least one neoantigen.

97. The method of claim 96, wherein the subject is determined to have pre-existing immunity by measuring the number of antigen-specific interferon gamma-positive CD8+ T cells per ml of peripheral blood from the subject.

98. The method of any one of claims 62 to 95, wherein the subject was previously administered a dose of the priming composition.

99. The method of claim 98, wherein the subject was administered the dose of the priming composition 7 to 21 days before the subject was administered the first boost.

100. The method of claim 98, wherein the subject was administered the dose of the priming composition 2 weeks to 3 months before the subject was administered the first boost.

101. The method of claim 98, 99 or 100, wherein the priming composition comprises: (i) a nucleic acid sequence, wherein the nucleic acid sequence encodes and expresses a first priming protein in the subject, wherein the first priming protein or a fragment thereof is capable of inducing an immune response to the at least one neoantigen, (ii) a priming peptide composition, wherein the priming peptide composition is capable of inducing an immune response to the at least one neoantigen, (iii) an adoptive cell transfer of CD8+ T cells specific for the at least one neoantigen, (iv) a first priming virus that comprises a genome comprising a first priming transgene, wherein the first priming transgene encodes and expresses a second priming protein in the subject, wherein the second priming protein or a fragment thereof is capable of inducing an immune response to the at least one neoantigen, or (v) a second priming virus and a first priming peptide composition, and wherein the first priming virus and the second priming virus are immunologically distinct from the first oncolytic virus.

102. The method of claim 101, wherein the first priming virus and the second priming virus are immunologically distinct from the second oncolytic virus.

103. The method of claim 101 or 102, wherein the priming composition further comprises an adjuvant.

104. The method of claim 103, wherein the adjuvant is poly I:C.

105. The method of any one of claims 101 to 104, wherein the priming composition further comprises a liposome.

106. The method of any one of claims 62 to 105, wherein the third composition further comprises a liposome or a nanoparticle.

107. The method of any one of claims 62 to 106, wherein the sixth composition further comprises a liposome or a nanoparticle.

108. A method of inducing an immune response to at least one neoantigen in a subject, comprising:
(a) administering to the subject a dose of a priming composition that is capable of inducing an immune response to the at least one neoantigen;
(b) subsequently administering to the subject a first boost comprising (i) a dose of a first composition, wherein the first composition comprises a first oncolytic virus and a first peptide composition, or (ii) a dose of a second composition and a dose of third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and
(c) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the second peptide composition is capable of inducing an immune response to the at least one neoantigen, wherein the fifth and sixth compositions are administered concurrently or sequentially to the subject, and wherein second oncolytic virus is immunologically distinct from the first oncolytic virus.

109. The method of claim 108, wherein the first oncolytic virus comprises a genome that comprises a first transgene, wherein the first transgene encodes and expresses a first protein in the subject, and wherein the first protein or a fragment thereof is capable of inducing an immune response to the at least one neoantigen.

110. The method of claim 108 or 109, wherein the second oncolytic virus comprises a genome that comprises a second transgene, wherein the second transgene encodes and expresses a second protein in the subject, and wherein the second protein or a fragment thereof is capable of inducing an immune response to the at least one neoantigen.

111. The method of any one of claims 108 to 110, wherein the method further comprises administering to the subject a third boost comprising a dose of a seventh composition, wherein the seventh composition comprises a third oncolytic virus comprising a genome that comprises a third transgene, wherein the third transgene encodes and expresses a third protein in the subject, wherein the third protein or a fragment thereof is capable of inducing an immune response to the at least one neoantigen, and wherein the third oncolytic virus is immunologically distinct from the second oncolytic virus.

112. The method of any one of claims 108 to 110, wherein the method further comprises administering to the subject a third boost comprising:
(i) a dose of a seventh composition comprising a third oncolytic virus and a third peptide composition; or
(ii) a dose of an eighth composition and a dose of a ninth composition, wherein the eighth composition comprises the third oncolytic virus, and the ninth composition comprises the third peptide composition, wherein the eighth and ninth compositions are concurrently or sequentially administered to the subject, wherein the third peptide composition is capable of inducing an immune response to the at least one neoantigen, and wherein the third oncolytic virus is immunologically distinct from the second oncolytic virus.

113. The method of claim 110 or 112, wherein the third oncolytic virus is immunologically distinct from the first oncolytic virus.

114. The method of any one of claims 108 to 113, wherein the first composition is administered to the subject intravenously or intramuscularly.

115. The method of any one of claims 108 to 113, wherein the second composition is administered to the subject intravenously or intramuscularly.

116. The method of any one of claims 108 to 113 and 115, wherein the third composition is administered to the subject intravenously or intramuscularly.

117. The method of any one of claims 108 to 115, wherein the fourth composition is administered to the subject intravenously or intramuscularly.

118. The method of any one of claims 108 to 115, wherein the fifth composition is administered to the subject intravenously or intramuscularly.

119. The method of any one of claims 108 to 115 and 118, wherein the sixth composition is administered to the subject intravenously or intramuscularly.

120. The method of any one of claims 108 to 119, wherein the first peptide composition and the second peptide composition each comprise an identical peptide.

121. The method of any one of claims 108 to 119, wherein the first peptide composition and the second peptide composition each comprise a peptide, wherein the peptide of the first peptide composition comprises an amino acid sequence that overlaps with an amino acid sequence of the peptide of the second peptide composition.

122. The method of any one of claims 108 to 119, wherein the first peptide composition comprises two peptides and the second peptide composition comprises two peptides, wherein the two peptides of the first and second peptide compositions are identical.

123. The method of any one of claims 108 to 119, wherein the first peptide composition comprises two peptides and the second peptide composition comprises two peptides, wherein the two peptides of the first and second peptide compositions each comprise overlapping amino acid sequences.

124. The method of any one of claims 108 to 123, wherein the third composition further comprises an adjuvant.

125. The method of any one of claims 108 to 124, wherein the sixth composition further comprises an adjuvant.

126. The method of any one of claims 108 to 125, wherein the third composition further comprises a liposome or a nanoparticle.

127. The method of any one of claims 108 to 126, wherein the sixth composition further comprises a liposome or a nanoparticle.

128. The method of any one of claims 108 to 127, wherein the priming composition is administered to the subject 7 to 21 days before the first boost.

129. The method of any one of claims 108 to 127, wherein the priming composition is administered to the subject two weeks to 3 months before the first boost.

130. A method of inducing an immune response to at least one neoantigen in a subject with pre-existing immunity to the at least one neoantigen, or a subject who has previously been administered a dose of a priming composition that is capable of inducing an immune response to the at least one neoantigen, the method comprising:
(a) administering to the subject a first boost comprising a dose of a first composition, wherein the first composition comprises a first oncolytic virus that comprises a genome comprising a first transgene, wherein the first transgene encodes and expresses a first protein in the subject, and wherein the first protein or a fragment thereof is capable of inducing an immune response to the at least one neoantigen; and
(b) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

131. A method of inducing an immune response to at least one neoantigen in a subject, the method comprising to the subject a second boost comprising a dose of a second composition, wherein the second composition comprises a second oncolytic virus that comprises a genome comprising a second transgene, wherein the second transgene encodes and expresses a second protein in the subject, wherein the second peptide or a fragment thereof is capable of inducing an immune response to the at least one neoantigen,
wherein the subject has pre-existing immunity to the at least one neoantigen, or the subject was previously administered a dose of a priming composition that is capable of inducing an immune response to the at least one neoantigen, and
wherein the subject was previously administered a first boost comprising a dose of a first composition, wherein the first composition comprises a first oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

132. A method of inducing an immune response to at least one neoantigen in a subject, comprising:
(a) administering to the subject a dose of a priming composition that is capable of inducing an immune response to the at least one neoantigen; and
(b) subsequently administering to the subject a first boost comprising a dose of a first composition, wherein the first composition comprises a first oncolytic virus that comprises a genome comprising a first transgene, wherein the first transgene encodes and expresses a first protein in the subject, and wherein the first protein or a fragment thereof is capable of inducing an immune response to the at least one neoantigen; and
(c) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

133. The method of claim 131, wherein the first boost was administered to the subject 7 to 21 days before the second boost.

134. The method of claim 131, wherein the first boost was administered to the subject two weeks to 3 months before the second boost.

135. The method of claim 130 or 131, wherein the priming composition was administered to the subject 7 to 21 days before the first boost.

136. The method of claim 130 or 131, wherein the priming composition was administered to the subject two weeks to 3 months before the first boost.

137. The method of claim 132, wherein the second boost is administered to the subject 7 to 21 days after the first boost.

138. The method of claim 132, wherein the second boost is administered to the subject two weeks to 3 months after the first boost.

139. The method of claim 132, 137 or 138, wherein the priming composition was administered to the subject 7 to 21 days before the first boost.

140. The method of claim 132, 137 or 138, wherein the priming composition was administered to the subject two weeks to 3 months before the first boost.

141. The method of claim 130, 131, 133 or 134, wherein the subject has pre-existing immunity to the at least one neoantigen.

142. The method of claim 141, wherein the subject is determined to have pre-existing immunity by measuring the number of antigen-specific interferon gamma-positive CD8+ T cells per ml of peripheral blood from the subject.

143. The method of any one of claims 130 to 142, wherein the first protein or fragment thereof and the second protein or fragment thereof are each capable of inducing an immune response to two or more different neoantigens.

144. The method of any one of claims 130 to 142, wherein the first protein comprises at least one epitope of each of the two or more neoantigens, and the second protein comprises at least one epitope of each of the two or more neoantigens.

145. The method of any one of claims 130 to 142, wherein the first protein encoded by the first transgene, the second protein encoded by the second transgene, or both include at least one proteasomal cleavage site.

146. The method of any one of claims 130 to 145, wherein the first protein encoded by the first transgene, the second protein encode by the second transgene, or both are a fusion protein.

147. The method of any one of claims 130 to 146, wherein the first composition is administered to the subject intravenously or intramuscularly.

148. The method of any one of claims 130 to 147, wherein the second composition is administered to the subject intravenously or intramuscularly.

149. The method of any one of claims 130 to 148, wherein the method further comprises administering the subject a third boost comprising (i) a dose of third composition comprising a third oncolytic virus that comprises a genome comprising a third transgene wherein the third transgene encodes and expresses a third protein in the subject, wherein the third protein or a fragment thereof is capable of inducing an immune response to the at least one neoantigen, (ii) a dose of fourth composition comprising a fourth oncolytic virus and a first peptide composition, wherein the first peptide composition is capable of inducing an immune response to the at least one neoantigen, or (iii) a dose of a fifth composition and a dose of a sixth composition, wherein the fifth composition comprises the fourth oncolytic virus, and the sixth composition comprises the first peptide composition, and wherein the third oncolytic virus and the fourth oncolytic virus are immunologically distinct from the second oncolytic virus.

150. The method of claim 149, wherein the third oncolytic virus and the fourth oncolytic virus are immunologically distinct from the first oncolytic virus.

151. A method of inducing an immune response to at least one neoantigen in a subject, comprising:
(a) administering to the subject a dose of a priming composition that is capable of inducing an immune response to the at least one neoantigen;
(b) subsequently administering to the subject a first boost comprising (i) a dose of a first composition, wherein the first composition comprises a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject; and
(c) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

152. A method of inducing an immune response to at least one neoantigen in a subject, comprising:
(a) administering to the subject a dose of a priming composition that is capable of inducing an immune response to the at least one neoantigen; and
(b) subsequently administering to the subject a first boost comprising a dose of a first composition, wherein the first composition comprises a first oncolytic virus that comprises a genome comprising a first transgene, wherein the first transgene encodes and expresses a first protein in the subject, wherein the first protein or a fragment thereof is capable of inducing an immune response to the at least one neoantigen; and
(c) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, and wherein the third and fourth compositions are administered concurrently or sequentially to the subject, and wherein the second oncolytic virus is immunologically distinct from the first oncolytic virus.

153. The method of claim 151, wherein the first composition is administered to the subject intravenously or intramuscularly.

154. The method of claim 151, wherein the second composition is administered to the subject intravenously or intramuscularly.

155. The method of claim 151 or 154, wherein the third composition is administered to the subject intravenously or intramuscularly.

156. The method of any one of claims 151 and 153 to 155, wherein the fourth composition is administered to the subject intravenously or intramuscularly.

157. The method of any one of claims 151 and 153 to 156, wherein the third composition further comprises an adjuvant.

158. The method of any one of claims 151 and 153 to 157, wherein the third composition further comprises a liposome or a nanoparticle.

159. The method of claim 152, wherein the first composition is administered to the subject intravenously or intramuscularly.

160. The method of claim 152 or 159, wherein the second composition is administered to the subject intravenously or intramuscularly.

161. The method of claim 152 or 159, wherein the third composition is administered to the subject intravenously or intramuscularly.

162. The method of any one of claims 152, 159 and 161, wherein the fourth composition is administered to the subject intravenously or intramuscularly.

163. The method of any one of claims 152 and 159 to 162, wherein the fourth composition further comprises an adjuvant.

164. The method of any one of claims 152 and 159 to 163, wherein the fourth composition further comprises a liposome or a nanoparticle.

165. The method of any one of claims 151 and 153 to 158, wherein the first oncolytic virus comprises a genome that comprises a second transgene, wherein the second transgene encodes and expresses a second protein in the subject, wherein the second protein or a fragment thereof is capable of inducing an immune response to the at least one neoantigen.

166. The method of any one of claims 152 and 159 to 164, wherein the second oncolytic virus comprises a genome that comprises a second transgene, wherein the second transgene encodes and expresses a second protein in the subject, wherein the second protein or a fragment thereof is capable of inducing an immune response to the at least one neoantigen.

167. The method of any one of claims 151 to 166, wherein the first protein or fragment thereof is capable of inducing an immune response to two or more different neoantigens.

168. The method of claim 167, wherein the first protein comprises at least one epitope of each of the two or more neoantigens.

169. The method of any one of claims 151 to 168, wherein the first protein encoded by the first transgene includes at least one proteasomal cleavage site.

170. The method of any one of claims 151 to 169, wherein the first protein encoded by the first transgene is a fusion protein.

171. The method of any one of claims 151 to 170, wherein the first peptide composition is capable of inducing an immune response to two or more different neoantigens.

172. The method of claim 171, wherein the first peptide composition comprises two peptides, wherein one of the peptides comprises at least one epitope of one of the neoantigens, and the other peptide comprises at least one epitope of the other neoantigen.

173. The method of any one of claims 151 to 172, wherein the method further comprises administering a third boost comprising (i) a dose of fifth composition comprising a third oncolytic virus comprising a genome that comprises a third transgene, wherein the third transgene encodes and expresses a third protein in the subject, wherein the third protein or a fragment thereof is capable of inducing an immune response to the at least one neoantigen, or (ii) a dose of a sixth composition comprising a fourth oncolytic virus and a second peptide composition, or (iii) a dose of a seventh composition and a dose of an eighth composition, wherein the seventh composition comprises the fourth oncolytic virus, and the eighth composition comprises the second peptide composition, wherein the seventh and eighth compositions are administered concurrently or sequentially to the subject, wherein the second peptide composition is capable of inducing an immune response to the at least one neoantigen, and wherein the third oncolytic virus and the fourth oncolytic virus are immunologically distinct from second oncolytic virus.

174. The method of claim 173, wherein the third oncolytic virus and the fourth oncolytic virus are immunologically distinct from the first oncolytic virus.

175. The method of any one of claims 151 to 174, wherein the first boost is administered to the subject 7 to 21 days after the priming composition.

176. The method of any one of claims 151 to 174, wherein the first boost is administered to the subject 2 weeks to 3 months after the priming composition.

177. The method of any one of claims 151 to 176, wherein the second boost is administered to the subject 7 to 21 days after the first boost.

178. The method of any one of claims 151 to 176, wherein the second boost is administered to the subject 2 weeks to 3 months after the first boost.

179. The method of any one of claims 108 to 178, wherein the immune response to the at least one neoantigen that is induced in the subject comprises a peak immune response to the at least one neoantigen with the second boost that is at least 0.5 log higher than the peak immune response to the at least one neoantigen attained with the first boost.

180. The method of any one of claims 108 to 179, wherein one month after the second boost the immune response to the at least one neoantigen remains higher that the peak immune response to the at least one neoantigen attained with the first boost.

181. The method of claim 179 or 180, wherein the immune response is measured by the number of antigen-specific interferon gamma-positive CD8+ T cells per ml of peripheral blood from the subject.

182. The method of any one of claims 108 to 181, wherein the priming composition comprises: (i) a nucleic acid sequence, wherein the nucleic acid sequence encodes and expresses a first priming protein in the subject, wherein the first priming protein or a fragment thereof is capable of inducing an immune response to the at least one neoantigen, (ii) a priming peptide composition, wherein the priming peptide composition is capable of inducing an immune response to the at least one neoantigen, (iii) an adoptive cell transfer of CD8+ T cells specific for the at least one neoantigen, (iv) a first priming virus that comprises a genome comprising a first priming transgene, wherein the first priming transgene encodes and expresses a second priming protein in the subject, wherein the second priming protein or a fragment thereof is capable of inducing an immune response to the at least one neoantigen, or (v) a second priming virus and a first priming peptide composition, and wherein the first priming virus and the second priming virus are immunologically distinct from the first oncolytic virus.

183. The method of claim 182, wherein the first priming virus and the second priming virus are immunologically distinct from the second oncolytic virus.

184. The method of any one of claims 108 to 183, wherein the first oncolytic virus, the second oncolytic virus, or both are attenuated.

185. The method of any one of claims 108 to 184, wherein the first or second oncolytic virus is a rhabdovirus.

186. The method of any one of claims 108 to 184, wherein the first or second oncolytic virus is a Maraba virus, a Farmington virus, an adenovirus, a measles virus or a vesicular stomatitis virus.

187. The method of claim 186, wherein the Maraba virus is MG1.

188. The method of any one of claims 108 to 184, wherein the first oncolytic virus is a Farmington virus and the second oncolytic virus is a Maraba virus.

189. The method of any one of claims 108 to 184, wherein the first oncolytic virus is a Maraba virus and the second oncolytic virus is a Farmington virus.

190. The method of claim 188 or 189, wherein the Maraba virus is MG1.

191. The method of any one of claims 108 to 184, wherein the first or second oncolytic virus is a vaccinia virus.

192. The method of any one of claims 108 to 184, wherein the first oncolytic virus is a vaccinia virus and the second oncolytic virus is a Maraba virus.

193. The method of any one of claims 108 to 184, wherein the first oncolytic virus is a Maraba virus and the second oncolytic virus is a vaccinia virus.

194. The method of claim 192 or 193, wherein the Maraba virus is MG1.

195. The method of any one of claims 108 to 184, wherein the first oncolytic virus is a vaccinia virus and the second oncolytic virus is a Farmington virus.

196. The method of any one of claims 108 to 184, wherein the first oncolytic virus is a Farmington virus and the second oncolytic virus is a vaccinia virus.

197. The method of any one of claims 191 to 196, wherein the vaccinia virus is Copenhagen, Western Reserve, Wyeth, Tian Tan or Lister.

198. The method of any one of claims 1 to 197, wherein a dose of an oncolytic virus is 10⁷ to 10¹² PFU.

199. The method of any one of claims 1 to 198, wherein the subject is a mammal.

200. The method of any one of claims 1 to 198, wherein the subject is a human.
